SEQUENCE LISTING (107 sequences)

SEQ ID NO: 1 - skipped

SEQ ID NO: 2 - protein, 5 aa, synthetic construct
RAYAR

SEQ ID NO: 3 - protein, 5 aa, synthetic construct
KAYAK

SEQ ID NO: 4 - skipped

SEQ ID NO: 5 - protein, 7 aa, synthetic construct
  REPEAT 1: note = repeat 0 to 20 times
  VARIANT 1: note = independently any amino acid
  VARIANT 2: note = independently any positively charged amino acid
  VARIANT 3..5: note = independently any amino acid
  VARIANT 6: note = independently any positively charged amino acid
  REPEAT 7: note = repeat 0 to 20 times
  VARIANT 7: note = independently any amino acid
ZBXXXBZ

SEQ ID NO: 6 - protein, 29 aa, synthetic construct
QARILEADAEILRAYARILEAHAEILRAQ

SEQ ID NO: 7 - protein, 29 aa, synthetic construct
  REPEAT 7: note = 0 to 20 times
QAKILEADAEILKAYAKILEAHAEILKAQ

SEQ ID NO: 8 - protein, 23 aa, synthetic construct
ADAEILRAYARILEAHAEILRAQ

SEQ ID NO: 9 - protein, 29 aa, synthetic construct
  MOD_RES <1: note = N-terminal acetylation
  MOD_RES >29: note = C-terminal amidation
QARILEADAEILRAYARILEAHAEILRAQ

SEQ ID NO: 10 - protein, 29 aa, synthetic construct
  MOD_RES <1: note = N-terminal acetylation
  MOD_RES >29: note = C-terminal amidation
QAKILEADAEILKAYAKILEAHAEILKAQ

SEQ ID NO: 11 - protein, 23 aa, synthetic construct
  MOD_RES <1: note = N-terminal acetylation
  MOD_RES >23: note = C-terminal amidation
ADAEILRAYARILEAHAEILRAQ

SEQ ID NO: 12 - protein, 11 aa, synthetic construct
QQKFQFQFEQQ

SEQ ID NO: 13 - protein, 11 aa, synthetic construct
  MOD_RES <1: note = N-terminal acetylation
  MOD_RES >11: note = C-terminal amidation
QQKFQFQFEQQ

SEQ ID NO: 14 - protein, 8 aa, synthetic construct
FKFEFKFE

SEQ ID NO: 15 - protein, 7 aa, synthetic construct
KFQFQFE

SEQ ID NO: 16 - protein, 11 aa, synthetic construct
QQRFQFQFEQQ

SEQ ID NO: 17 - protein, 11 aa, synthetic construct
QQRFQWQFEQQ

SEQ ID NO: 18 - protein, 16 aa, synthetic construct
FEFEFKFKFEFEFKFK

SEQ ID NO: 19 - protein, 11 aa, synthetic construct
QQRFEWEFEQQ

SEQ ID NO: 20 - protein, 11 aa, synthetic construct
  SITE 3: note = ornithine
  SITE 5: note = ornithine
  SITE 7: note = ornithine
QQXFXWXFQQQ

SEQ ID NO: 21 - protein, 12 aa, synthetic construct
FKFEFKFEFKFE

SEQ ID NO: 22 - protein, 12 aa, synthetic construct
FKFQFKFQFKFQ

SEQ ID NO: 23 - protein, 16 aa, synthetic construct
AEAKAEAKAEAKAEAK

SEQ ID NO: 24 - protein, 16 aa, synthetic construct
AEAEAKAKAEAEAKAK

SEQ ID NO: 25 - protein, 16 aa, synthetic construct
AEAEAEAEAKAKAKAK

SEQ ID NO: 26 - protein, 16 aa, synthetic construct
RADARADARADARADA

SEQ ID NO: 27 - protein, 16 aa, synthetic construct
RARADADARARADADA

SEQ ID NO: 28 - protein, 33 aa, synthetic construct
SGRGYBLGGQGAGAAAAAGGAGQGGYGGLGSQG

SEQ ID NO: 29 - protein, 10 aa, synthetic construct
  VARIANT 4: note = Val, Ala, Ser, or Pro
  VARIANT 6: note = Val, Ala, Ser, or Pro
  VARIANT 8: note = Val, Ala, Ser, or Pro
  VARIANT 10: note = Val, Ala, Ser, or Pro
EWEXEXEXEX

SEQ ID NO: 30 - protein, 10 aa, synthetic construct
  VARIANT 3: note = Val, Ala, Ser, or Pro
  VARIANT 5: note = Val, Ala, Ser, or Pro
  VARIANT 7: note = Val, Ala, Ser, or Pro
  VARIANT 9: note = Val, Ala, Ser, or Pro
WKXKXKXKXK

SEQ ID NO: 31 - protein, 15 aa, synthetic construct
KWKVKVKVKVKVKVK

SEQ ID NO: 32 - protein, 16 aa, synthetic construct
LLLLKKKKKKKKLLLL

SEQ ID NO: 33 - protein, 21 aa, synthetic construct
VKVKVKVKVDPPTKVKVKVKV

SEQ ID NO: 34 - protein, 21 aa, synthetic construct
VKVKVKVKVDPPTKVKTKVKV

SEQ ID NO: 35 - protein, 21 aa, synthetic construct
KVKVKVKVKDPPSVKVKVKVK

SEQ ID NO: 36 - protein, 21 aa, synthetic construct
VKVKVKVKVDPPSKVKVKVKV

SEQ ID NO: 37 - protein, 21 aa, synthetic construct
VKVKVKTKVDPPTKVKTKVKV

SEQ ID NO: 38 - protein, 11 aa, synthetic construct
  VARIANT 5: note = Glu, Asp, or Asn
QQKFXFQFEQQ

SEQ ID NO: 39 - protein, 11 aa, synthetic construct
  VARIANT 6: note = Trp or Tyr
QQKFQXQFEQQ

SEQ ID NO: 40 - protein, 11 aa, synthetic construct
  VARIANT 7: note = Glu, Asp, or Asn
QQKFQFXFEQQ

SEQ ID NO: 41 - protein, 9 aa, synthetic construct
VEVKVEVKV

SEQ ID NO: 42 - protein, 12 aa, synthetic construct
VEVKVEVKVEVK

SEQ ID NO: 43 - protein, 9 aa, synthetic construct
VVVAAAEEE

SEQ ID NO: 44 - protein, 20 aa, synthetic construct
VEVEVEVEVEVEVEVEVEVE

SEQ ID NO: 45 - protein, 9 aa, synthetic construct
CGNKRTRGC

SEQ ID NO: 46 - protein, 21 aa, synthetic construct
VKVKVKVKVDPPTKVEVKVKV

SEQ ID NO: 47 - protein, 8 aa, synthetic construct
LRKKLGKA

SEQ ID NO: 48 - protein, 8 aa, synthetic construct
VVVVVVKK

SEQ ID NO: 49 - protein, 16 aa, synthetic construct
AEAKAEAKAEAKAEAK

SEQ ID NO: 50 - protein, 8 aa, synthetic construct
AEAKAEAK

SEQ ID NO: 51 - protein, 12 aa, synthetic construct
AEAEAEAEAKAK

SEQ ID NO: 52 - protein, 8 aa, synthetic construct
AEAEAKAK

SEQ ID NO: 53 - protein, 16 aa, synthetic construct
AEAEAKAKAEAEAKAK

SEQ ID NO: 54 - protein, 16 aa, synthetic construct
RADARADARADARADA

SEQ ID NO: 55 - protein, 16 aa, synthetic construct
RADARGDARADARGDA

SEQ ID NO: 56 - protein, 8 aa, synthetic construct
RADARADA

SEQ ID NO: 57 - protein, 16 aa, synthetic construct
RARADADARARADADA

SEQ ID NO: 58 - protein, 8 aa, synthetic construct
RARADADA

SEQ ID NO: 59 - protein, 16 aa, synthetic construct
RARARARADADADADA

SEQ ID NO: 60 - protein, 16 aa, synthetic construct
ADADADADARARARAR

SEQ ID NO: 61 - protein, 16 aa, synthetic construct
DADADADARARARARA

SEQ ID NO: 62 - protein, 16 aa, synthetic construct
RAEARAEARAEARAEA

SEQ ID NO: 63 - protein, 8 aa, synthetic construct
RAEARAEA

SEQ ID NO: 64 - protein, 16 aa, synthetic construct
KAKAKAKAEAEAEAEA

SEQ ID NO: 65 - protein, 16 aa, synthetic construct
AEAEAEAEAKAKAKAK

SEQ ID NO: 66 - protein, 16 aa, synthetic construct
KADAKADAKADAKADA

SEQ ID NO: 67 - protein, 8 aa, synthetic construct
KADAKADA

SEQ ID NO: 68 - protein, 17 aa, synthetic construct
AEAEAHAHAEAEAHAHA

SEQ ID NO: 69 - protein, 9 aa, synthetic construct
AEAEAHAHA

SEQ ID NO: 70 - protein, 16 aa, synthetic construct
HEHEHKHKHEHEHKHK

SEQ ID NO: 71 - protein, 8 aa, synthetic construct
HEHEHKHK

SEQ ID NO: 72 - protein, 16 aa, synthetic construct
FEFEFKFKFEFEFKFK

SEQ ID NO: 73 - protein, 8 aa, synthetic construct
FEFKFEFK

SEQ ID NO: 74 - protein, 16 aa, synthetic construct
LELELKLKLELELKLK

SEQ ID NO: 75 - protein, 8 aa, synthetic construct
LELELKLK

SEQ ID NO: 76 - protein, 12 aa, synthetic construct
KFDLKKDLKLDL

SEQ ID NO: 77 - protein, 8 aa, synthetic construct
FKFEFKFF

SEQ ID NO: 78 - protein, 8 aa, synthetic construct
FEFEFKFK

SEQ ID NO: 79 - protein, 20 aa, synthetic construct
RFRFRFRFRFRFRFRFRFRF

SEQ ID NO: 80 - protein, 30 aa, synthetic construct
YLLYSKGNGCPKDITSGGCYLIGNKDLDPE

SEQ ID NO: 81 - protein, 29 aa, synthetic construct
VDDIDYTQQQKIAAGKAISADAIPGGSVD

SEQ ID NO: 82 - protein, 32 aa, synthetic construct
GIAKAFRAPSIREVSPGFGTLTQGGASIMYGN

SEQ ID NO: 83 - protein, 4 aa, synthetic construct
SGSG

SEQ ID NO: 84 - protein, 4 aa, synthetic construct
SGSG

SEQ ID NO: 85 - protein, 8 aa, synthetic construct
CCCCSGSG

SEQ ID NO: 86 - protein, 10 aa, synthetic construct
GGGGGGGGGG

SEQ ID NO: 87 - protein, 4 aa, synthetic construct
GSGS

SEQ ID NO: 88 - protein, 4 aa, synthetic construct
SSSS

SEQ ID NO: 89 - protein, 4 aa, synthetic construct
GGGS

SEQ ID NO: 90 - skipped

SEQ ID NO: 91 - protein, 24 aa, synthetic construct
GGCGGCGGCGGCGGCGGCGGCGGC

SEQ ID NO: 92 - protein, 15 aa, synthetic construct
GGGGSGGGGSGGGGS

SEQ ID NO: 93 - protein, 5 aa, synthetic construct
KSGSG

SEQ ID NO: 94 - protein, 6 aa, synthetic construct
KKSGSG

SEQ ID NO: 95 - protein, 10 aa, synthetic construct
EAAAKEAAAK

SEQ ID NO: 96 - protein, 5 aa, synthetic construct
GGAAY

SEQ ID NO: 97 - protein, 20 aa, synthetic construct
ASPAAPAPASPAAPAPSAPA

SEQ ID NO: 98 - protein, 20 aa, synthetic construct
  MOD_RES >20: note = C-terminal amidation
ASPAAPAPASPAAPAPSAPA

SEQ ID NO: 99 - protein, 12 aa, synthetic construct
  SITE 3: note = cyclohexylalanine
  SITE 1: note = D-alanine
  SITE 12: note = D-alanine
AKXVAAWTLKAA

SEQ ID NO: 100 - protein, 15 aa, synthetic construct
QLVFNSISARALKAY

SEQ ID NO: 101 - protein, 49 aa, synthetic construct
  MOD_RES >49: note = C-terminal amidation
SGSGQQKFQFQFEQQSGSGYLLYSKGNGCPKDITSGGCYLIGNKDLDPE

SEQ ID NO: 102 - protein, 48 aa, synthetic construct
  MOD_RES >48: note = C-terminal amidation
SGSGQQKFQFQFEQQSGSGVDDIDYTQQQKIAAGKAISADAIPGGSVD

SEQ ID NO: 103 - protein, 51 aa, synthetic construct
  MOD_RES >51: note = C-terminal amidation
SGSGQQKFQFQFEQQSGSGGIAKAFRAPSIREVSPGFGTLTQGGASIMYGN

SEQ ID NO: 104 - protein, 27 aa, synthetic construct
  MOD_RES >27: note = C-terminal amidation
  SITE 3: note = cyclohexylalanine
  SITE 1: note = D-alanine
  SITE 12: note = D-alanine
AKXVAAWTLKAASGSGQQKFQFQFEQQ

SEQ ID NO: 105 - protein, 30 aa, synthetic construct
  MOD_RES >30: note = C-terminal amidation
QLVFNSISARALKAYSGSGQQKFQFQFEQQ

SEQ ID NO: 106 - protein, 27 aa, synthetic construct
  SITE 3: note = cyclohexylalanine
  SITE 1: note = D-alanine
  SITE 12: note = D-alanine
AKXVAAWTLKAASGSGQQKFQFQFEQQ

SEQ ID NO: 107 - protein, 30 aa, synthetic construct; identical to SEQ ID NO: 105

(19) United States
(12) Patent Application Publication
(10) Pub. No.: US 2025/0381256 A1
(43) Pub. Date: Dec. 18, 2025

(54) SUBLINGUAL NANOFIBER VACCINES AND METHODS OF MAKING AND USING SAME

(71) Applicant: Duke University, Durham, NC (US)

(72) Inventors: Joel Collier, Durham, NC (US); Sean Kelly, Durham, NC (US)

(21) Appl. No.: 18/881,172

(22) PCT Filed: Jul. 5, 2023
(86) PCT No.: PCT/US2023/069660

§ 371 Date: Jan. 3, 2025

Related U.S. Application Data
(60) Provisional application No. 63/358,373, filed on Jul. 5, 2022.

(51) Int. Cl. A61K 39/108; A61K 39/00; A61P 13/02; A61P 31/04

(52) U.S. Cl. CPC A61K 39/0258; A61P 13/02; A61P 31/04; A61K 2039/541; A61K 2039/55511; A61K 2039/55544; A61K 2039/55561

(57) ABSTRACT

Embodiments are directed to a UPEC conjugate peptide including a self-assembling peptide and at least one UPEC epitope. The UPEC conjugate peptide may self-assemble into a nanofiber or fibril. Compositions including the UPEC conjugate peptide may be used to treat a bacterial infection such as a urinary tract infection (UTI). The compositions and methods may be specific for pathogenic bacteria. The compositions and methods may treat the infection without altering the gut microbiome.

### CROSS-REFERENCE TO RELATED APPLICATIONS

This application claims priority to U.S. Provisional Patent Application No. 63/358,373, filed Jul. 5, 2022, which is incorporated herein by reference in its entirety.

### STATEMENT REGARDING FEDERALLY SPONSORED RESEARCH

This invention was made with government support under grants R01EB009701 and R01Al167300 awarded by the National Institutes of Health (NIH). This invention was made with government support under grant DGE-1644868 awarded by the National Science Foundation (NSF). The government has certain rights in the invention.

### FIELD

Embodiments of this invention are directed generally to biology, medicine, and immunology. Certain aspects are directed to peptide conjugates and nanofibers and their use in inducing an immune response to treat bacterial infections.

### INTRODUCTION

More than half of all women experience a urinary tract infection (UTI) in their lifetime, and UTIs are a persistent complication of indwelling urinary catheters. Those suffering with recurrent UTIs (more than three infections in a one-year period) experience considerable loss in their quality of life and are burdened with increased health-care costs. Recurrent UTIs are managed by long-term antibiotic prophylaxis, although this is not recommended until other behavioral or non-antibiotic options have been attempted. In addition to drug-specific adverse effects, the prolonged use of antibiotics alters the patient's microbiota. Antibiotic treatment alters microbes' metabolic activity, gene expression, and protein synthesis, in addition to reducing the diversity of the microbiota as a whole and favoring resistant populations.

The influence of antibiotics on the microbiome is reflected in the alarming rise of antibacterial resistance. Modelling has forecast that by 2050, current practices would result in 10 million additional deaths per year by infection and a global economic cost of $100 trillion. Antibiotic resistance compounds the effects already associated with prolonged antibiotic use, making it likely that safe, effective treatment and prevention of UTIs will become increasingly challenging. Uropathogenic Escherichia coli (UPEC), which cause about 80% of uncomplicated UTIs, have become increasingly resistant to commonly used antibiotics such as ampicillin, ciprofloxacin, and trimethoprim-sulfamethoxazole (TMP-SMX). The current prevalence of recurrent UTIs, combined with the increasing loss of antibiotic efficacy, suggest that a new form of UTI prevention is a significant and urgent unmet need.

A vaccine that raises protective, long-term antibody responses against UTI-causing bacteria has the potential to meet this need. However, such a vaccine does not currently exist, and there are significant challenges to its development. Currently, immune-modulating therapies are being explored for the treatment and prevention of recurrent UTIs, including orally delivered bacterial lysates of UPEC strains such as the commercially available OM-89 or sublingually delivered inactivated bacterial strains such as Uromune, which has reported results in humans. However, these approaches require extended dosing regimens (typically at least 3 months of daily dosing) and have not been shown to elicit long-lasting protection. The phase 1b trial of EXPEC4V, an intramuscular vaccine targeting the lipopolysaccharide-linked O-antigen of extraintestinal E. coli, showed no significant differences in UTI incidence. As evidenced by these clinical results, generating safe, effective, and long-lasting immune responses across populations for UTI prevention is a major challenge.

### SUMMARY

In an aspect, provided herein is a UPEC conjugate peptide. The UPEC conjugate peptide may include (i) a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and (ii) at least one UPEC epitope conjugated to a terminus of the self-assembling peptide, wherein the UPEC epitope is selected from plroN (YLLYSKGNGCPKDITSGGCYLIGNKDLDPE, SEQ ID NO: 80), plutA (VDDIDYTQQQKIAAGKAISADAIPGGSVD, SEQ ID NO: 81), or plreA (GIAKAFRAPSIREVSPGFGTLTQGGASIMYGN, SEQ ID NO: 82), or a combination thereof. In some embodiments, each self-assembling peptide forms a beta sheet and comprises a polypeptide having an amino acid sequence selected from QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn). In some embodiments, each self-assembling peptide forms an alpha-helix and comprises a polypeptide having an amino acid sequence of bXXXb (SEQ ID NO: 1), wherein X is independently any amino acid and b is independently any positively charged amino acid. In some embodiments, the self-assembling peptide comprises the sequence QQKFQFQFEQQ (SEQ ID NO: 12) or Ac-QQKFQFQFEQQ-NH₂ (SEQ ID NO: 13) or bXXXb (SEQ ID NO: 1, wherein X is independently any amino acid and b is independently any positively charged amino acid). In some embodiments, b is independently selected from Arg and Lys. In some embodiments, bXXXb (SEQ ID NO: 1) is RAYAR (SEQ ID NO: 2) or KAYAK (SEQ ID NO: 3). In some embodiments, the self-assembling peptide comprises an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20. In some embodiments, the self-assembling peptide comprises an amino acid sequence selected from QARILEADAEILRAYARILEAHAEILRAQ (Coil29, SEQ ID NO: 6), or QAKILEADAEILKAYAKILEAHAEILKAQ (SEQ ID NO: 7), or ADAEILRAYARILEAHAEILRAQ (SEQ ID NO: 8), or Ac-QARILEADAEILRAYARILEAHAEILRAQ-NH₂ (SEQ ID NO: 9), or Ac-QAKILEADAEILKAYAKILEAHAEILKAQ-NH₂ (SEQ ID NO: 10), or Ac-ADAEILRAYARILEAHAEILRAQ-NH₂ (SEQ ID NO: 11). In some embodiments, the at least one UPEC epitope is attached to the C-terminus or the N-terminus of the self-assembling peptide. In some embodiments, 1 to 10 UPEC epitopes are attached to the C-terminus or the N-terminus of the self-assembling peptide. In some embodiments, the UPEC conjugate peptide further includes (iii) a PEG molecule or a PAS peptide conjugated to the self-assembling peptide. In some embodiments, the PAS peptide comprises a sequence of Pro-Ala-Ser or comprises the sequence of ASPAAPAPASPAAPAPSAPA (SEQ ID NO: 97), or a peptide having at least 80%, 85%, 90%, or 95% identity thereto. In some embodiments, the PEG molecule comprises PEG-2000. In some embodiments, the PEG molecule or the PAS peptide is conjugated to the self-assembling peptide at the same or the opposite terminus from wherein the UPEC epitope is attached. In some embodiments, the UPEC conjugate peptide further includes (iv) at least one linker. In some embodiments, the at least one linker comprises a first linker between the at least one UPEC epitope and the self-assembling peptide, and a second linker between the PEG molecule or the PAS peptide and the self-assembling peptide. In some embodiments, the at least one linker comprises an amino acid sequence independently selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC)₈), SEQ ID NO: 92 ((G₄S)₃), SEQ ID NO: 93 (KSGSG), SEQ ID NO: 94 (KKSGSG), SEQ ID NO: 95 (EAAAK)₂, and SEQ ID NO: 96 (GGAAY). In some embodiments, the UPEC conjugate peptide comprises a sequence selected from PEG-Q11-plroN, PEG-Q11-plutA, or PEG-Q11-plreA, or a combination thereof.

In another aspect, provided herein is a nanofiber comprising a plurality of UPEC conjugate peptides as detailed herein, wherein the conjugate peptide self-assembles into the nanofiber. In another aspect, provided herein is a nanofiber including (i) at least one UPEC conjugate peptide as detailed herein; and (ii) at least one T-cell epitope-conjugate peptide. The at least one T-cell epitope-conjugate peptide may include a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and at least one T-cell epitope conjugated to a terminus of the self-assembling peptide, wherein the at least one T-cell epitope is selected from PADRE and VAC, and wherein PADRE comprises a polypeptide having the amino acid sequence of aKXVAAWTLKAa (SEQ ID NO: 99, wherein “X” comprises cyclohexylalanine and “a” comprises D-alanine), and wherein VAC comprises a polypeptide having the amino acid sequence of QLVFNSISARALKAY (SEQ ID NO: 100). In some embodiments, the T-cell epitope-conjugate peptide further comprises a linker between the T-cell epitope and the self-assembling peptide. In some embodiments, the linker comprises an amino acid sequence selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC)₈), SEQ ID NO: 92 ((G₄S)₃), SEQ ID NO: 93 (KSGSG), SEQ ID NO: 94 (KKSGSG), SEQ ID NO: 95 (EAAAK)₂, and SEQ ID NO: 96 (GGAAY). In some embodiments, the cyclohexylalanine comprises D-alanine. In some embodiments, the nanofiber further includes (iii) a plain self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn). In some embodiments, the nanofiber comprises a combination of 10 to 10,000, or 100 to 10,000 peptides comprising UPEC conjugate peptides and T-cell epitope-conjugate peptides. In some embodiments, the nanofiber comprises a combination of 10 to 10,000, or 100 to 10,000 peptides comprising UPEC conjugate peptides, T-cell epitope-conjugate peptides, and plain self-assembling peptides. In some embodiments, at least about 1%, 5%, 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, or 97.5% of the peptides in the nanofiber are UPEC conjugate peptides. In some embodiments, at least about 1%, 2%, 3%, 4%, 5%, 6%, 7%, 8%, 9%, 10%, 11%, 12%, 13%, 14%, or 15% of the peptides in the nanofiber are T-cell epitope-conjugate peptides. In some embodiments, the UPEC peptide conjugate and the T-cell epitope-peptide conjugate are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1. In some embodiments, the self-assembling peptide forms a fibril including beta-sheet structures or a fibril having a coiled coil structure. In some embodiments, the self-assembling peptide forms a fibril having a structure of a helical filament formed around a central axis. In some embodiments, the N-terminus of each self-assembling peptide is positioned at the exterior of the helical filament. In some embodiments, the UPEC molecules are exposed on the exterior surface of the nanofiber. In some embodiments, the nanofiber is about 5-30 nm in width. In some embodiments, the nanofiber is about 100 nm to 1 μm, 100 nm to 2 μm, 100 nm to 3 μm, 100 nm to 4 μm, or 100 nm to 5 μm in length.

In another aspect, provided herein is a pharmaceutical composition including (a) a UPEC conjugate peptide as detailed herein or a nanofiber as detailed herein; and (b) a pharmaceutically acceptable carrier, diluent, and/or excipient. In some embodiments, the pharmaceutical composition further includes (c) an adjuvant selected from cyclic-di-AMP, CpG, cyclic GMP-AMP (cGAMP), cholera toxin B subunit (CTB), retinoic acid, or heat labile toxin B subunit, or a combination thereof. In some embodiments, the pharmaceutical composition is formulated into a tablet. In some embodiments, the tablet is a dissolving tablet.

In another aspect, provided herein is a method of treating a urinary tract infection (UTI). The method may include administering to a subject a therapeutically effective amount of a UPEC conjugate peptide as detailed herein, or a nanofiber as detailed herein, or a pharmaceutical composition as detailed herein. In another aspect, provided herein is a method of treating a bacterial infection. The method may include administering to a subject a therapeutically effective amount of a UPEC conjugate peptide as detailed herein, or a nanofiber as detailed herein, or a pharmaceutical composition as detailed herein. In some embodiments, the UPEC conjugate peptide or the nanofiber or the pharmaceutical composition is administered sublingually. In some embodiments, pathogenic bacteria are reduced. In some embodiments, pathogenic bacteria comprise uropathogenic Escherichia coli. In some embodiments, pathogenic bacteria comprise CFT073. In some embodiments, non-pathogenic bacteria are not reduced. In some embodiments, the microbiome in the colon is maintained. In some embodiments, the microbiome in the colon statistically maintains a Shannon Diversity Index.

The disclosure provides for other aspects and embodiments that will be apparent in light of the following detailed description and accompanying figures.

### BRIEF DESCRIPTION OF THE DRAWINGS

FIGS. 1A-1G. Sublingual nanofiber vaccine raises antibody responses against three B-cell epitopes with broad expression across uropathogenic E. coli strains. Transmission electron microscopy images of nanofibers composed of (FIG. 1A) PEG-Q11plreA, (FIG. 1B) PEG-Q11plutA, or (FIG. 1C) PEG-Q11plroN. (FIG. 1D) Percentage of clinical UPEC isolates that contain the gene encoding the parent proteins of the plroN, plutA, and plreA epitopes (Hagan, E. C. Iron Acquisition by Uropathogenic Escherichia coli: ChuA and Hma Heme Receptors as Virulence Determinants and Vaccine Targets, 2009, incorporated herein by reference). (FIG. 1E) Mice were immunized sublingually with co-assembled PEG-Q11 nanofibers containing the PADRE T-helper epitope and either plreA, plroN, or plutA B-cell epitope, plus cholera toxin B (CTB) adjuvant. Mice were boosted at weeks 1 and 3, and serum IgG titer against the immunizing epitope was measured. **p<0.01 by 2-way RM-ANOVA with Tukey's multiple comparisons test, n=4/group. (FIG. 1F) Effects of titrating T-cell epitope content with sublingual nanofiber vaccines. Mice were immunized sublingually with PEG-Q11 (plreA/PADRE) nanofibers containing variable concentrations of PADRE, plus CTB, and were boosted at weeks 1, 3, and 6. n=4/group. (FIG. 1G) Co-assembly with the VAC epitope, but not PADRE, elicits antibody responses against plutA and plroN. Mice immunized with the plutA or plroN epitope and VAC were compared to responses with PADRE shown in FIG. 1F. All mice were boosted at weeks 1 and 3, and formulations contained CTB adjuvant. **p<0.01, *p<0.05 by 2-way RM-ANOVA with Tukey's multiple comparisons test, n=4/group.

FIGS. 2A-2C. T-cell responses with sublingual immunization against individual B-cell/T-cell epitope co-assemblies. ELISPOT was performed on splenocytes harvested from mice immunized in FIGS. 1A-1G. Mice were boosted with the immunizing formulation one week before sacrifice. Cells were stimulated with the immunizing T-cell epitope. SFC: spot-forming cells. Subscripts indicate the molar fraction of the T-cell epitope within the co-assembled nanofiber. (FIG. 2A-FIG. 2B) Corresponds to mice immunized with PEG-Q11 (plreA/PADRE) at differing doses of PADRE in FIG. 1F. Mice in FIG. 2A were sacrificed at week 73, and mice in FIG. 2B were sacrificed at week 64. (FIG. 2C) Corresponds to mice immunized with PEG-Q11 (plroN/VAC) or PEG-Q11 (plutA/VAC) in FIG. 1G, sacrificed at week 53.

FIGS. 3A-3H. Fully co-assembled nanofibers elicit polyvalent systemic and urinary antibody responses that specifically target uropathogenic E. coli. Mice were immunized sublingually with either a mixture of three separately assembled nanofibers (PEG-Q11 (plreA/PADRE)+PEG-Q11 (plutA/VAC)+PEG-Q11 (plroN/VAC)) or a single fully co-assembled nanofiber (PEG-Q11 (plreA/plutA/plroN)) and either cholera toxin B (CTB) or cyclic-di-AMP (c-di-AMP) adjuvant, and boosted at weeks 1, 3, 6, and 8. FIG. 3A is a schematic depicting the epitope composition of the single nanofiber and three nanofiber formulations. (FIG. 3B-FIG. 3D) IgG levels were measured individually against each of the three peptide epitopes. (FIG. 3E) To compare the overall response, a titer sum was calculated by arithmetic addition of the titers against each of the three epitopes. **p<0.01, * p<0.05 by 2-way RM-ANOVA with Tukey's multiple comparisons test, n=5/group. (FIG. 3F) Serum antibody isotype and IgG subclasses were measured by ELISA against a 1:1:1 mixture of plreA, plutA, and plroN. (FIG. 3G) Urinary antibody levels were determined by ELISA on undiluted urine samples. n.s.=non-significant by 2-way ANOVA, n=1-4/group. (FIG. 3H) Vaccine-induced serum antibodies bound specifically to uropathogenic E. coli. Week 13 serum IgG titers were measured by ELISA against a uropathogenic E. coli strain (CFT073) or a non-pathogenic lab strain (BL21). *p<0.05 by multiple t-tests with Holm-Šídák correction, n=4-5/group.

FIGS. 4A-4L. Formulation with STING and TLR9 agonists promotes strong serum and urinary antibodies against uropathogenic E. coli without accompanying gut responses. (FIG. 4A) Mice were immunized with PEG-Q11 (plreA/plutA/plroN/VAC) nanofibers, plus cyclic-di-AMP (c-di-AMP) adjuvant alone or in combination with CpG, CRX527, or C48/80 adjuvants. Mice were boosted at weeks 1, 3, 6, and 10. To compare the overall response, a titer sum was calculated by arithmetic addition of the titers against each of the three epitopes. Antibody titers of unimmunized mice were measured at week 36. n.s.=non-significant by 2-way RM-ANOVA, n=5/group. (FIG. 4B) Heat map showing the plreA-, plutA-, and plroN-specific serum antibody response for each group at week 36. (FIG. 4C) Week 11 antibody isotype and IgG subclasses were measured by diluting serum 1:1000 for ELISA against a 1:1:1 mixture of plreA, plutA, and plroN. (FIG. 4D) Week 11 serum IgG was measured by ELISA against a uropathogenic E. coli strain (CFT073) or a non-pathogenic lab strain (BL21). ***p<0.001, **p<0.01, *p<0.05 by multiple t-tests with Holm-Šídak correction. (FIG. 4E-FIG. 4F) Urinary IgA and IgG at week 11 was measured by ELISA on undiluted urine against a 1:1:1 mixture of plreA, plutA, and plroN. **p<0.01, n.s.=non-significant by 1-way ANOVA with Dunnett's multiple comparisons test against c-di-AMP only group. (FIG. 4G) No antigen-specific fecal antibodies were observed after sublingual immunizations. Fecal IgA was measured by ELISA against a 1:1:1 mixture of plreA, plutA, and plroN using fecal extract. (FIG. 4H) Experimental timeline. Mice immunized in FIG. 4A were challenged intraperitoneally 26 weeks after the final boost with a 1×10⁸ CFU of a uropathogenic E. coli strain (CFT073) cultured in Luria broth. These culture conditions induced expression of lutA, but not IreA or IroN (Hagan, E. C. Iron Acquisition by Uropathogenic Escherichia coli: ChuA and Hma Heme Receptors as Virulence Determinants and Vaccine Targets, 2009, incorporated herein by reference). Mice were monitored for 72 h post-challenge. (FIG. 4I) Lowest temperature recorded for each mouse during the duration of the study. *p<0.05 by 1-way ANOVA with Dunnett's multiple comparisons test against unimmunized group, n=4-5/group. (FIG. 4J) Body weight was measured over time. Percent of weight loss was calculated using the weight of mice just prior to challenge. Bold lines represent the mean for each group, and the shaded boundaries indicate SEM. Weight and temperature curves for individual mice are in FIGS. 6A-6E. (FIG. 4K) Immunized groups were combined to show the vaccine's amelioration of weight loss vs unimmunized mice. *p<0.05 by 2-way RM-ANOVA, n=4 (unimmunized) or n=17 (combined immunization groups). (FIG. 4L) Survival curve for unimmunized vs. combined immunization groups. Mice were sacrificed at humane endpoints. **p<0.01 by log-rank test.

FIG. 5. Total (non-antigen specific) IgA levels in serum, fecal extracts, and urine. Total IgA in sera, fecal extract, and urine were determined for mice immunized in FIG. 4A using an ELISA kit. n.s.=non-significant by 2-way ANOVA with Tukey's multiple comparisons test, n=5/group.

FIGS. 6A-6E. Body weight (left) and temperature (right) curves for individual mice in sepsis challenge from FIG. 4H. FIG. 6A is for unimmunized, FIG. 6B is for Tablet+c-di-AMP, FIG. 6C is for Tablet+c-di-AMP/CpG, FIG. 6D is for Tablet+c-di-AMP/CRX527, and FIG. 6E is for Tablet+c-di-AMP/C48/80.

FIGS. 7A-7N. A highly accessible tablet delivery vehicle enables sublingual immunization against uropathogenic E. coli epitopes in mice and rabbits. (FIG. 7A) Schematic depicting tablet-making process. (FIG. 7B) Camera image of tablet vaccine (tablet diameter is 5 mm). (FIG. 7C) Heat map showing the plreA-, plutA-, and plroN-specific serum antibody response for each group at week 14. Mice were immunized with tablets containing PEG-Q11 (plreA/plutA/plroN/VAC) or PEG-Q11 (plreA/plutA/plron; no VAC) and cyclic-di-AMP (c-di-AMP) adjuvant alone or both c-di-AMP and CpG adjuvants. (FIG. 7D) Tablets containing nanofibers with the VAC T-cell epitope elicited higher overall serum IgG responses than tablets lacking the T-cell epitope. Mice were boosted at weeks 1, 6, and 8 (arrows). **p<0.01 by 2-way RM-ANOVA with Dunnett's multiple comparisons test against No VAC tablet group, n=5/group. (FIG. 7E-FIG. 7F) Urinary IgA and IgG at week 9 was measured by ELISA in undiluted urine against a 1:1:1 mixture of plreA, plutA, and plroN. *p<0.05 by 1-way ANOVA with Dunnett's multiple comparisons test against No VAC group. n=4/group in E due to lack of sufficient urine for some mice to run undiluted ELISAs. (FIG. 7G) Experimental timeline for UPEC challenge experiment. (FIG. 7H) Mouse temperatures were recorded prior to infection and for the duration of the study. Bold lines represent the mean for each group, and the shaded boundaries indicate SEM. 2-way RM-ANOVA with Dunnett's multiple comparisons test against unimmunized group. N=5/group. (FIG. 7I) Survival curve. Mice were sacrificed at humane endpoints or at 72 hours. Weight and temperature curves for individual mice are in FIGS. 8A-8D. (FIG. 7J) Contingency table depicting outcomes for mice in immunized (n=5) or immunized (n=15) groups. *p<0.05 by Fisher's exact test. (FIG. 7K) Camera image showing placement of tablet vaccine under the tongue of an anesthetized rabbit. (FIG. 7L) Rabbits were immunized with vaccine tablets containing PEG-Q11 (PireA/PiutA/PiroN/VAC) or PEG-Q11 (PireA/lutA/IroN; no VAC), plus c-di-AMP and CpG adjuvants, and boosted at weeks 2, 4, 7, and 10. Serum antibody responses at week 11 were measured by ELISA. N=4/group (tablets containing VAC) or n=3/group (No VAC tablets). (FIG. 7M) Urinary antibody responses in rabbits were measured at week 11 by ELISA on undiluted urine against a 1:1:1 mixture of PireA, PiutA, and PiroN. *p<0.05 by two-way ANOVA with Šidák's multiple comparisons test. (FIG. 7N) Week 11 serum from rabbits was assayed by ELISA for binding against non-pathogenic BL21 E. coli, or the UPEC strain CFT073 cultured under normal or iron-limited conditions. *p<0.05 by two-way ANOVA with Tukey's multiple comparison test.

FIGS. 8A-8D. Body weight (left) and temperature (right) curves for individual mice in sepsis challenge in FIG. 7G. FIG. 8A is for Unimmunized, FIG. 8B is for No VAC Tablet+c-di-AMP, FIG. 8C is for Tablet+c-di-AMP, and FIG. 8D is for Tablet+c-di-AMP/CpG.

FIGS. 9A-9M. Sublingual anti-UPEC vaccine is as effective as high-dose antibiotics at preventing transurethral infection, but without accompanying disruption of the microbiome. (FIG. 9A) Experimental timeline. Mice (n=10/group) were immunized sublingually against UPEC with a vaccine containing PEG-Q11 (plreA/plroN/plutA) plus cyclic-di-AMP (c-di-AMP) and CpG adjuvants and boosted at weeks 2, 4, 9, and 12. Control groups were either left unimmunized or immunized against an irrelevant T- and B-cell epitope (OVA₃₂₃₋₃₃₉) using a PEG-Q11OVA vaccine containing the same Q11, epitope, and adjuvant concentrations as the anti-UPEC vaccine. Mice were infected transurethrally with 5×10⁷ CFU of CFT073 uropathogenic E. coli and sacrificed 48 h or at humane endpoints. Unimmunized mice were given oral antibiotics (Fosfomycin) at 1000 mg/kg daily for 3 days prior to infection and during the course of the infection (6 total doses). (FIG. 9B) Serum antibody responses in the anti-UPEC vaccine group were measured by ELISA over time against plreA, plroN, and plutA, and an arithmetic titer sum was calculated. Bold line represents the mean titer, and faded lines represent individual mice. Individual titer curves for antibodies against plreA, plron, and pluta are in FIGS. 11A-11C. (FIG. 9C) Serum IgG titers against the OVA₃₂₃₋₃₃₉ epitope in mice given the control vaccine against OVA. (FIG. 9D) Serum antibody responses against a 1:1:1 mixture or plreA, plutA, and plroN in unimmunized mice. (FIG. 9E) Survival curve. (FIG. 9F) Mouse temperatures were recorded prior to infection and for the duration of the study. 2-way RM-ANOVA with Dunnett's multiple comparisons test against the OVA control vaccine group at equivalent time points. Shaded areas depict SEM; bold line represents the mean. (FIG. 9G) Body weight was measured over time. Percent of weight loss was calculated using the weight of mice just prior to challenge. 2-way RM-ANOVA against the OVA control vaccine group. Shaded area depicts SEM; bold line represents the mean. Weight and temperature curves for individual mice are in FIGS. 12A-12C. (FIG. 9H-FIG. 9M) Fecal pellets were collected prior to treatment (week 0) and at week 13 for all groups, plus at week 13.5 from unimmunized groups (after 3 days of 1000 mg/kg daily treatment with Fosfomycin antibiotics) and used to compare the effects of vaccination and antibiotics on the microbiome. Richness sampling is shown in FIGS. 13A-13B. Estimation of the microbiome's OTU richness (FIG. 9H) and a-diversity (Shannon diversity index) (FIG. 9I). 1-way ANOVA with Tukey's multiple comparisons test. (FIG. 9J) Unweighted unifrac PCOA plot created using gut microbiome beta diversity from an OTU sample depth of 94,132. Each sample is plotted individually, and the relative closeness of points corresponds to their overall similarity. (FIG. 9K) Heatmap showing relative diversity of microbiome at the family taxonomic level. Each row represents an individual mouse at week 13 (post-vaccination) or week 13.5 (after antibiotic treatment). Full heatmap is shown in FIG. 14. (FIG. 9L-FIG. 9M) Over- and under-represented species in antibiotic-treated mice's microbiome at the species taxonomic level.

FIG. 10. Simulation of tablet disintegration in human oral cavity. Immunization formulation UTI tablets containing PEG-Q11 (plreA/plutA/plroN/VAC) nanofibers and cyclic-di-AMP adjuvant were placed into 1 mL of human saliva heated to 37° C., and disintegration time was measured. Solid line at 20 seconds represents the median disintegration time. n=5.

FIGS. 11A-11C. Individual epitope-specific antibody responses for mice from FIG. 9B. Antibody titers at week 13 titers were measured by ELISA against plreA (FIG. 11A), plutA (FIG. 11B), and plroN (FIG. 11C).

FIGS. 12A-12C. Body weight (left) and temperature (right) curves for individual mice in transurethral challenge in FIG. 9A. FIG. 12A is for Antibiotics, FIG. 12B is for Control Vaccine (OVA), and FIG. 12C is for Anti-UPEC Vaccine.

FIGS. 13A-13B. Richness sampling. Flattened curve in alpha rarefaction plot indicated the samples have been fully observed. FIG. 13A shows observed OTUs per sequencing depth, and FIG. 13B shows number of samples per sequencing depth.

FIG. 14. Complete family-level heatmap. Heatmap showing relative diversity of microbiome at the family taxonomic level. Each row represents an individual mouse.

FIG. 15. Coil29 nanofibers were sublingually immunogenic without PAS modification. Mice were immunized four times sublingually with nanofibers at week 0, 2, 4, and 10. Anti-OVA serum IgG measurements indicated a significant increase in titer for non-PASylated nanofibers (top, **p<0.01 by 2-way repeated measures ANOVA with Tukey's post hoc test, n=5). There was a higher but not statistically significant increase in vaginal IgA (bottom) for non-PASylated nanofibers.

FIG. 16. SIMPL tablets incorporating Coil29-based nanofibers. Top panel: epitope-containing peptide nanofibers were assembled in buffer, then cryoprotectant sugars were added, and tablets were formed by freeze drying. Bottom panel: Tablets were stable enough for handling (left), and Coil29 nanofibers retained their structure after freeze-drying, tabletization, and dissolution (images by atomic force microscopy).

FIG. 17. Serum IgG titers after sublingual immunization (arrows indicate immunization with SIMPL tablets). N=5 mice/group, immunized with OVA-Coil29 nanofibers at concentrations listed in a tablet made from 50 μL volume; all received 10 μg c-di-AMP.

FIG. 18. Serum IgG titers after sublingual immunization with Coil29 nanofibers with varying types and amounts of adjuvant. 1 μg c-di-AMP was sufficient to raise strong titers. Arrows indicate sublingual immunizations with liquid formulations of Coil29 nanofibers. N=5 mice/group, immunized with 8 μL of 5 mM OVA-Coil29.

### DETAILED DESCRIPTION

Provided herein are novel peptide conjugates that self-assemble into peptide nanofibers or fibrils, with an epitope for uropathogenic E. coli (UPEC) conjugated thereto. These peptide conjugates may be used to prevent and/or treat UTIs caused by uropathogenic E. coli. As detailed herein, the inventors employed a supramolecular approach to assemble multiple selected B-cell epitopes from uropathogenic E. coli (UPEC) into sublingually immunogenic nanomaterials. Sublingual immunization can elicit antibody responses in the urogenital tract, but it can be difficult to raise robust immune responses against short peptide epitopes via this route, because peptides are often poorly immunogenic via the oral mucosa. As demonstrated herein with model epitopes, supramolecular peptide nanofibers bearing polymer modifications modulating mucus adhesivity overcame these challenges and can raise strong systemic and mucosal antibody responses. This platform is based on multivalent peptide nanofibers, and antibody responses are raised and persist for at least a year. In some embodiments, the multivalent peptide nanofibers bear muco-inert modifications such as short polyethylene glycol (PEG) chains or Pro-Ala-Ser (PAS) peptides. The process of supramolecular assembly allows for the co-assembly of peptide-polymers bearing multiple selected pathogen-specific epitopes into integrated multi-epitope nanofibers.

In mice, this vaccine elicited robust anti-UPEC antibodies that were not cross-reactive against commensal E. coli. Further, the vaccines were as effective as high-dose oral antibiotics at protecting mice from lethal challenge with UPEC. Analysis of the composition of the gut microbiota demonstrated that the nanofiber vaccine caused significantly less perturbation than antibiotics. Both systemic responses in the blood and mucosal responses in the urogenital tract may be important for protection against UTIs. The compositions and methods detailed herein for UTI prevention can elicit immune responses that are specific to UTI-causing bacteria to avoid adverse effects to the microbiota, while also targeting a broad range of UTI-causing pathogens. Provided herein is a novel vaccination strategy, enabled by biomaterial design, that provides long-lasting, antibiotic-level efficacy against uropathogenic E. coli. The compositions and methods can raise simultaneous responses against multiple highly-specific and carefully selected epitopes targeting only pathogenic bacteria, an ability to elicit mucosal responses, and efficient dosing regimens that facilitate compliance and minimize cost.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

The term “adjuvant” refers to a compound or mixture that enhances the immune response to an antigen. Adjuvants may contain a substance to protect the antigen from rapid catabolism, such as aluminum hydroxide or a mineral oil, and also a protein derived from lipid A, Bortadella pertussis, or Mycobacterium tuberculosis. Suitable adjuvants may be commercially available and include, for example, complete or incomplete Freund's adjuvant; AS-2; aluminum salts such as aluminum hydroxide (as a gel, where appropriate) or aluminum phosphate; alum; MF59; calcium salts, iron salts, or zinc salts; an insoluble suspension of acylated tyrosine; acylated sugars; cationically or anionically derivatized polysaccharides; polyphosphazenes; biologically degradable microspheres; monophosphoryl lipid A, cytokines such as GM-CSF, Interleukin-2, Interleukin-7, Interleukin-12, CpG, cholera toxin B subunit (CTB), and/or STING agonists like cyclic dinucleotides.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

As used herein, the term “antigen” is a molecule capable of being bound by an antibody or T-cell receptor. The term “antigen”, as used herein, also encompasses T-cell epitopes. An antigen also refers to a molecule against which a subject can initiate a humoral and/or cellular immune response leading to the activation of B-lymphocytes and/or T-lymphocytes. An antigen is capable of inducing a humoral immune response and/or cellular immune response leading to the production of B- and/or T-lymphocytes. The structural aspect of an antigen that gives rise to a biological response is referred to herein as an “antigenic determinant.” B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors. An antigenic determinant need not be a contiguous sequence or segment of protein and may include various sequences that are not immediately adjacent to one another. In some embodiments, the antigen contains or is linked to a Th cell epitope. An antigen can have one or more epitopes (B-epitopes and T-epitopes). Antigens may also be mixtures of several individual antigens. Antigens can be any type of biologic molecule including, for example, simple intermediary metabolites, sugars, lipids, and hormones as well as macromolecules such as complex carbohydrates, phospholipids, nucleic acids and proteins. Common categories of antigens include, but are not limited to, viral antigens, bacterial antigens, fungal antigens, protozoa and other parasitic antigens, tumor antigens, antigens involved in autoimmune disease, allergy and graft rejection, and other miscellaneous antigens. Antigens can be microbial antigens, such as viral, fungal, or bacterial; or therapeutic antigens such as antigens associated with cancerous cells or growths, or autoimmune disorders. In some embodiments, the antigen is selected from a small molecule, nucleotide, polynucleotide, peptide, polypeptide, protein, lipid, carbohydrate, other immunogenic molecules, and a combination thereof. In some embodiments, the antigen is a bacterial antigen.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state or infection is known. The subject, or sample therefrom, may be healthy, diseased or infected, diseased or infected prior to treatment, diseased or infected during treatment, or diseased or infected after treatment, or a combination thereof.

“Identical” or “identity” as a percentage as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0

“Immunogenicity” refers to the ability of an antigen to induce an immune response and includes the intrinsic ability of an antigen to generate antibodies in a subject. In some embodiments, the self-assembling peptides described herein, or the nanofibers they form, are not immunogenic without an antigen appended thereto.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, mRNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. Secondary structure may include beta-sheet and alpha-helices. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. In some embodiments, a carrier includes a solution at neutral pH. In some embodiments, a carrier includes a salt. In some embodiments, a carrier includes a buffered solution. In some embodiments, a carrier includes phosphate buffered saline solution.

“Sample” or “test sample” as used herein can mean any sample in which the presence and/or level of a target is to be detected or determined or a portion from a subject or portion of an immunogenic composition as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease. A disease may include a bacterial infection. Bacterial infections may occur when bacteria enter the body of a subject, increase in number, and cause a reaction in the body. Bacteria can enter the body through an opening in the skin of a subject, such as a cut or surgical wound, or through orifices in the body of a subject, such as the airway (for example, nasal passages, mouth), urethra, ear canal, eye, and the like. A bacterial infection can be caused by either gram-negative or gram-positive bacteria. In some embodiments, the bacterial infection is caused by a uropathogenic bacteria. In certain embodiments, the uropathegenic bacteria comprises a uropathogenic E. coli bacteria.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto. A variant can be a polynucleotide sequence that is substantially identical over the full length of the full polynucleotide sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the polynucleotide sequence or a fragment thereof.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132, incorporated herein by reference). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the amino acid sequence or a fragment thereof.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. UPEC Conjugate Peptide

Provided herein is a Uropathogenic Escherichia coli (UPEC) conjugate peptide. The UPEC conjugate peptide may include a self-assembling peptide and at least one UPEC epitope attached thereto.

a. Self-Assembling Peptide

The compositions and methods detailed herein include self-assembling peptides. As used herein, the term “self-assembling peptide” refers to peptides that are able to spontaneously associate and form stable structures. Each self-assembling peptide may comprise or form an alpha helix. In other embodiments, each self-assembling peptide may comprise or form a beta-sheet. Examples of self-assembling peptides are detailed in, for example, U.S. Pat. Nos. 9,241,987; 9,849,174; 10,596,238; 11,246,924; International Patent Application Publication No. WO 2023/044163; Lee, S. et al. Int. J. Mol. Sci. 2019, 20, 5850; Hernandez, A. et al. Front. Bioeng. Biotechnol. 2023, 11, 1139782; and Lopez-Silva et al. ACS Biomater. Sci. Eng. 2019, 5, 977-985, each of which is incorporated herein by reference in its entirety.

In some embodiments, the self-assembling peptide comprises a polypeptide having an amino acid sequence selected from QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn), or a polypeptide with at least 75%, 80%, 85%, or 90% identity thereto. Some examples of self-assembling peptide are detailed in, for example, U.S. Pat. Nos. 9,241,987; 9,849,174; and 10,596,238, each of which is incorporated herein by reference in its entirety. In some embodiments, the self-assembling peptide comprises the amino acid sequence of QQKFQFQFEQQ (Q11, SEQ ID NO: 12), or a polypeptide with at least 75%, 80%, 85%, or 90% identity thereto. In some embodiments, the self-assembling peptide comprises the sequence Ac-QQKFQFQFEQQ-NH₂ (Q11, SEQ ID NO: 13), or a polypeptide with at least 75%, 80%, 85%, or 90% identity thereto.

The self-assembling peptide may comprise an amino acid sequence of bXXXb (SEQ ID NO: 1), wherein X is independently any amino acid, and b is independently any positively charged amino acid. In such embodiments, each self-assembling peptide may form an alpha helix. In some embodiments, b is independently selected from Arg and Lys. In some embodiments, b is Arg. In some embodiments, b is Lys. In some embodiments, bXXXb (SEQ ID NO: 1) is RAYAR (SEQ ID NO: 2). In some embodiments, bXXXb (SEQ ID NO: 1) is KAYAK (SEQ ID NO: 3). In some embodiments, the self-assembling peptide comprises the sequence of RXXXR (SEQ ID NO: 4), wherein X is any amino acid. The self-assembling peptide may comprise an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20. In some embodiments, n is an integer from 5 to 15, and m is an integer from 5 to 15. Some examples of self-assembling peptide are detailed in, for example, U.S. Pat. No. 11,246,924, which is incorporated herein by reference in its entirety. In such embodiments, a plurality of the UPEC conjugate peptides may assemble into a nanofiber.

In some embodiments, the self-assembling peptide comprises a glutamine at the C-terminus. In some embodiments, the self-assembling peptide comprises a glutamine at the N-terminus. The self-assembling peptide may include at least, at most, or exactly 5, 10, 15, 16, 17, 18, 19, 20, 21, 22, 23, 24, 25, 26, 27, 28, 29, 30, 31, 32, 33, 34, 35, or 40 amino acids. In some embodiments, the self-assembling peptide comprises 5 to 40 amino acids in length.

In some embodiments, the self-assembling peptide comprises an amino acid sequence of QARILEADAEILRAYARILEAHAEILRAQ (SEQ ID NO: 6) or QAKILEADAEILKAYAKILEAHAEILKAQ (SEQ ID NO: 7) or ADAEILRAYARILEAHAEILRAQ (SEQ ID NO: 8) or Ac-QARILEADAEILRAYARILEAHAEILRAQ-NH₂ (SEQ ID NO: 9) or Ac-QAKILEADAEILKAYAKILEAHAEILKAQ-NH₂ (SEQ ID NO: 10) or Ac-ADAEILRAYARILEAHAEILRAQ-NH₂ (SEQ ID NO: 11), or a polypeptide with at least 75%, 80%, 85%, 90%, or 95% identity thereto. In some embodiments, the self-assembling peptide comprises an amino acid sequence of QARILEADAEILRAYARILEAHAEILRAQ (SEQ ID NO: 6) or QAKILEADAEILKAYAKILEAHAEILKAQ (SEQ ID NO: 7) or ADAEILRAYARILEAHAEILRAQ (SEQ ID NO: 8) or a variant thereof. In some embodiments, the self-assembling peptide comprises an amino acid sequence of QARILEADAEILRAYARILEAHAEILRAQ (SEQ ID NO: 6). In some embodiments, the self-assembling peptide comprises an amino acid sequence of QAKILEADAEILKAYAKILEAHAEILKAQ (SEQ ID NO: 7). In some embodiments, the self-assembling peptide comprises an amino acid sequence of ADAEILRAYARILEAHAEILRAQ (SEQ ID NO: 8).

In some embodiments, each self-assembling peptide forms a beta sheet and comprises a polypeptide having an amino acid sequence selected from QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn), or a polypeptide with at least 75%, 80%, 85%, or 90% identity thereto. In such embodiments, a plurality of the UPEC conjugate peptides may assemble into a nanofiber.

In some embodiments, the self-assembling peptide comprises a polypeptide having an amino acid sequence selected from VEVKVEVKV (SEQ ID NO: 41), VEVKVEVKVEVK (SEQ ID NO: 42), VWVAAAEEE (SEQ ID NO: 43), VEVEVEVEVEVEVEVEVEVE (SEQ ID NO: 44), CGNKRTRGC (SEQ ID NO: 45), VKVKVKVKVDPPTKVEVKVKV (SEQ ID NO: 46), LRKKLGKA (SEQ ID NO: 47), WVVVVKK (SEQ ID NO: 48), AEAKAEAKAEAKAEAK (SEQ ID NO: 49), AEAKAEAK (SEQ ID NO: 50), AEAEAEAEAKAK (SEQ ID NO: 51), AEAEAKAK (SEQ ID NO: 52), AEAEAKAKAEAEAKAK (SEQ ID NO: 53), RADARADARADARADA (SEQ ID NO: 54), RADARGDARADARGDA (SEQ ID NO: 55), RADARADA (SEQ ID NO: 56), RARADADARARADADA (SEQ ID NO: 57), RARADADA (SEQ ID NO: 58), RARARARADADADADA (SEQ ID NO: 59), ADADADADARARARAR (SEQ ID NO: 60), DADADADARARARARA (SEQ ID NO: 61), RAEARAEARAEARAEA (SEQ ID NO: 62), RAEARAEA (SEQ ID NO: 63), KAKAKAKAEAEAEAEA (SEQ ID NO: 64), AEAEAEAEAKAKAKAK (SEQ ID NO: 65), KADAKADAKADAKADA (SEQ ID NO: 66), KADAKADA (SEQ ID NO: 67), AEAEAHAHAEAEAHAHA (SEQ ID NO: 68), AEAEAHAHA (SEQ ID NO: 69), HEHEHKHKHEHEHKHK (SEQ ID NO: 70), HEHEHKHK (SEQ ID NO: 71), FEFEFKFKFEFEFKFK (SEQ ID NO: 72), FEFKFEFK (SEQ ID NO: 73), LELELKLKLELELKLK (SEQ ID NO: 74), LELELKLK (SEQ ID NO: 75), KFDLKKDLKLDL (SEQ ID NO: 76), FKFEFKFF (SEQ ID NO: 77), FEFEFKFK (SEQ ID NO: 78), and RFRFRFRFRFRFRFRFRFRF (SEQ ID NO: 79), or a polypeptide with at least 75%, 80%, 85%, or 90% identity thereto. In such embodiments, a plurality of the PC-peptide conjugates may assemble into a nanofiber, nanotube, hydrogel, micelle, vesicle, nanoparticle, or suspension.

In some embodiments, the self-assembling polypeptide includes a modification to the C-terminus, to the N-terminus, or to both the C-terminus and N-terminus. N-terminal modifications may include, for example, biotin and acetyl. C-terminal modifications may include, for example, amide. In some embodiments, the N-terminus is acetylated (which may be indicated by “Ac” for example). In some embodiments, the C-terminus is amidated (which may be indicated by “NH₂” for example).

The peptides described herein can be chemically synthesized using standard chemical synthesis techniques. In some embodiments the peptides are chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides described herein. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc., 85:2149-2156; and Stewart et al. (1984) Solid Phase Peptide Synthesis, 2nd ed. Pierce Chem. Co., Rockford, III; each of which is incorporated herein by reference. In some embodiments, the self-assembling peptide is synthesized by a solid phase peptide synthesis.

b. Uropathogenic Escherichia coli (UPEC) Epitope

The UPEC conjugate peptide includes at least one UPEC epitope. The UPEC epitope may be a B-cell epitope. The UPEC epitope may be a B-cell epitope from uropathogenic E. coli. The UPEC epitope may be a B-cell epitope from an iron receptor protein from uropathogenic E. coli. The iron receptor protein may allow UPEC to survive in the iron-poor urinary tract. The iron receptor proteins may be surface-expressed, allowing for antibody binding. The iron receptor proteins may be selected from the proteins IreA, lutA, and IroN, or a combination thereof. The genes encoding these proteins were found in 34%, 66%, and 74% of clinical UPEC isolates, respectively. The UPEC epitope may be a fragment or segment from proteins IreA, lutA, and IroN, or a combination thereof. The UPEC epitope may be selected from plroN (YLLYSKGNGCPKDITSGGCYLIGNKDLDPE, SEQ ID NO: 80), plutA (VDDIDYTQQQKIAAGKAISADAIPGGSVD, SEQ ID NO: 81), or plreA (GIAKAFRAPSIREVSPGFGTLTQGGASIMYGN, SEQ ID NO: 82), or a combination thereof. The UPEC epitope may be selected from plroN (YLLYSKGNGCPKDITSGGCYLIGNKDLDPE, SEQ ID NO: 80), plutA (VDDIDYTQQQKIAAGKAISADAIPGGSVD, SEQ ID NO: 81), or plreA (GIAKAFRAPSIREVSPGFGTLTQGGASIMYGN, SEQ ID NO: 82), or a combination thereof, or a peptide having at least 80%, 81%, 82%, 83%, 84%, 85%, 86%, 87%, 88%, 89%, 90%, 91%, 92%, 93%, 94%, 95%, 96%, 97%, 98%, or 99% identity thereto. In some embodiments, the peptide fibril is coupled to a plurality of UPEC epitopes. UPEC epitopes may be obtained commercially or synthesized and purified by any suitable method known in the art.

The UPEC epitope may be conjugated or coupled to a self-assembling peptide by any means known in the art. The UPEC epitope may be covalently coupled to a terminus of the self-assembling peptide. At least one UPEC epitope may be attached to the C-terminus or the N-terminus of the self-assembling peptide. In some embodiments, the UPEC epitope is covalently coupled to the N-terminus or N-terminal end of the self-assembling peptide. In some embodiments, the UPEC epitope is covalently coupled to the C-terminus or C-terminal end of the self-assembling peptide. The conjugation of the UPEC epitope to the N-terminus or the N-terminal end of the self-assembling peptide may orient the UPEC epitope towards the exterior of the helical peptide fibril once a plurality of UPEC conjugate peptides assembles into a nanofiber. In some embodiments, the UPEC epitopes are exposed on the exterior surface of the nanofiber. In some embodiments, the UPEC epitopes are exposed on the exterior surface of the helical filament of the nanofiber.

The UPEC conjugate peptide may include at least one UPEC epitope. The UPEC conjugate peptide may include 1 to 10 UPEC epitopes attached to a self-assembling peptide. The UPEC conjugate peptide may include 1, 2, 3, 4, 5, 6, 7, 8, 9, or 10 UPEC epitopes attached to a self-assembling peptide. In embodiments including more than one UPEC epitope attached to a self-assembling peptide, all UPEC epitopes may be attached to the same end of the self-assembling peptide. For example, 1, 2, 3, or 4 UPEC epitopes together may be attached to the N-terminal end or to the C-terminal end of the self-assembling peptide. Once assembled into a nanofiber, the nanofiber may include n UPEC epitopes, wherein n is an integer from 1 to 1,000, or 1 to 5,000, or 1 to 10,000, or 1 to 50,000, or 1 to 100,000, including all values and ranges there between. The UPEC epitopes attached to a self-assembling peptide may be the same UPEC epitope or different UPEC epitopes.

In some embodiments, the antigens are exposed on the surface of the peptide fibril. In certain aspects, the ratio of antigen to self-assembling peptide is 1:1000, 1:100: 1:10, or 1:1, including all values and ranges there between.

c. Linker

The UPEC conjugate peptide may further comprise a linker. The linker may be between the UPEC epitope and the self-assembling peptide. The linker may be between the at least one UPEC epitope and the self-assembling peptide. In some embodiments, a linker is covalently attached to the self-assembling peptide between the UPEC epitope and the self-assembling peptide. In some embodiments, the linker comprises at least one cysteine. The at least one cysteine may be at the N-terminus of the linker. In some embodiments, the linker comprises glycine and serine. In some embodiments, the linker comprises glycine and serine and cysteine. In some embodiments, the UPEC epitope is attached to the self-assembling peptide through a thiol reactive group in the linker.

In some embodiments, the conjugate peptide includes more than one linker. In such embodiments, the linkers may be the same or different from one another. The conjugate peptide may include at least 1, at least 2, at least 3, at least 4, at least 5, at least 6, at least 7, at least 8, at least 9, or at least 10 linkers. The conjugate peptide may include less than 20, less than 15, less than 10, or less than 5 linkers. The conjugate peptide may include between 1 and 20, between 5 and 15, or between 1 and 5 linkers. The linker may be positioned at the C-terminus of the self-assembling peptide, at the N-terminus of the self-assembling peptide, or at both the N- and C-termini of the self-assembling peptide. In some embodiments, the linker is positioned at the N-terminus of the self-assembling peptide. Multiple linkers may be positioned adjacent to one another.

The linker may comprise, for example, an oligoethylene glycol, polyethylene glycol, or an amino acid sequence selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC)₈), SEQ ID NO: 92 ((G₄S)₃), SEQ ID NO: 93 (KSGSG), SEQ ID NO: 94 (KKSGSG), SEQ ID NO: 95 (EAAAK)₂, and SEQ ID NO: 96 (GGAAY). In some embodiments, the linker comprises (Ser-Gly)₂ (SEQ ID NO: 85).

d. PEG Molecule or PAS Peptide

The UPEC conjugate peptide may further comprise a polyethylene glycol (PEG) molecule. The PEG molecule may facilitate mucus penetration. The PEG molecule may be muco-inert. The PEG molecule may enable or facilitate oral availability. The PEG molecule may be an average molecular weight from 1000 Da and up to and including 100,000 Da, or from 1000 Da and up to and including 5,000 Da. The PEG molecule may comprise PEG2000, PEG1000, or PEG3000. In some embodiments, the PEG molecule comprises PEG2000. The PEG molecule may comprise CH₃O—(CH₂CH₂O)ₙ wherein n is an integer from about 1 to about 2000, or from about 1 to about 100, or from about 20 to about 70. In some embodiments, the average n is at least about 20, about 45, or less than about 70. A plurality of UPEC conjugate peptides may have the same or different PEG molecule attached to each self-assembling peptide. The PEG molecule may be conjugated to the self-assembling peptide at the N-terminus or N-terminal end or the C-terminus or C-terminal end. The PEG molecule may be conjugated to the self-assembling peptide at the opposite terminus from wherein the UPEC epitope is conjugated. The PEG molecule may be conjugated to the self-assembling peptide via a linker, as detailed above. The PEG molecule may be commercially obtained.

The UPEC conjugate peptide may further comprise a PAS peptide. The PAS peptide may be muco-inert. The PAS peptide may facilitate mucus penetration. The PAS peptide may enable or facilitate oral availability. The PAS peptide may comprise the amino acid sequence of Pro-Ala-Ser. The PAS peptide may comprise the amino acid sequence of ASPAAPAPASPAAPAPSAPA (SEQ ID NO: 97), or a peptide having at least 80%, 85%, 90%, or 95% identity thereto. In some embodiments, the PAS peptide comprises ASPAAPAPASPAAPAPSAPA (SEQ ID NO: 97). In some embodiments, the PAS peptide comprises H₂N-ASPAAPAPASPAAPAPSAPA-NH₂ (SEQ ID NO: 98). The PAS peptide may be conjugated to the self-assembling peptide at the N-terminus or N-terminal end or the C-terminus or C-terminal end. The PAS peptide may be conjugated to the self-assembling peptide at the opposite terminus from wherein the UPEC epitope is conjugated. The PAS peptide may be conjugated to the self-assembling peptide via a linker, as detailed above.

In some embodiments, the UPEC conjugate peptide does not include a PEG molecule or a PAS peptide. In some such embodiments, the self-assembling peptide and/or the UPEC epitope may have a hydrophobicity index such that the UPEC conjugate peptide has sufficient activity or effectiveness without a PEG molecule or a PAS peptide. In some embodiments, and as detailed in the Examples, a more hydrophobic epitope may need more of the conjugate peptides in a nanofiber to be PASylated or PEGylated to have a desired activity or effectiveness. In some embodiments, a more hydrophilic epitope and/or self-assembling peptide may not need any or may need less of the conjugate peptides in a nanofiber to be PASylated or PEGylated to have a desired activity or effectiveness.

The UPEC conjugate peptide may comprise, for example, PEG-Q11-plroN (SEQ ID NO: 101), PEG-Q11-plutA (SEQ ID NO: 102), or PEG-Q11-plreA (SEQ ID NO: 103), or a combination thereof. Exemplary UPEC conjugate peptides are shown in TABLE 1.

TABLE 1

| Name | Sequence | Function |
|---|---|---|
| Q11 | QQKFQFQFEQQ (SEQ ID NO: 12) | Self-assembly |
| | | into nanofibers |
| mPEG₂₀₀₀ | CH₃O-(CH₂CH₂O)ₙ Average n = 45 | Mucus |
| | | penetration |
| mPEG₂₀₀₀-plroN- | mPEG₂₀₀₀-SGSG-QQKFQFQFEQQ-SGSG- | UPEC B-cell |
| Q11 | YLLYSKGNGCPKDITSGGCYLIGNKDLDPE- | epitope |
| | NH₂ (SEQ ID NO: 101) | |
| mPEG₂₀₀₀-plutA- | mPEG₂₀₀₀-SGSG-QQKFQFQFEQQ-SGSG- | UPEC B-cell |
| Q11 | VDDIDYTQQQKIAAGKAISADAIPGGSVD-NH₂ | epitope |
| | (SEQ ID NO: 102) | |
| mPEG₂₀₀₀-plreA- | mPEG₂₀₀₀-SGSG-QQKFQFQFEQQ-SGSG- | UPEC B-cell |
| Q11 | GIAKAFRAPSIREVSPGFGTLTQGGASIMYGN- | epitope |
| | NH₂ (SEQ ID NO: 103) | |
| PADRE-Q11 | H₂N-aKXVAAWTLKAa-SGSG- | T-helper epitope |
| | QQKFQFQFEQQ-NH₂ (SEQ ID NO: 104) | |
| VAC-Q11 | H₂N-QLVFNSISARALKAY-SGSG- | T-helper epitope |
| | QQKFQFQFEQQ-NH₂ (SEQ ID NO: 105) | |

### 3. T-Cell Epitope Conjugate Peptide

Further provided herein is a T-cell epitope conjugate peptide. The T-cell epitope conjugate peptide may include a self-assembling peptide and at least one T-cell epitope attached thereto. The self-assembling peptide of the T-cell epitope conjugate peptide may be as detailed above for the UPEC conjugate peptide. The T-cell epitope may be selected from PADRE and VAC. The PADRE molecule may comprise a polypeptide having the amino acid sequence of aKXVAAWTLKAa, wherein “X” comprises cyclohexylalanine and “a” comprises D-alanine (SEQ ID NO: 99). In some embodiments, the cyclohexylalanine comprises D-alanine. The VAC molecule may comprise a polypeptide having the amino acid sequence of QLVFNSISARALKAY (SEQ ID NO: 100).

In some embodiments, the T-cell epitope conjugate peptide further comprises a linker between the T-cell epitope and the self-assembling peptide. The linker of the T-cell epitope conjugate peptide may be as detailed above for the UPEC conjugate peptide.

The PADRE-conjugate peptide may comprise, for example, the sequence of NH₂-aKXVAAWTLKAa-SGSG-QQKFQFQFEQQ-NH₂ (PADRE-Q11, SEQ ID NO: 104) or aKXVAAWTLKAa-SGSG-QQKFQFQFEQQ (SEQ ID NO: 106). The VAC-conjugate peptide may comprise, for example, the sequence of NH₂-QLVFNSISARALKAY-SGSG-QQKFQFQFEQQ-NH₂ (VAC-Q11, SEQ ID NO: 105) or QLVFNSISARALKAY-SGSG-QQKFQFQFEQQ (SEQ ID NO: 107).

### 4. Plain Self-Assembling Peptides

Some embodiments include plain self-assembling peptides, which refers to a self-assembling peptide as detailed above, but without any antigen such as a UPEC epitope or a T-cell epitope (such as PADRE or VAC) attached thereto.

### 5. Nanofibers

Further described herein is a platform for vaccination or treatment based on peptides assembled into nanofibers. The nanofiber may also be referred to as a peptide fibril. The nanofibers may be comprised of alpha-helical peptides. The nanofibers may be comprised of beta-sheet peptides. In this strategy, peptides fold into a complex beta-sheet-based or alpha-helix-based nanofiber where individual peptide coils run perpendicular to the axis of a long fibril. Each self-assembling peptide may comprise or form an alpha helix. The plurality of self-assembling peptides may form a peptide fibril in the form of a helical filament. The resultant nanostructure is composed of thousands of individual peptides or more. The self-assembling peptide may be extended N-terminally with a flexible spacer and an immune epitope such as a UPEC epitope. In some embodiments, the nanofiber does not further comprise an adjuvant. In some embodiments, the nanofiber is an adjuvant.

Multiple epitope-bearing self-assembling peptides may be co-assembled into nanofibers composed of β-sheets or α-helices. Coiled coil folding requires more extensive design considerations compared to β-sheet fibrillization, as both inter-helical interactions as well as those between the C-terminus and the main chain must be considered.

The nanofiber may be comprised of 10 to 10,000 peptides including all values and ranges there between. The nanofiber may include a combination of 10 to 10,000, or 100 to 10,000 peptides comprising UPEC conjugate peptides and T-cell epitope conjugate peptides. The nanofiber may comprise a combination of 10 to 10,000, or 100 to 10,000 peptides comprising UPEC conjugate peptides, T-cell epitope conjugate peptides, and plain self-assembling peptides.

The nanofiber may include at least one UPEC conjugate peptide. The nanofiber may include a plurality of UPEC conjugate peptides. The nanofiber may include at least 10, 20, 30, 40, 50, 60, 70, 80, 90, 100, 150, 200, 250, 300, 400, 450, 500, 550, 600, 650, 700, 750, 800, 850, 900, 950, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, or 9000, or less than 10,000 of the same or different UPEC conjugate peptides. The UPEC conjugate peptides making up a single nanofiber may be the same or different. The nanofiber may further include at least one T-cell epitope conjugate peptide. The nanofiber may include a plurality of T-cell epitope conjugate peptides. The nanofiber may include at least 1, 5, 10, 20, 30, 40, 50, 60, 70, 80, 90, 100, 150, 200, 250, 300, 400, 450, 500, 550, 600, 650, 700, 750, 800, 850, 900, 950, 1000, 1200, 1400, 1600, 1800, 2000, 2200, 2400, 2600, 2800, 3000, 3200, 3400, 3600, 3800, 4000, 4200, 4400, 4600, 4800, or 5000 of the same or different T-cell epitope conjugate peptides. The T-cell epitope conjugate peptides making up a single nanofiber may be the same or different. In some embodiments, the nanofiber includes self-assembling peptides without a T-cell epitope or without a UPEC epitope conjugated thereto, which may be referred to as a plain self-assembling peptide. The nanofiber may further include at least one plain self-assembling peptide. The nanofiber may include a plurality of plain self-assembling peptides. The nanofiber may include at least 10, 20, 30, 40, 50, 60, 70, 80, 90, 100, 150, 200, 250, 300, 400, 450, 500, 550, 600, 650, 700, 750, 800, 850, 900, 950, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, or 9000, or less than 10,000 of the same or different plain self-assembling peptides. The plain self-assembling peptides making up a single nanofiber may be the same or different.

A single nanofiber may include different peptides in a variety of ratios. The nanofiber may include a variety of ratios of UPEC conjugate peptides to T-cell epitope conjugate peptides. In some embodiments, at least about 1%, 5%, 10%, 15%, 20%, 25%, 30%, 35%, 40%, 45%, 50%, 55%, 60%, 65%, 70%, 75%, 80%, 85%, 90%, 95%, or 97.5% of the peptides in the nanofiber are UPEC conjugate peptides. In some embodiments, at least about 1%, 2%, 3%, 4%, 5%, 6%, 7%, 8%, 9%, 10%, 11%, 12%, 13%, 14%, or 15% of the peptides in the nanofiber are T-cell epitope conjugate peptides. In some embodiments, at least about 1%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 92.5%, 95%, or 96.5%, or less than about 96.5% of the peptides in the nanofiber are plain self-assembling peptides. In some embodiments, the UPEC peptide conjugate and the T-cell epitope peptide conjugate are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1.

The helical filament of the nanofiber may be formed around a central axis or core. The plurality of self-assembling peptides may form a peptide fibril in the form of a coiled coil. In some embodiments, the N-terminus of each self-assembling peptide is positioned at the exterior of the helical filament. The UPEC epitopes may be exposed on the exterior surface of the nanofiber. The T-cell epitopes may be exposed on the exterior surface of the nanofiber. An example of the self-assembling peptides formed into a peptide fibril is shown schematically in Egelman et al. (Structure 2015, 23, 280-289, incorporated herein by reference).

Nanofibers have been observed to be up to several microns long. The nanofiber can have a length of at least, at most, or exactly 100 nm, 200 nm, 300 nm, 400 nm, 500 nm, 600 nm, 700 nm, 800 nm, 900 nm, 1 μm, 1.2 μm, 1.4 μm, 1.6 μm, 1.8 μm, 2 μm, 2.2 μm, 2.4 μm, 2.6 μm, 2.8 μm, 3 μm, 3.2 μm, 3.4 μm, 3.6 μm, 3.8 μm, 4 μm, 4.2 μm, 4.4 μm, 4.6 μm, 4.8 μm, or 5 μm. The nanofiber may be about 100 nm to 1 μm, 100 nm to 2 μm, 100 nm to 3 μm, 100 nm to 4 μm, or 100 nm to 5 μm in length. The nanofiber can have a length of at least, at most, or exactly 0.01, 0.05, 0.1, 0.15, 0.20, 0.25, 0.5, 1, 5, 10, 20, 30, 40, 50, 75, 100, 125, 150, 175, 200, 225, 250, or 300 μm, including all values and ranges there between. In some embodiments, the nanofiber is at least 100, 150, 200, 250, 300, or 350 nanometers in length. In some embodiments, the nanofiber is less than 10, 5, or 2 μm in length. In some embodiments, the nanofiber is 50 nm to 600 nm in length. In certain aspects, the nanofiber has a molecular weight of at least 100, 500, 1,000, 5,000, 10,000, 100,000 Da to 1×10⁶, 1×10⁷, 7×10⁸ Da, including all values and ranges there between. The nanofiber can have a diameter or width of at least, at most, or exactly 5, 10, 15, 20, 25, or 30 nm. In some embodiments, the nanofiber is 5-30 nm in diameter or width.

### 6. Immune Response and Immunoassays

As discussed above, the compositions and methods provided herein include evoking or inducing an immune response in a subject against an antigen. The antigen may comprise a UPEC epitope. In one embodiment, the immune response can protect against or treat a subject having, suspected of having, or at risk of developing an infection or related disease. One use of the immunogenic compositions is to provide effective vaccines. The compositions detailed herein may induce an immune response. The immune response may be an antigen-specific immune response. In some embodiments, the antigen-specific immune response is temporary or not life-long. The anti-UPEC antibodies generated may be IgM, or IgG, or IgA, or a combination thereof. In some embodiments, the antibodies are IgM antibodies. The immune response may include IgG antibody isotypes and subclasses. In some embodiments, the immune response comprises IgG1, IgG2, IgG3, or IgG4 antibody isotypes, or a combination thereof. In some embodiments, the immune response comprises IgM antibody isotypes. The immunogenic composition may have increased immunogenicity relative to a control. In some embodiments, the control comprises a UPEC epitope or other antigen without a self-assembling peptide.

The compositions and methods detailed herein may increase the level of anti-UPEC antibodies relative to a control. The level of anti-UPEC antibodies may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The level of anti-UPEC antibodies may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The level of anti-UPEC antibodies may be increased by about 5-95%, 10-90%, 15-85%, 20-80%, or 1.5-fold to 10-fold, relative to a control.

Further provided herein is the implementation of serological assays to evaluate whether and to what extent an immune response is induced or evoked by compositions. There are many types of immunoassays that can be implemented. Immunoassays include, but are not limited to, those described in U.S. Pat. No. 4,367,110 (double monoclonal antibody sandwich assay) and U.S. Pat. No. 4,452,901 (western blot), which are incorporated herein by reference. Other assays include immunoprecipitation of labeled ligands and immunocytochemistry, both in vitro and in vivo.

Immunoassays generally are binding assays. Certain immunoassays are the various types of enzyme linked immunosorbent assays (ELISAs) and radioimmunoassays (RIA) known in the art. Immunohistochemical detection using tissue sections may also be useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

a. Protective Immunity

In some embodiments, proteinaceous compositions confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein the phrase “immune response” or its equivalent “immunological response” may refer to the development of a humoral (antibody mediated), cellular (mediated by antigen-specific T cells or their secretion products) or both humoral and cellular response directed against a protein, peptide, carbohydrate, or polypeptide in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject by administration of an antigen.

As used herein “passive immunity” refers to any immunity conferred upon a subject without administration of an antigen to the subject. “Passive immunity” therefore includes, but is not limited to, administration of activated immune effectors including cellular mediators or protein mediators (e.g., monoclonal and/or polyclonal antibodies) of an immune response. A monoclonal or polyclonal antibody composition may be used in passive immunization for the prevention or treatment of infection by organisms that carry the antigen recognized by the antibody. An antibody composition may include antibodies that bind to a variety of antigens that may in turn be associated with various organisms. The antibody component can be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as gram-positive bacteria, gram-negative bacteria, including but not limited to Staphylococcus bacteria or Escherichia bacteria.

Passive immunity may be imparted to a patient or subject by administering to the patient immunoglobulins (lg) and/or other immune factors obtained from a donor or other non-patient source having a known immunoreactivity. In other aspects, an antigenic composition as detailed herein can be administered to a subject who then acts as a source or donor for globulin, produced in response to challenge with the antigenic composition (“hyperimmune globulin”), that contains antibodies directed against Staphylococcus or Escherichia or other organism, for example. A subject thus treated would donate plasma from which hyperimmune globulin would then be obtained, via conventional plasma-fractionation methodology, and administered to another subject in order to impart resistance against or to treat Staphylococcus or Escherichia infection. Hyperimmune globulins are particularly useful for immune-compromised individuals, for individuals undergoing invasive procedures or where time does not permit the individual to produce their own antibodies in response to vaccination. See U.S. Pat. Nos. 6,936,258, 6,770,278, 6,756,361, 5,548,066, 5,512,282, 4,338,298, and 4,748,018, each of which is incorporated herein by reference in its entirety, for exemplary methods and compositions related to passive immunity.

For purposes of this specification and the accompanying claims the terms “epitope” and “antigenic determinant” are used interchangeably to refer to a site on an antigen to which B and/or T cells respond or recognize. B-cell epitopes can be formed from small molecules. For example, B-cell epitopes may be formed from a UPEC epitope. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996), incorporated herein by reference. Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., 1994, incorporated herein by reference), by antigen-dependent killing (cytotoxic T lymphocyte assay, Tigges et al., 1996, incorporated herein by reference) or by cytokine secretion.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

As used herein and in the claims, the terms “antibody” or “immunoglobulin” are used interchangeably and refer to any of several classes of structurally related proteins that function as part of the immune response of an animal or recipient, which proteins include IgG, IgD, IgE, IgA, IgM, and related proteins.

Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains.

In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific. Monoclonal antibodies can be produced by hyperimmunization of an appropriate donor with the antigen or ex-vivo by use of primary cultures of splenic cells or cell lines derived from spleen (Anavi, 1998; Huston et al., 1991; Johnson et al., 1991; Mernaugh et al., 1995, each incorporated herein by reference).

As used herein, the phrase “an immunological portion of an antibody” includes a Fab fragment of an antibody, a Fv fragment of an antibody, a heavy chain of an antibody, a light chain of an antibody, a heterodimer consisting of a heavy chain and a light chain of an antibody, a variable fragment of a light chain of an antibody, a variable fragment of a heavy chain of an antibody, and a single chain variant of an antibody, which is also known as scFv. In addition, the term includes chimeric immunoglobulins which are the expression products of fused genes derived from different species, one of the species can be a human, in which case a chimeric immunoglobulin is said to be humanized. Typically, an immunological portion of an antibody competes with the intact antibody from which it was derived for specific binding to an antigen.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

### 7. Pharmaceutical Compositions

Further provided herein are pharmaceutical compositions comprising the UPEC peptide conjugate or nanofibers detailed herein. Compositions can include a peptide fibril coupled to a plurality of antigens such as a UPEC epitope, and may be referred to as a “fibril complex.” In some embodiments, the composition does not further comprise an adjuvant. In some embodiments, the composition further comprises an adjuvant. In some embodiments, the peptide fibril is an adjuvant.

The preparation of pharmaceutical compositions such as vaccines that contain polypeptide or peptide sequence(s) as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such pharmaceutical compositions are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, pharmaceutical compositions are formulated with a combination of substances, as described in U.S. Pat. Nos. 6,793,923 and 6,733,754, which are incorporated herein by reference.

Pharmaceutical compositions may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

In some embodiments, the compositions described herein are formulated for sublingual administration. The compositions may be formulated into a tablet. For example, the compositions may be formulation into a dissolving tablet. The dissolving tablet may be placed under the tongue. The compositions may be formulated into a table by a variety of suitable means, for example, the compositions may be freeze dried and pressed into a tablet form.

The compositions described herein may be formulated into a pharmaceutical composition as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Typically, compositions are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection and the like. The dosage of the vaccine will depend on the route of administration and will vary according to the size and health of the subject.

The compositions and related methods, particularly administration of a peptide conjugate or nanofiber, may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline or various combinations of antibiotics.

In one aspect, it is contemplated that a peptide conjugate or nanofiber or pharmaceutical composition is used in conjunction with an additional treatment described herein. Alternatively, the therapy may precede or follow the other agent treatment by intervals ranging from minutes to weeks. In embodiments where the other agents and/or a proteins is administered separately, one may generally ensure that a significant period of time did not expire between the time of each delivery, such that the agent and antigenic composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6, or 7) to several weeks (1, 2, 3, 4, 5, 6, 7, or 8) lapse between the respective administrations.

Various combinations may be employed, for example antibiotic therapy is “A” and the immunogenic composition is “B”:

A/B/A B/A/B B/B/A A/A/B A/B/B B/A/A A/B/B/B
B/A/B/B B/B/B/A B/B/A/B A/A/B/B A/B/A/B
A/B/B/A B/B/A/A B/A/B/A B/A/A/B A/A/A/B B/A/A/A
A/B/A/A A/A/B/A

Administration of the pharmaceutical compositions to a patient/subject will follow general protocols for the administration of such compounds, taking into account the toxicity, if any. It is expected that the treatment cycles would be repeated as necessary. It also is contemplated that various standard therapies, such as hydration, may be applied in combination with the described therapy.

In some embodiments, pharmaceutical compositions are administered to a subject. Different aspects involve administering an effective amount of a composition to a subject. In some embodiments, immunogenic compositions may be administered to the patient to protect against infection by one or more microbial pathogens. Additionally, such compounds can be administered in combination with an antibiotic or other known anti-microbial therapy. Such compositions will generally be dissolved or dispersed in a pharmaceutically acceptable carrier or aqueous medium. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-cancer agents, can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including creams, lotions, mouthwashes, inhalants and the like.

The active compounds can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Administration of the compositions will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients. In some embodiments, the composition is administered to the subject intravenously, intraarterially, intraperitoneally, subcutaneously, intranasally, intramuscularly, or intratumorally. In some embodiments, the immunogenic composition is administered orally. In some embodiments, the immunogenic composition is administered sublingually.

For parenteral administration in an aqueous solution, for example, the solution may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions may be administered in a manner compatible with the dosage formulation and in such amount as is therapeutically or prophylactically effective. The formulations may be easily administered in a variety of dosage forms, such as the type of injectable solutions described above.

In some embodiments, the pharmaceutical compositions comprising the UPEC peptide conjugate or nanofibers detailed herein further include at least one additional therapeutic agent. The at least one additional therapeutic agent may comprise antibiotics, NSAIDS, anti-inflammatory compounds, hemoperfusion devices, quorum sensing inhibitors, lytic bacteriophage, polyclonal or monoclonal antibodies, non-immune tolerizing approaches, liposome-based cytotoxin inhibitors, or combinations thereof. The at least one additional therapeutic agent may comprise an adjuvant. In some embodiments, the adjuvant comprises CpG, cholera toxin B subunit (CTB), STING agonists like cyclic dinucleotides such as cyclic-di-AMP and/or cyclic GMP-AMP (cGAMP), retinoic acid, or heat labile toxin B subunit, or a combination thereof.

### 8. Methods

a. Methods of Treating a Urinary Tract Infection (UTI)

Further provided herein are methods of treating a urinary tract infection (UTI). Further provided herein are methods of treating or reducing a bacterial infection. Further provided herein are methods of treating or reducing a uropathogenic Escherichia coli infection. The methods may include administering to the subject a UPEC conjugate peptide, or nanofiber, or pharmaceutical composition as detailed herein. The UPEC conjugate peptide, or nanofiber, or pharmaceutical composition may be administered intraperitoneally, orally, sublingually, intravenously, nasally, buccally, transdermally, intranasally, or intramuscularly. In some embodiments, the UPEC conjugate peptide, or nanofiber, or pharmaceutical composition is administered sublingually.

In some embodiments, method further comprises administering at least one additional therapeutic agent. The at least one additional therapeutic agent may be administered prior to the UPEC conjugate peptide or the nanofiber or the pharmaceutical composition, or concurrently with the UPEC conjugate peptide or the nanofiber or the pharmaceutical composition, or after the UPEC conjugate peptide or the nanofiber or the pharmaceutical composition. The at least one additional therapeutic agent may comprise antibiotics, NSAIDS, anti-inflammatory compounds, hemoperfusion devices, quorum sensing inhibitors, lytic bacteriophage, polyclonal or monoclonal antibodies, non-immune tolerizing approaches, liposome-based cytotoxin inhibitors, or combinations thereof. The at least one additional therapeutic agent may comprise an adjuvant. In some embodiments, the adjuvant comprises CpG, cholera toxin B subunit (CTB), STING agonists like cyclic dinucleotides such as cyclic-di-AMP and/or cyclic GMP-AMP (cGAMP), retinoic acid, or heat labile toxin B subunit, or a combination thereof.

Upon or after administration, a bacterial infection, such as in the urinary tract, may be treated or reduced. The bacteria may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The bacteria may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The bacteria be reduced by about 5-95%, 10-90%, 15-85%, 20-80%, or 1.5-fold to 10-fold, relative to a control. The bacterial infection may be caused by uropathogenic bacteria. The uropathogenic bacteria may comprise uropathogenic E. coli. Upon or after administration, pathogenic bacteria, for example, in the urinary tract, may be reduced. The pathogenic bacteria may comprise CFT073. Upon or after administration, non-pathogenic bacteria may not be reduced. Upon or after administration, the microbiome in the gastrointestinal tract may not be reduced. Bacterial diversity in the colon may not be reduced. Bacteria in the gut may include, for example, bacteria from the genus Corynebacterium, Bifodobacterium, Atopobium, Faecalibacterium, Clostridium, Roseburia, Ruminococcus, Dialister, Lactobacillus, Enterococcus, Staphylococcus, Sphingobacterium, Bacteroides, Tannerella, Parabacteroides, Alistipes, Prevotella, Escherichia, Shigella, Desulfovibrio, Bilophila, Helicobacter, Fusobacterium, and Akkermansia, or a combination thereof. Bacteria in the gut may include, for example, bacteria from the species Bifodobacterium longum, Bifodobacterium bifidum, Faecalibacterium prausnitzii, Clostridium spp., Roseburia intestinalis, Ruminococcus faecis, Dialister invisus, Lactobacillus reuteri, Enterococcus faecium, Staphylococcus leei, Bacteroides fragilis, Bacteroides vulgatus, Bacteroides uniformis, Parabacteroides distasonis, Alistipes finegoldii, Prevotella spp., Escherichia coli, Shigella flexneri, Desulfovibrio intestinalis, Bilophila wadsworthia, Helicobacter pylori, Fusobacterium nucleatum, and Akkermansia muciniphila, or a combination thereof. The microbiome in the colon may be maintained. In some embodiments, the microbiome in the colon statistically maintains a Shannon Diversity Index.

b. Methods of Inducing an Immune Response

Further provided herein are methods of inducing an immune response in a subject. The methods may include administering to the subject a UPEC conjugate peptide, or nanofiber, or pharmaceutical composition as detailed herein. In some embodiments, the B1a cells in the subject express antibodies upon or subsequent to administration. The antibodies may be selected from IgM, IgG, and IgA, or a combination thereof. Further provided herein is an antibody produced in the immune response. In some embodiments, the UPEC peptide conjugate or the nanofiber is administered to the subject intraperitoneally, orally, sublingually, intravenously, nasally, buccally, transdermally, intranasally, or intramuscularly. In some embodiments, the UPEC conjugate peptide, or nanofiber, or pharmaceutical composition is administered sublingually. In some embodiments, method further comprises administering at least one additional therapeutic agent. The at least one additional therapeutic agent may be administered prior to the UPEC conjugate peptide or the nanofiber or the pharmaceutical composition, or concurrently with the UPEC conjugate peptide or the nanofiber or the pharmaceutical composition, or after the UPEC conjugate peptide or the nanofiber or the pharmaceutical composition. The at least one additional therapeutic agent may comprise an adjuvant. In some embodiments, the adjuvant comprises CpG, cholera toxin B subunit (CTB), STING agonists like cyclic dinucleotides such as cyclic-di-AMP and/or cyclic GMP-AMP (cGAMP), retinoic acid, or heat labile toxin B subunit, or a combination thereof.

### 9. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Materials and Methods

Peptide-Polymer Synthesis and Nanofiber Preparation. Peptides were synthesized using standard Fmoc solid-phase synthesis on Rink amide resins. PEG-peptides were synthesized by on-resin conjugation of 2000 MW mPEG-NHS (Creative PEGWorks PLS-214) to the N-terminus. Peptides were cleaved for 2 h at room temperature in a 95/2.5/2.5 TFA/triisopropylsilane/water cocktail, followed by washing with cold diethyl ether. Peptides were purified by reverse-phase HPLC using a C4 column (PEG-peptides) or C18 column (non-PEGylated peptides) and lyophilized. Peptide identity was confirmed using matrix-assisted laser desorption/ionization mass spectrometry (MALDI) on a Bruker Autoflex Speed LRF MALDI-TOF spectrometer using α-cyano-4-hydroxycinnamic acid (Sigma Aldrich, 70990) as the matrix.

To prepare nanofiber solutions, lyophilized peptides were dissolved at 8 mM in sterile water and incubated at 4° C. overnight. The solutions were then brought to the final concentrations in 1×PBS by addition of sterile water and sterile 10×PBS and incubated at room temperature for 3 h to before use to allow for fibrillization. Co-assembled nanofibers were prepared by vortexing lyophilized B-cell epitope peptides (PEG-Q11plreA, PEG-Q11plutA, PEG-Q11plroN) for 20 minutes and dissolving in a VACQ11 or Q11 solution at 8 mM total peptide. For multiple nanofiber formulations for immunization, each nanofiber was separately assembled and combined just prior to immunization.

For adjuvanted solutions, nucleotide adjuvants (cyclic-di-AMP, CpG) were added just prior to fibrillization; all other adjuvants were added after fibrillization. Adjuvants were included in formulations at the following dosages per mouse: 10 μg cholera toxin B subunit (List Labs, 104), 10 μg cyclic-di-AMP (Invivogen, vac-nacda), 25 μg CpG (Invivogen, tlrl-1826), 20 μg CRX-527 (Invivogen, tlrl-crx527), 15 μg compound 48/80 (Millipore Sigma, C2313).

Nanofiber Characterization by Electron Microscopy. To visualize nanofiber morphology by transmission electron microscopy, nanofiber solutions were diluted to 0.2 mM in 1×PBS and deposited onto Formvar/carbon-coated 400 mesh copper grids (Electron Microscopy Sciences, EMS400-Cu) for 1 min, rinsed with ultrapure water, and negatively stained for 1 min with 1% w/v uranyl acetate (EMS, 22400-1) prior to wicking away with filter paper. Samples were imaged on an FEI Tecnai G² Twin electron microscope at 120 kV.

Tablet Production Process. Reverse tablet molds were designed in FreeCAD and 3D-printed with a MakerBot Ultimaker 3. PDMS molds were prepared using SYLGARD 184 kits (Sigma, 761028). Fibrillized nanofiber solutions were mixed with adjuvants and sugars to a final concentration of 7.8 wt % each of trehalose (Santa Cruz Biotechnology, 394303), dextran (Alfa Aesar, J61216), and mannitol (Millipore Sigma, M4125). Final solutions were pipetted into the PDMS tray (30 μL per tablet), frozen at −80° C., and lyophilized (Kelly, et al. Advanced Healthcare Materials 2021, 10, 2001614, incorporated herein by reference).

Estimation of Clinical Tablet Dissolution Time. Five immunization-formulation tablets containing PEG-Q11 (PlreA/PlutA/PlroN/VAC) were individually tested. For each tablet, 1.0 mL of pooled human saliva (Innovative Research, IRHUSL) was warmed to 37° C. and dispersed on a petri dish. A tablet was dropped atop the saliva and the dissolution time was measured, using USP guidelines to determine the point of disintegration (https://www.usp.org/sites/default/files/usp/document/harmonization/gen-chapter/april-2019-m99460.pdf). A previous study showed 1.0 mL to be the approximate volume of saliva in a human mouth prior to swallowing.

Animals and Immunizations. Animal experiments were approved by the Institutional Care and Use Committee of Duke University under protocol #A264-18-11. Murine immunizations were initiated with female C57BL/6 mice (Envigo) aged 9-12 weeks. For sublingual immunizations, mice were deeply anesthetized by a cocktail delivering 100 mg/kg ketamine and 10 mg/kg xylazine. For droplet immunizations, a micropipette with a 20 μL tip was used to apply 8 μL of the immunizing solution below the tongue. For tablet immunizations, the tablet was placed below the tongue of anesthetized mice using tweezers. For droplet and tablet immunizations, the mouse's heads were placed in anteflexion for 20 min following administration to prevent swallowing of the material.

Rabbit immunizations were conducted with 8 month old female New Zealand White rabbits (Envigo). Rabbits were anesthetized with 20 mg/kg ketamine and 3 mg/kg xylazine. Tablets were placed under the rabbits' tongue using tweezers, rabbits' head were placed in anteflexion for 20 minutes, and recovery was initiated by atipamezole (xylazine reversal agent).

The total peptide concentration was 5 mM for all immunizations. For single epitope immunizations with PADRE, the B-cell epitope was 4.75 mM and PADRE was 0.25 mM, unless indicated otherwise. For single-epitope immunizations with VAC, the B-cell epitope was 4.4 mM and VAC was 0.6 mM. For co-assembled multi-B cell epitope nanofibers (including tablets in both mice and rabbits), each B-cell epitope was 1.4 mM and VAC was 0.6 mM. For mixed nanofiber formulations, each B-cell epitope was at 3× the final concentration, to keep total epitope dose consistent with single nanofiber formulations after mixing. For no VAC tablets, VACQ11 was replaced with the same concentration of unmodified Q11 to keep the total peptide concentration constant.

Measurement of Antibody Responses. Serum was collected via the submandibular vein. For analysis of epitope-specific IgG by ELISA, plates were coated with 20 μg/mL of streptavidin (Millipore Sigma, 189730) overnight at 4° C. Plates were washed with 0.5 g/L Tween-20 in PBS (1×PBST), coated for 1 hour with 20 μg/mL biotinylated plreA, pluta, or plroN, and blocked with Super Block Blocking Buffer (Thermo Scientific, 37515). When indicated in figure legends, plates were coated with a 1:1:1 mixture of biotinylated plreA, plutA, and plroN, at a total concentration of 20 μg/mL. Sera diluted in PBST-BSA or undiluted urine were added to the plate, and antigen-specific IgG was detected by horseradish peroxidase (HRP) conjugated Fcγ fragment specific goat anti-mouse IgG (Jackson Immuno Research, 15-035-071). Results are reported as antibody titers calculated with an absorbance cutoff of 0.2 OD, or as background-subtracted A450 values. For measuring antibody subclasses, HRP conjugated goat anti-mouse detection antibodies were purchased from Southern Biotech (IgG1: 1071-05, IgG2b: 1091-05, IgG2c: 1078-05). For whole-cell ELISAs, bacteria were cultured as indicated in the following section and plates were coated with an OD₆₀₀=1.0 solution in 1×PBS overnight and blocked for 2 h with 10% heat-inactivated fetal bovine serum in 1×PBS prior to addition of sera. Total (non antigen-specific) IgA was determined using a mouse IgA ELISA kit (Thermo Scientific, 88-50450).

Bacterial Culture and Challenge Model. The uropathogenic E. coli strain CFT073 was purchased from ATCC (700928); this strain was originally isolated from a pyelonephritis patient. CFT073 was cultured at 37° C. with aeration in Luria broth (10 g/L tryptone, 5 g/L yeast extract, 10 g/L sodium chloride) at a 1:100 dilution from a starter culture. For iron-limited culture conditions, the chelator 2,2′-bipyridyl (Millipore Sigma, D216305) was present at 400 μM in Luria broth. The non-uropathogenic E. coli strain BL21 was purchased from New England BioLabs (C2530H) and cultured was performed as with CFT073, but with the use of terrific broth (Fisher Scientific, BP2468).

For the bacteremia model, immunized or naive mice were challenged with intraperitoneal injection of 1×10⁸ or 2×10⁸ CFU of CFT073 in 1×PBS, as specified in figure captions. Temperature and body weight of mice were recorded at regular intervals for 72 h. Humane endpoints were set as a temperature below 32° C., loss of 25% of body weight, or exhibition of significant clinical symptoms, as prescribed by IACUC guidelines.

For the transurethral infection model, water supply was removed from cages for one hour prior to infection to allow for voiding of bladder. Mice were sedated with an i.p. injection of a ketamine/xylazine cocktail and a sterilized plastic needle dabbed in surgical lubricant was used to deliver 5×10⁷ CFU of CFT073 into the urethra. Mice were monitored as for the bacteremia model, but with sacrifice at 48 h.

Microbiome Analysis. Fecal pellets were collected from mice and immediately frozen on dry ice prior to storage at −80° C. Samples were submitted to the Duke Center for Genomic and Computational Biology Microbiome Shared Resource core for microbiome analyses. In brief, the Duke Microbiome Core Facility (MCF) extracted DNA using the Qiagen PowerSoilPro DNA Kit (Qiagen, 47014). Bacterial community composition in isolated DNA samples was characterized by amplification of the V4 variable region of the 16S rRNA gene by polymerase chain reaction using the forward primer 515 and reverse primer 806 following the Earth Microbiome Project protocol (http://www.earthmicrobiome.org/). These primers (515F and 806R) carry unique barcodes that allow for multiplexed sequencing. Concentration of the PCR products were accessed using a Qubit dsDNA HS assay kit (ThermoFisher, Q32854) and a Promega GloMax plate reader. Equimolar 16S rRNA PCR products from all samples were pooled prior to sequencing, which was performed by the Duke Sequencing and Genomic Technologies (SGT) shared resource on an Illumina MiSeq instrument configured for 250 base-pair paired-end sequencing runs.

Sequences were analyzed using QIIME2 software. Demultiplexed forward reads were assigned a cut off between 10-230 bp and reverse reads were assigned a cut off between 10-160 bp. Samples were trimmed to a depth of 94132 to retain 6,212,712 (73.67%) features in 66 (94.29%) samples. This sampling depth was used to rarefy the feature table to calculate alpha and beta diversity via a phylogenic rooted tree. Alpha rarefaction was plotted to confirm the richness of the samples had been fully observed. Sequences were then classified against the Greengenes 13_8 99% OTUs full-length sequences training set for taxonomic analysis.

### Example 2

### Selection of B-Cell Epitopes from Uropathogenic E. coli

Human UTIs can be caused by several genera of bacteria, but over 80% are the result of infection by a diverse set of uropathogenic E. coli strains, collectively referred to as UPEC. While a vaccine against UPEC would not be universally effective in the human population, it would represent a dramatic step towards changing how UTIs are prevented and treated. UPEC themselves are a heterogeneous set of strains with variable expression of virulence factors, such that a vaccine would still likely need to target multiple antigens to be broadly effective against UPEC alone. For these reasons, we sought to design a multivalent vaccine that would broadly target UPEC strains to prevent a large majority of urinary tract infections.

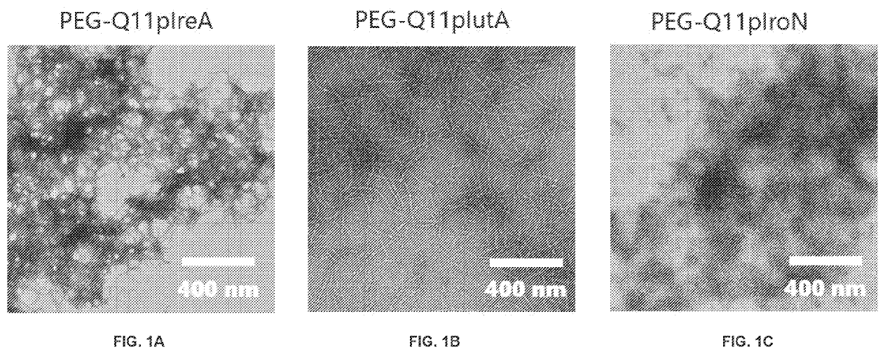

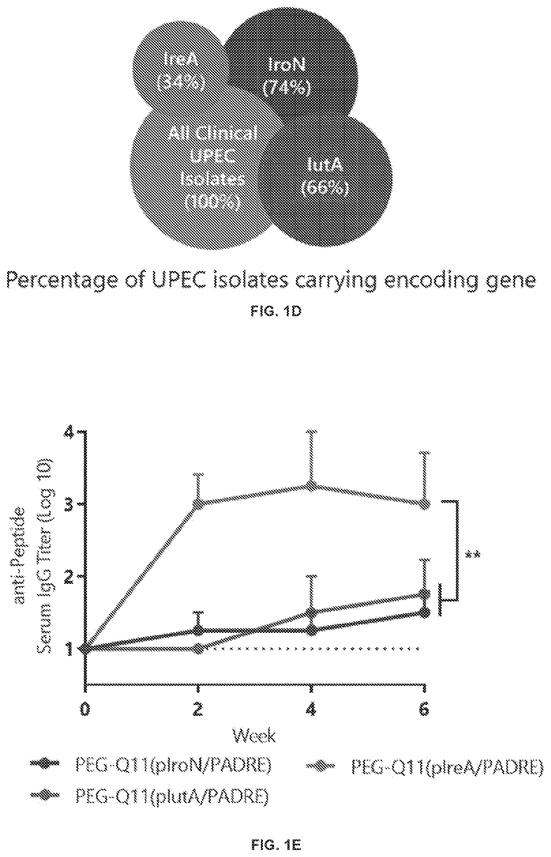

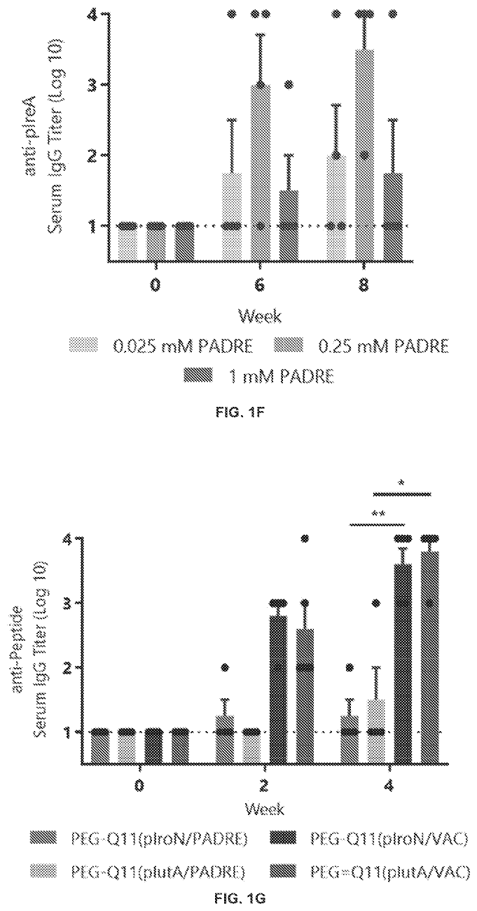

To prioritize safety and avoid adverse effects related to off-target immune responses against non-pathogenic E. coli or other commensals, we sought peptide epitopes that would be found near-exclusively on UPEC. A major set of UPEC virulence factors are iron receptor proteins, which allow UPEC to survive in the iron-poor urinary tract. These receptors are ideal antigenic targets because, in addition to being critical to the survival of UPEC, they are surface-expressed, allowing for possible antibody binding. We selected three peptide epitopes previously identified within UPEC receptor proteins, one each from the proteins IreA, lutA, and IroN. The genes encoding these proteins were found in 34%, 66%, and 74% of clinical UPEC isolates, respectively, suggesting that by targeting all three we could design a vaccine that would be broadly effective against human infection with UPEC (FIG. 1D). Notably, the peptide epitopes from lutA and IroN have previously shown poor immunogenicity in mice after mucosal immunization, failing to raise systemic or urinary antibody responses even when given with high doses of the strong but non-translatable cholera toxin adjuvant. We viewed these poorly immunogenic peptide epitopes as an opportunity to utilize our sublingual peptide immunization technology.

### Example 3

### Co-Assembly of T-Cell and B-Cell Epitopes in Sublingual Nanofiber Vaccines Elicits Antibody Responses Against UPEC Epitopes

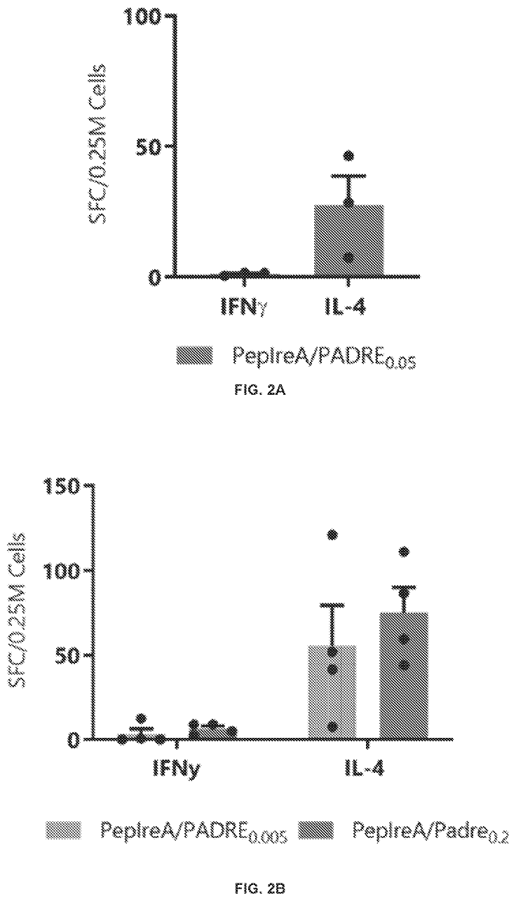

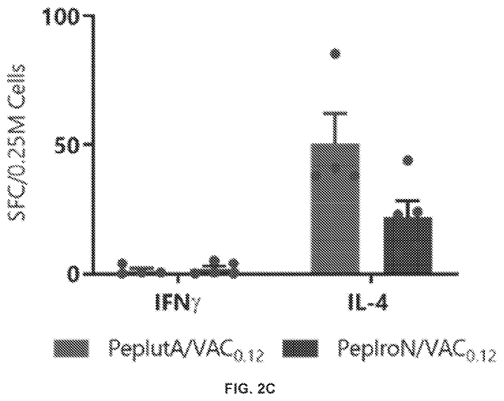

We sought to leverage our recently developed sublingual peptide vaccine platform to enhance the immunogenicity of these challenging peptide epitopes and develop a highly specific anti-UPEC vaccine. We designed self-assembling peptide-polymers with C-terminal peptide epitopes (referred to hereafter as plreA, plutA, and plroN to distinguish them from their parent proteins), N-terminal PEG chains to promote sublingual mucus transport, and central Q11 peptide self-assembly domains to drive self-assembly into nanofibers (sequences are in TABLE 1). Each of the constructs—PEG-Q11plreA, PEG-Q11plutA, and PEG-Q11plroN—assembled into supramolecular nanofibers, as evidenced by electron microscopy (FIGS. 1A-1C). We co-assembled nanofibers containing both the UPEC B-cell epitopes and the universal helper T-cell epitope PADRE (Del Guercio, M.-F., et al. Vaccine 1997, 15, 441-448, incorporated herein by reference). Sublingual immunization with 8 μL droplets of solutions containing PEG-Q11 (plreA/PADRE) fibers and the adjuvant CTB (the non-toxic B subunit of cholera toxin) led to robust plreA-specific serum antibody responses (FIG. 1E). IL-4⁺ T-cell responses against the PADRE epitope were also observed (FIGS. 2A-2C). Notably, the antibody responses were dependent on the ratio of B- to T-cell epitope within the nanofibers (FIG. 1F). Co-assembly of PADRE did not promote antibody responses against either plutA or plroN, leading us to use an alternate universal T-helper epitope from vaccinia virus (Mora-Solano, C., et al. Biomaterials 2017, 149, 1-11, incorporated herein by reference) (referred to here as VAC). Sublingual immunization with PEG-Q11 (plutA/VAC) and PEG-Q11 (plroN/VAC) elicited epitope-specific antibody responses (FIG. 1G). The efficacy of the selected VAC epitope over PADRE in this instance could be due to differences in affinity of binding between the epitopes and MHC-II molecules. The reported affinity of the VAC epitope for I-Ab (the haplotype of C57BL/6 mice) is 13.5 nM, compared with 94 nM for the PADRE epitope. These results highlighted the ability of our vaccine platform to raise antibody responses sublingually against poorly immunogenic peptide epitopes, and the modularity of the materials made it straightforward to assess different types and amounts of T-cell epitopes for advancement into the subsequent studies.

### Example 4

### Multivalent Nanofibers Raise Simultaneous Responses Against Selected UPEC B-Cell Epitopes in Serum and Urine

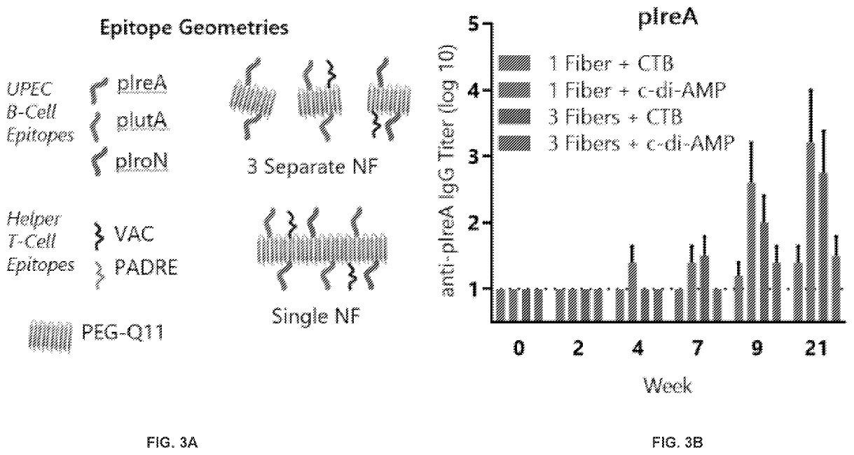

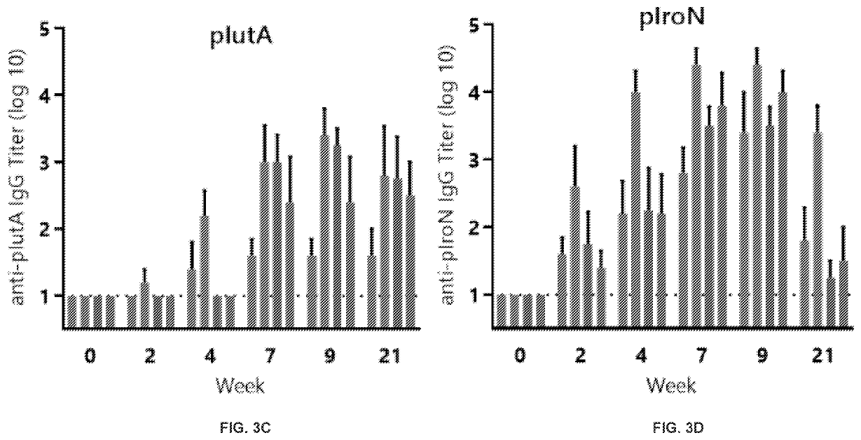

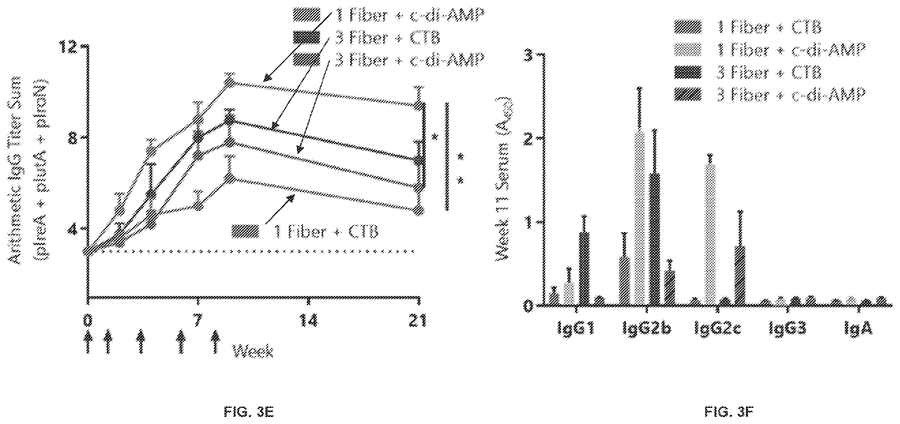

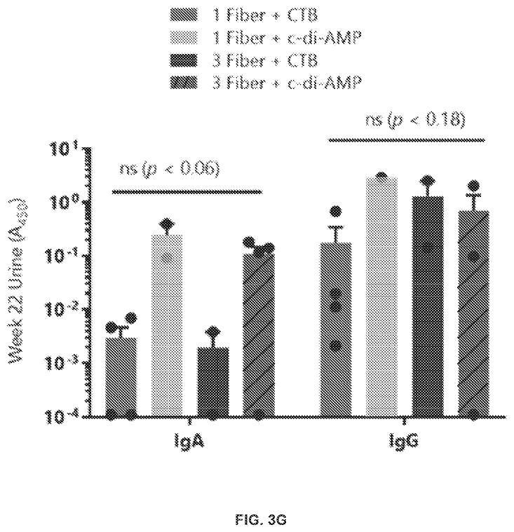

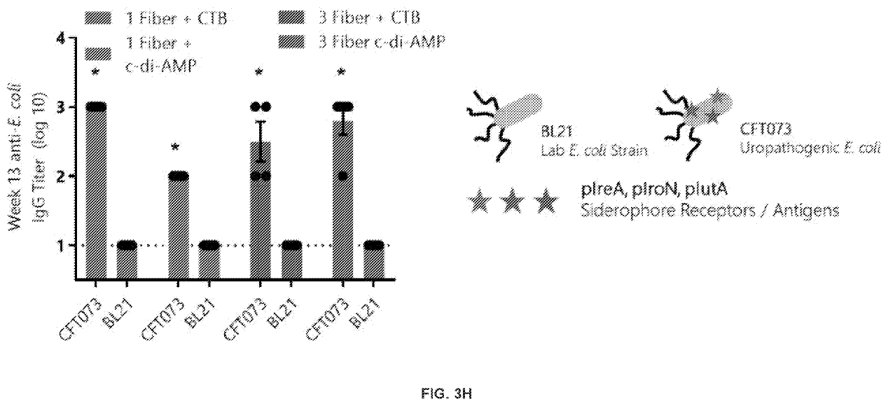

Having established that the selected B-cell epitopes were immunogenic in peptide nanofibers, we next sought to design a multi-epitope vaccine to elicit simultaneous antibody responses against all three epitopes, plreA, plutA, and plroN. Again, we took advantage of the modularity of self-assembled peptide vaccines, which enabled a straightforward investigation of how the epitope arrangement within the nanofibers affected antibody responses in the context of different adjuvants. We immunized one set of mice with self-assemblies where all peptides were co-assembled into homogenous nanofibers bearing all three B-cell epitopes and one T-cell epitope, termed PEG-Q11 (plreA/plutA/plroN/VAC) (FIG. 3A). For another set, we synthesized three types of nanofibers, each bearing only one of the B-cell epitopes along with T-cell epitopes, and we immunized mice with mixtures of these 3 nanofibers, termed (PEG-Q11 (plreA/PADRE)+PEG-Q11 (plutA/VAC)+PEG-Q11 (plroN/VAC)). We further compared these two formulations when adjuvanted with either CTB adjuvant or the STING agonist cyclic-di-AMP. Each of the four formulations elicited epitope-specific antibody responses against each of the three UPEC B-cell epitopes (FIGS. 3B-3D). As a measure of the overall strength of the response, we calculated an arithmetic sum of the log 10 IgG endpoint titers against the three epitopes. By this combined measure, the single nanofiber bearing all three B-cell epitopes and adjuvanted with cyclic-di-AMP elicited the strongest responses (FIG. 3E). The IgG subclasses of these responses were predominantly IgG2b and IgG2c, subclasses that are in many cases the most potent for anti-bacterial immunity (FIG. 3F). The simplicity of the single nanofiber formulation and favorable translational profile of cyclic-di-AMP, along with its effectiveness at raising high-titer responses, led us to select this formulation for future studies.

One advantage of using sublingual immunization as the route for a UTI vaccine is its demonstrated ability to raise immune responses in the urinary tract. To characterize the ability of our vaccines to raise antigen-specific IgG and IgA within the urinary tract, we assayed urine from immunized mice (FIG. 3G). Individual groups were not statistically different from each other, though comparisons of IgA responses against different formulations approached significance, so to test the impact of adjuvant and nanofiber composition separately, we combined groups and ran t-tests on urinary antibody levels, using Holm-Šídak correction for multiple comparisons. Nanofiber composition was not significant for either IgG (p=0.95) or IgA (p=0.95) when CTB and cyclic-di-AMP adjuvanted groups were combined. In contrast, we found that cyclic-di-AMP produced significantly higher epitope-specific IgA levels in the urine than CTB when one nanofiber and three nanofiber groups were combined (p=0.038), while IgG levels did not differ significantly (p=0.38). These results indicated that nanofiber sublingual vaccines could raise responses not only systemically, but also within the urinary tract.

### Example 5

### Vaccine-Induced Antibody Responses are Specific for Uropathogenic E. coli

One motivation for utilizing peptide epitopes is that despite their generally poor immunogenicity, they are highly specific and in principle can be selected to target pathogenic bacterial strains while sparing non-pathogenic strains. To test the specificity of the antibody response raised by our vaccine, we compared their level of binding to both a uropathogenic strain of E. coli (CFT073) and a non-pathogenic lab strain of E. coli (BL21). Serum antibodies from all four immunization groups bound to CFT073 but had no detectable levels of anti-BL21 antibodies (FIG. 3H). The CFT073 culture conditions used for this ELISA were expected to induce expression of plutA, but not plreA or plroN. These results still highlighted that the induced antibodies do not bind non-pathogenic E. coli, and that antibodies raised by peptide nanofibers are able to bind to CFT073. Along with the demonstrated ability to raise multivalent responses and to elicit urinary antibodies, this specificity is a unique advantage.

### Example 6

### STING and TLR9 Agonists Enhance Urinary Antibody Levels after Sublingual Nanofiber Immunization

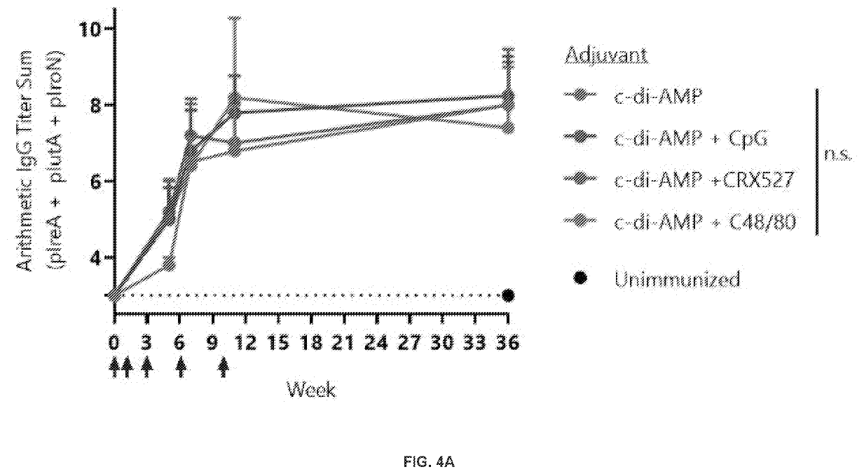

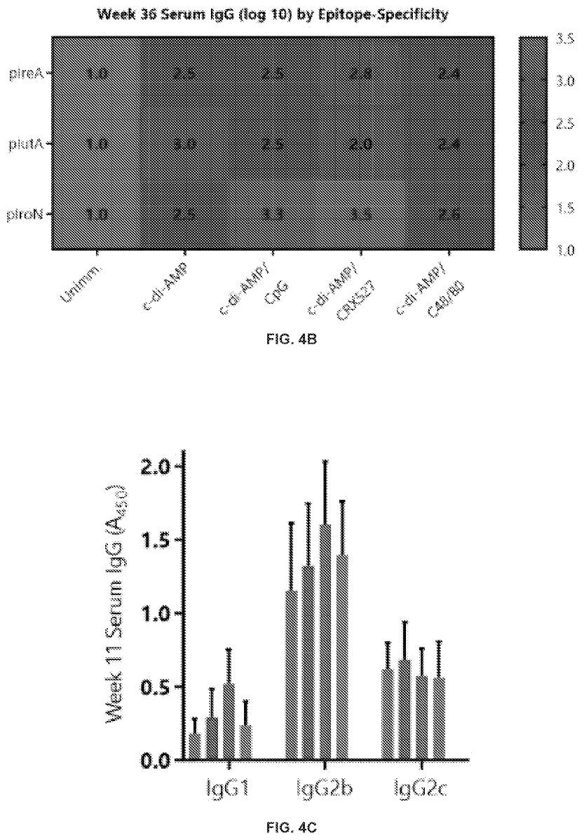

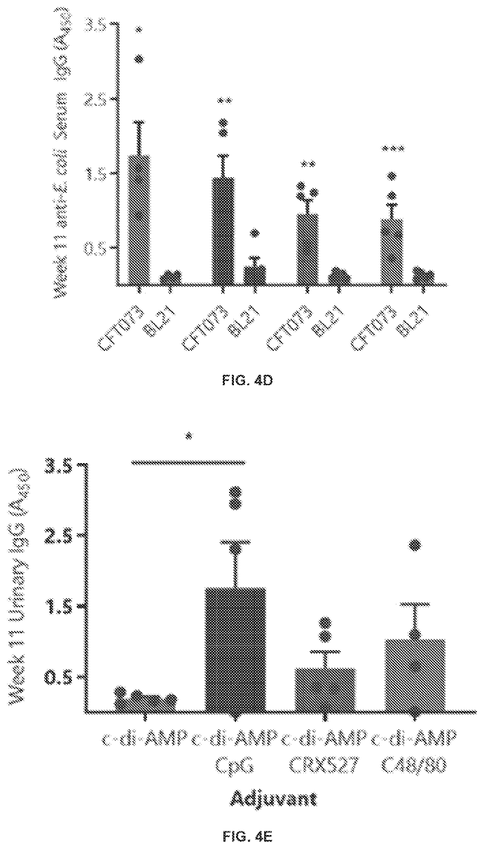

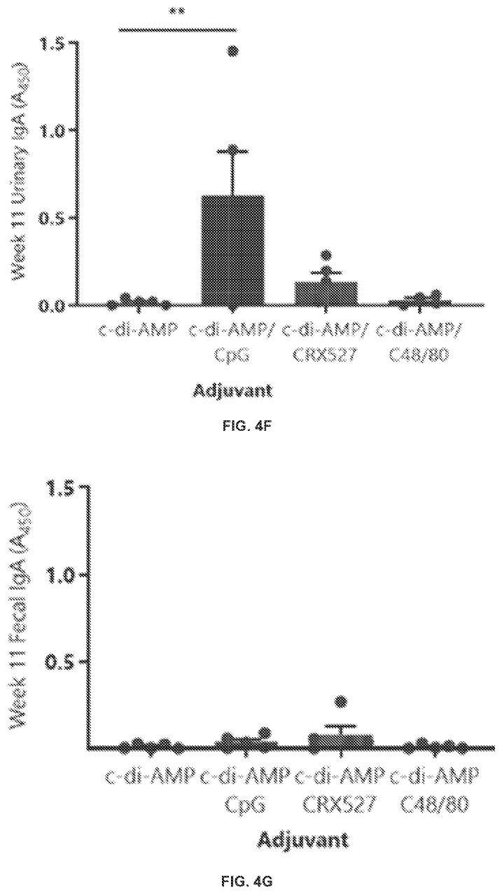

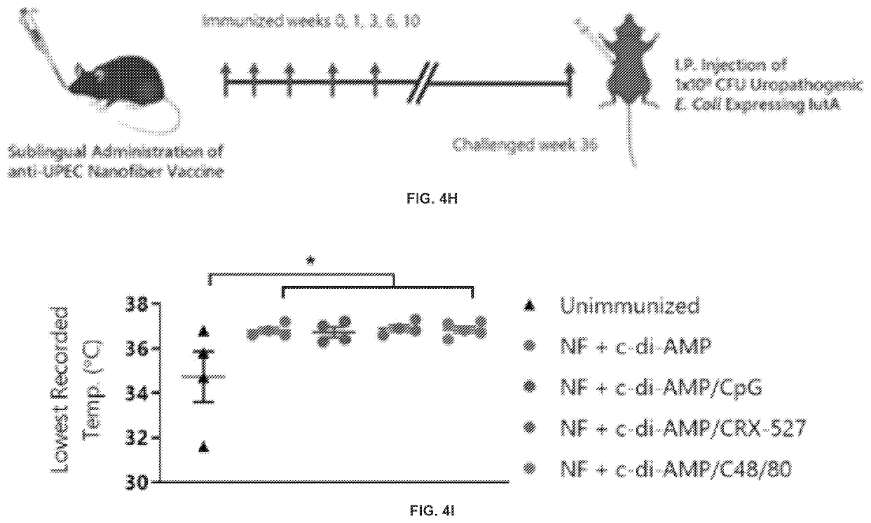

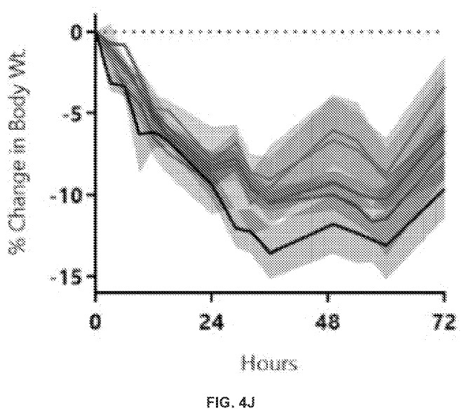

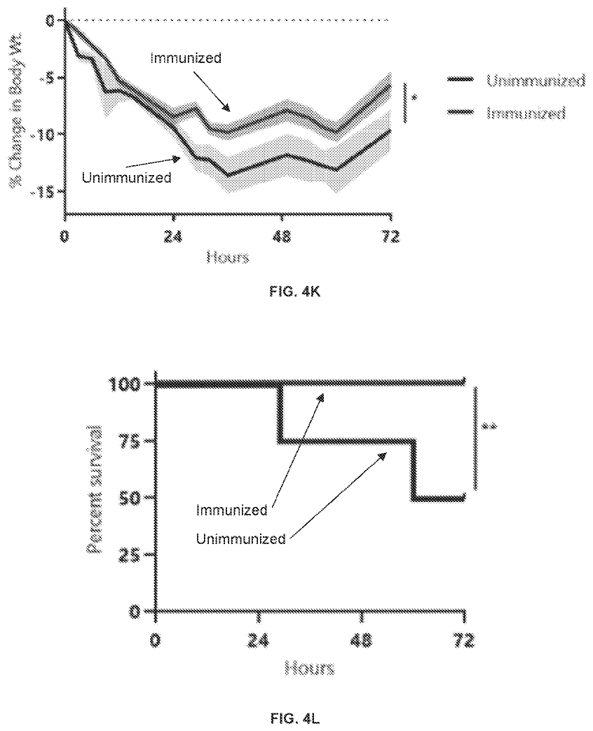

Based on our initial vaccine studies, we proceeded with a formulation of a single co-assembled nanofiber and cyclic-di-AMP adjuvant. To enhance the urinary antibody responses elicited by our initial formulations, we examined the effects of combining cyclic-di-AMP with additional adjuvants that act through different and potentially complementary pathways, such as those induced by toll-like receptors (TLRs). Combinatory adjuvant approaches are well-studied in the nanomaterials community and have recently been applied in the context of UPEC vaccines. We sublingually immunized mice with PEG-Q11 (plreA/plutA/plroN/VAC) nanofibers and cyclic-di-AMP, plus either CpG (a TLR9 agonist), CRX527 (a TLR4 agonist), or C48/80 (a mast cell stimulator). The addition of these adjuvants did not have a significant effect on serum antibody responses, as all groups raised multivalent responses against all three B-cell epitopes that were primarily of the IgG2b and IgG2c subclasses (FIGS. 4A-4C). In contrast to the serum responses, however, the adjuvant combination strategy led to a significant increase in urinary antibodies. Specifically, the combination of cyclic-di-AMP and CpG led to significantly greater levels of epitope-specific IgA in the urine and urinary IgG levels that trended higher (FIG. 4D).

### Example 7

### Sublingual Nanofiber Vaccine Raises No Detectable Gut IgA Responses

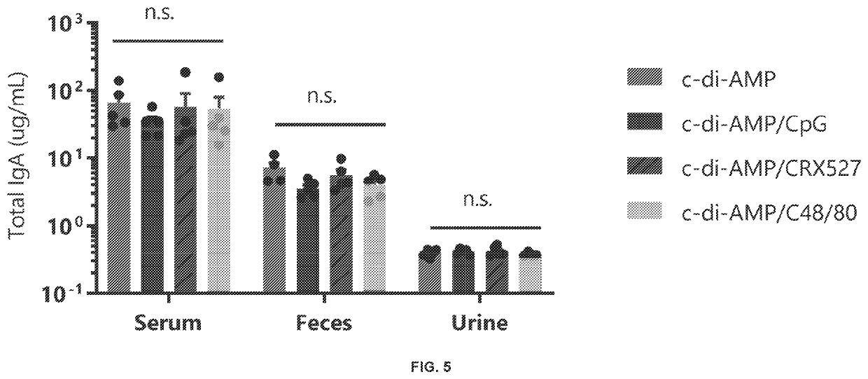

An advantage of UPEC-specific vaccine strategies is that they avoid potential immune responses against commensal E. coli or other commensals. A further safeguard against such effects is a vaccine that raises antibody responses in the desired systemic and urinary compartments, but not in the microbe-rich gut. Sublingual vaccines have been shown in some cases to raise immune responses in the gastrointestinal tract. Swallowing of vaccine material could contribute to gut responses; notably, we used a conservative volume of 8 μL for our sublingual immunizations, well below the amount at which inadvertent swallowing would be expected. We tested whether the sublingual nanofiber vaccines were eliciting gut antibodies along with those observed in the blood and urine. In contrast to the observable levels of IgA, we did not detect any epitope-specific IgA in fecal samples of immunized mice (FIG. 4G). To confirm that our methods could detect fecal IgA, we examined the total fecal IgA and found it to be similar to published levels for C57BL/6 mice (Fransen, F., et al. Immunity 2015, 43, 527-540, incorporated herein by reference) (FIG. 5).

### Example 8

### Sublingual Vaccine Protects Against UPEC-Mediated Sepsis with Expression of a Single Antigen Target

To test the clinical relevance of the antibodies raised by our sublingual vaccine, we challenged mice with CFT073, a uropathogenic E. coli strain isolated from a pyelonephritis patient (Mobley, H., et al. Infection and immunity 1990, 58, 1281-1289, incorporated herein by reference). To directly assay the effectiveness of raised antibodies against the human pathogen, we performed intraperitoneal challenge of the previously immunized mice discussed above (FIG. 4H). This challenge also provides a model of UPEC-mediated sepsis; notably, urosepsis accounts for a quarter of all sepsis cases. The motivation for a multivalent vaccine strategy was to generate an immune response that is protective against UPEC with varying virulence factor expression profiles. To test whether expression of a single antigenic target on the infecting pathogen was sufficient for the nanofiber vaccine to afford protection, we cultured CFT073 under conditions in which the lutA protein, but neither the IreA nor IroN proteins, is expressed (Hagen, Iron Acquisition by Uropathogenic Escherichia coli: ChuA and Hma Heme Receptors as Virulence Determinants and Vaccine Targets, 2009, incorporated herein by reference).

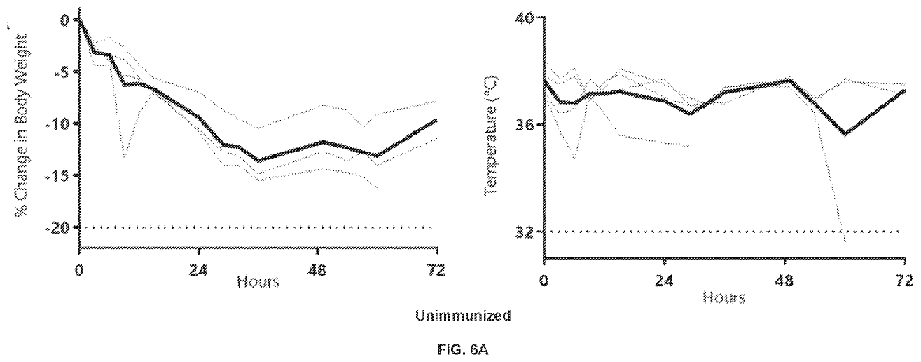

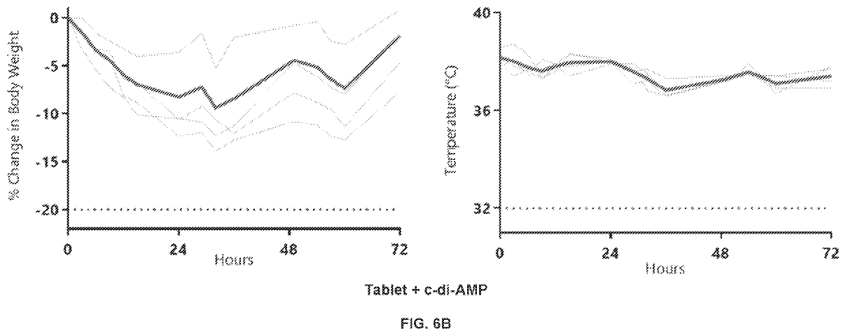

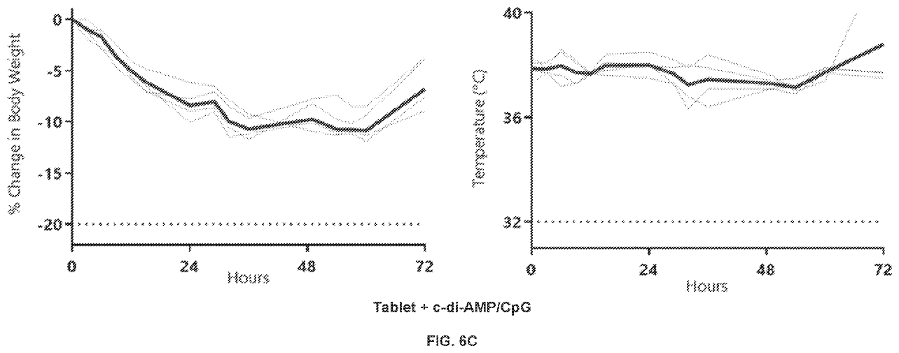

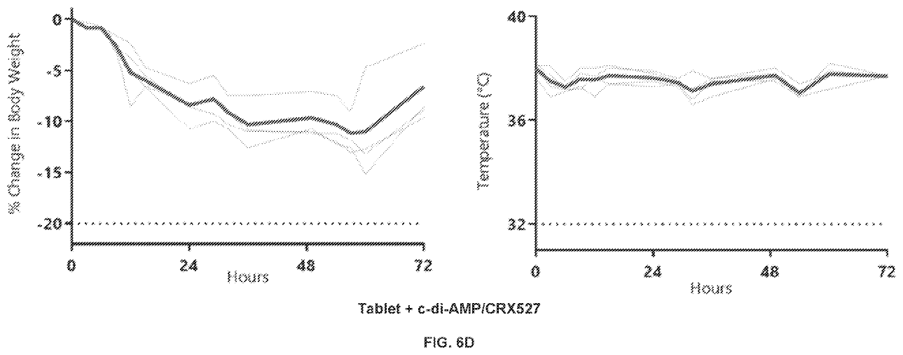

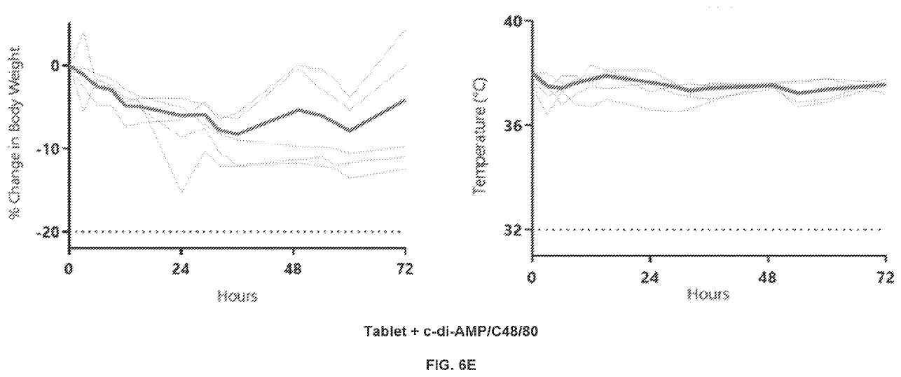

After challenging mice with CFT073, we monitored body temperature and weight loss for 72 h to see if vaccinated groups would ameliorate these expected clinical symptoms of infection (Olfert et al. Ilar Journal 2000, 41, 99-104, incorporated herein by reference). Unimmunized control mice exhibited significant loss of body temperature after challenge, compared with vaccinated mice (FIG. 4I, FIGS. 6A-6E). Body weight showed more variance between different immunization groups (which differed by adjuvant), but all immunized groups lost less weight compared with unimmunized mice throughout the study (FIG. 4J, FIGS. 6A-6E). To compare the overall effect of immunization in protecting against CFT073 challenge, we combined the results from the four groups of immunized mice. Collectively, the immunized mice showed significantly less change in body weight than unimmunized mice (FIG. 4K). Further, half of the unimmunized mice died as a result of the challenge, while all vaccinated mice survived (FIG. 4L). These results together indicated that the sublingual nanofiber vaccine provided protection against challenge with a human-infecting UPEC strain expressing a single targeted antigen.

### Example 9

### An Accessible Tablet Vaccine Formulation Raises Protective Immune Responses Against UPEC Epitopes in Mice

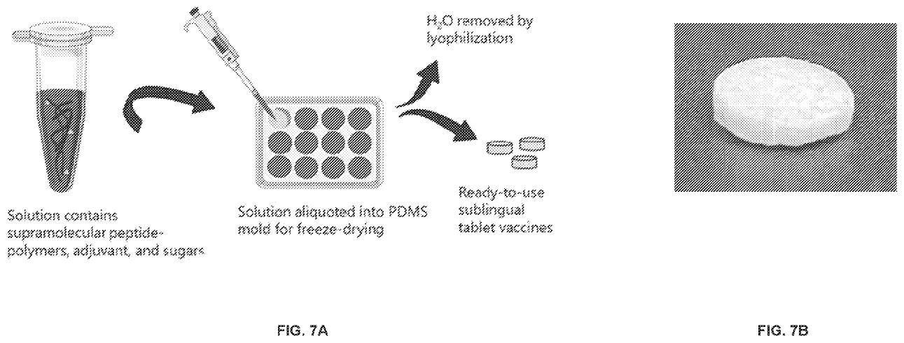

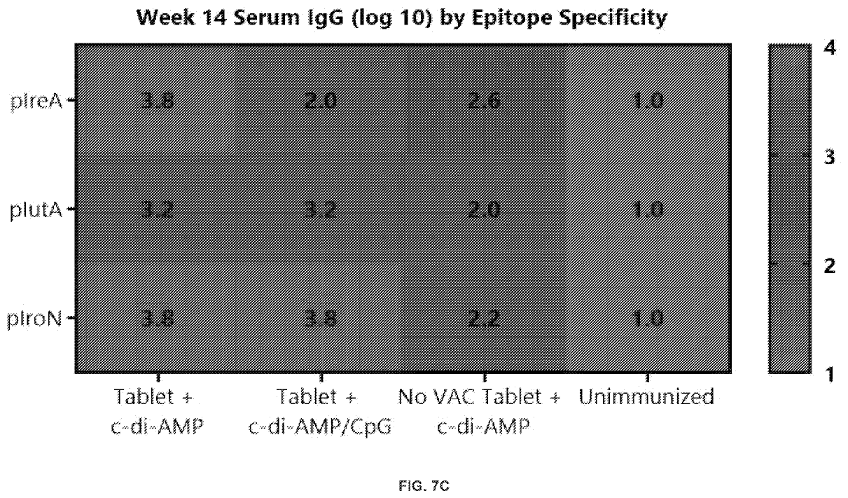

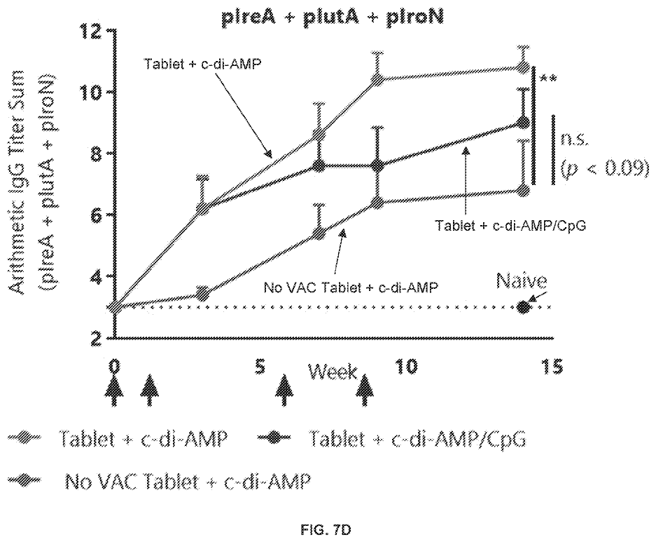

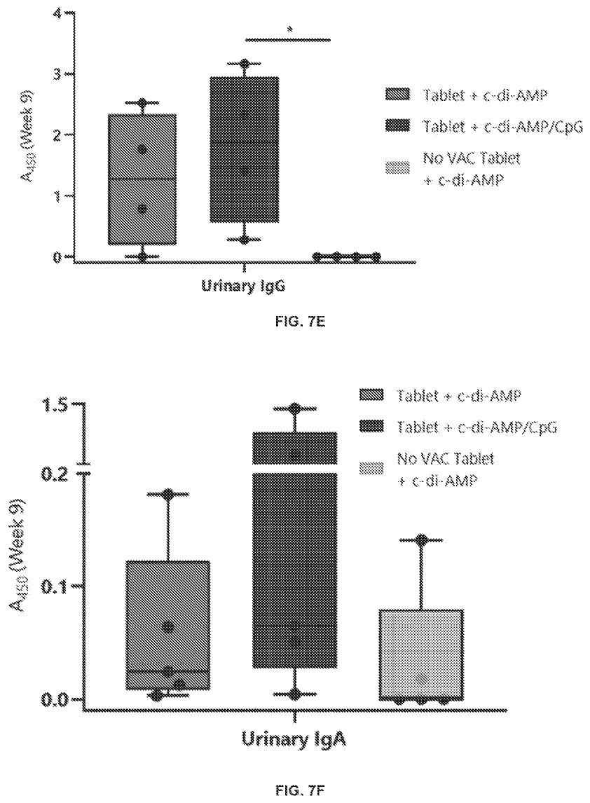

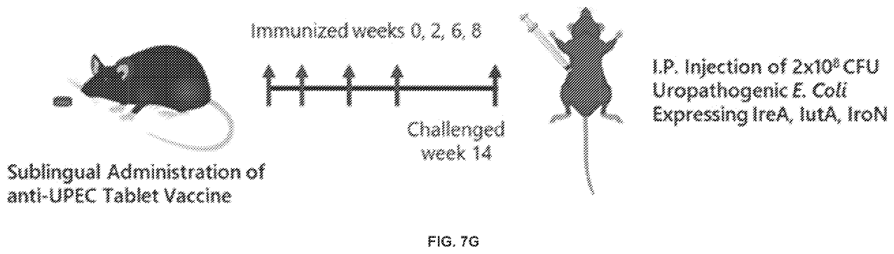

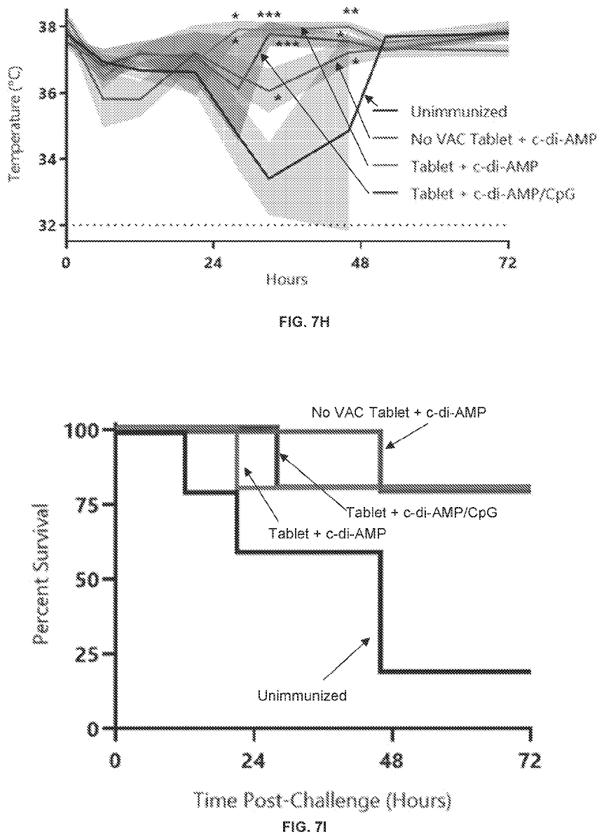

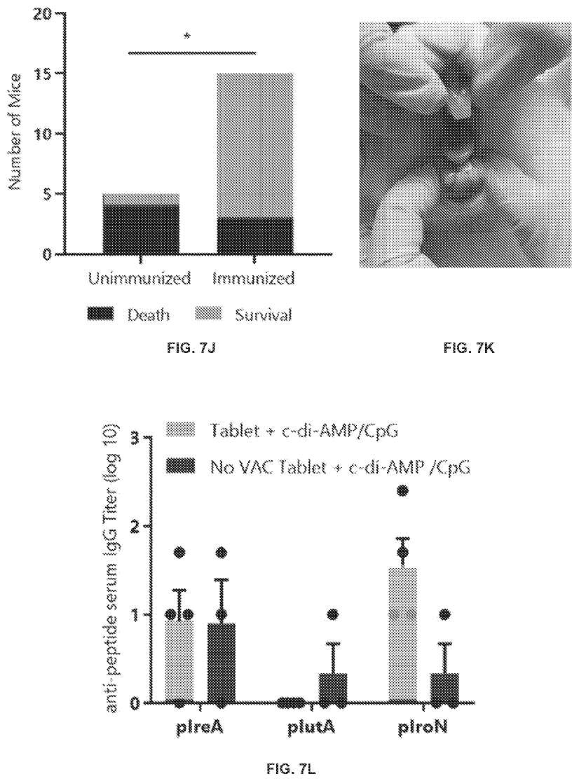

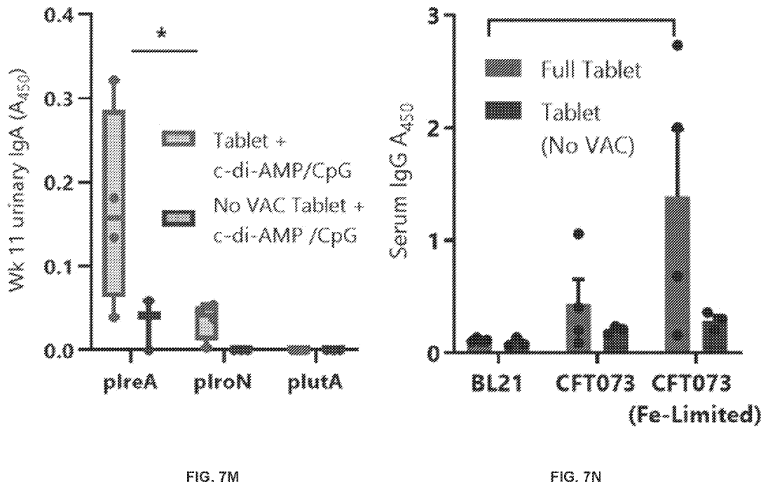

Attitudes and perceptions regarding UTIs and resource limitations put constraints on the potential distribution of an anti-UPEC vaccine, particularly outside of wealthy nations. To achieve its most significant impact, a UTI vaccine would likely need to be cost-effective, suggesting the need for heat-stability (to obviate expensive cold chain storage) and simple administration procedures requiring minimal training. We recently developed a procedure for producing tablet vaccines based on self-assembling peptide-polymer nanofibers, which we termed SIMPL (Supramolecular Immunization with Peptides SubLingually) (Kelly, et al. Advanced Healthcare Materials 2021, 10, 2001614, incorporated herein by reference). SIMPL tablets are produced by lyophilization of solutions containing supramolecular nanofibers, sugar excipients, and adjuvant (FIG. 7A).

To test the effectiveness of a tablet formulation of an anti-UPEC nanofiber vaccine, we formed tablets containing PEG-Q11 (plreA/plutA/plroN/VAC) (FIG. 7B). We included either cyclic-di-AMP adjuvant alone or both cyclic-di-AMP and CpG, based on our finding that this adjuvant combination could enhance urinary antibody responses (FIGS. 4A-4L). As a control, we also produced tablets containing PEG-Q11 (plreA/plutA/plroN) fibers, which lack the helper T-cell epitope VAC (VACQ11 was replaced with Q11 in the formulation to keep total peptide concentration constant). Sublingual immunization with vaccine tablets performed similarly to the previously used droplet vaccines, raising multivalent responses against all three UPEC B-cell epitopes (FIG. 7C). Among tablets with only the cyclic-di-AMP adjuvant, tablets without the VAC epitope raised significantly lower serum antibody responses than those containing the T-cell epitope (FIG. 7D).

Interestingly, tablets without VAC did still raise detectable antibody responses against each of the epitopes, indicating that one of plreA, plutA, or plroN may contain a T-cell epitope within its sequence. Putative epitopes within the plreA sequence have been reported for BALB/c mice (H-2-IAd haplotype), though to our knowledge, T-cell epitopes within these peptides have not been reported for C57BL/6 mice. An in silico prediction we performed using the Consensus method (Wang, et al. BMC Bioinformatics 2010, 11, 1-12; Wang, et al. PLoS Computational Biology 2008, 4, e1000048; each incorporated herein by reference) in the Immune Epitope Database and Analysis Resource (IEDB) did show potentially strong binding sequences to H-2-IAb within the plreA sequence, which may in part account for the responses seen here.

The highest level of urinary IgG was observed in mice receiving tablets with the VAC epitope and both cyclic-di-AMP and CpG adjuvants, again indicating that adjuvant combinations may be particularly important for stimulation of mucosal responses after sublingual nanofiber immunization (FIG. 7E). There was no detectable urinary IgG in mice that received tablets without the VAC epitope. Urinary IgA levels were not significantly different between groups but trended highest for the adjuvant combination group (FIG. 7F).

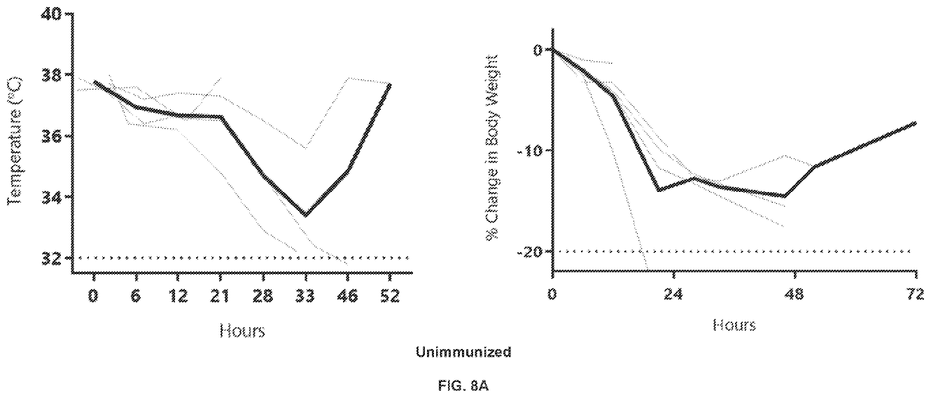

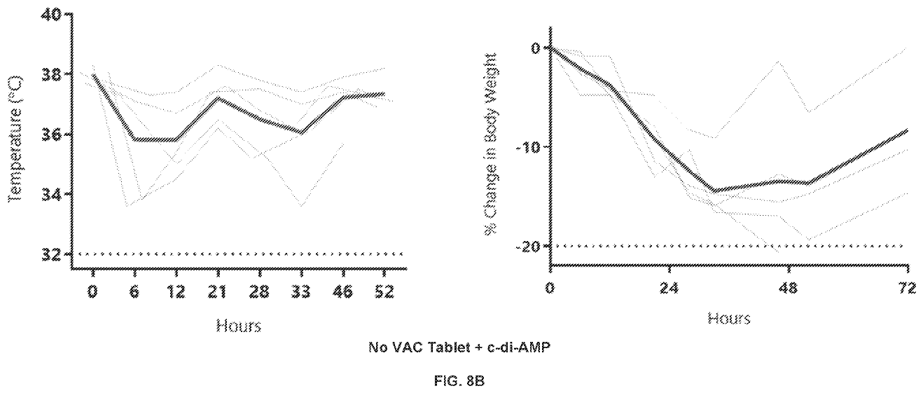

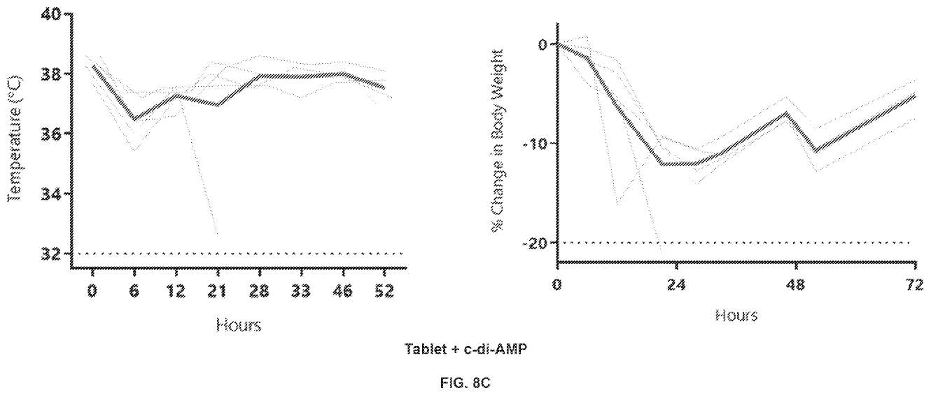

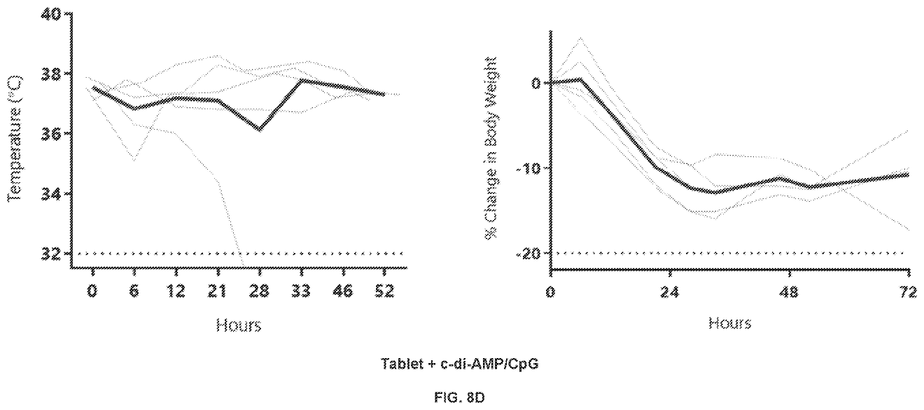

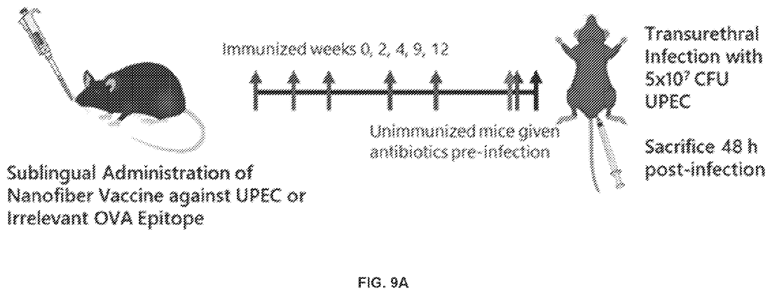

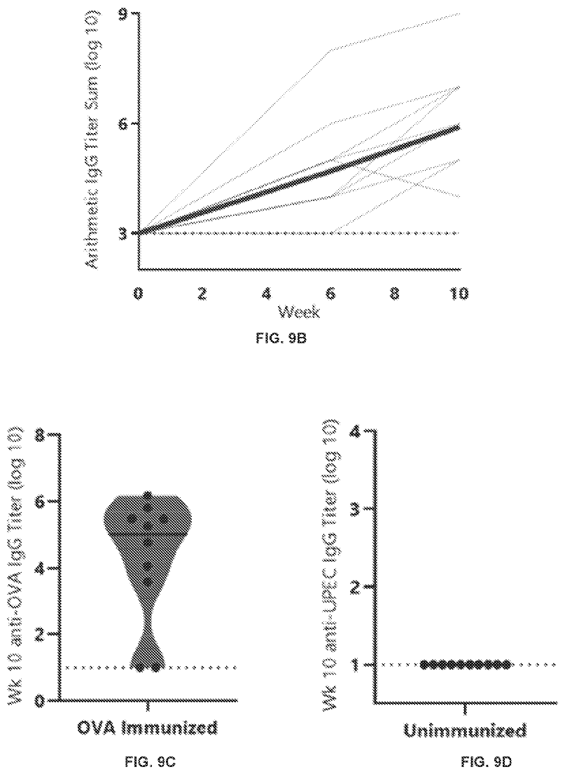

We next tested the protective ability of immune responses elicited by anti-UPEC SIMPL tablets. We challenged mice with a higher-lethality dose of 2×10⁸ CFU of CFT073, culturing these bacteria in iron-limited conditions that promote expression of IreA, lutA, and IroN proteins (Hagan, Iron Acquisition by Uropathogenic Escherichia coli: ChuA and Hma Heme Receptors as Virulence Determinants and Vaccine Targets, 2009, incorporated herein by reference) (FIG. 7G). Nanofiber tablet vaccines were highly protective even against this increased dose, significantly ameliorating temperature drops and increasing survival (FIGS. 7H-7M, FIGS. 8A-8D). Immunization with any of the three tablet formulations led to 80% survival, compared with just 20% in unimmunized control groups.

### Example 10

### Anti-UPEC Sublingual Tablet Vaccine Raises Epitope-Specific Serum and Urinary Antibodies in Rabbits

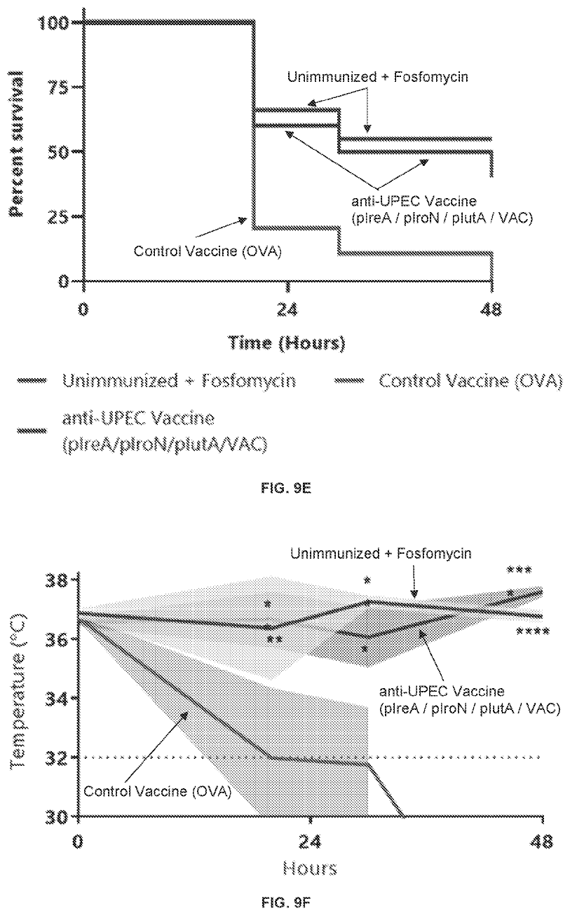

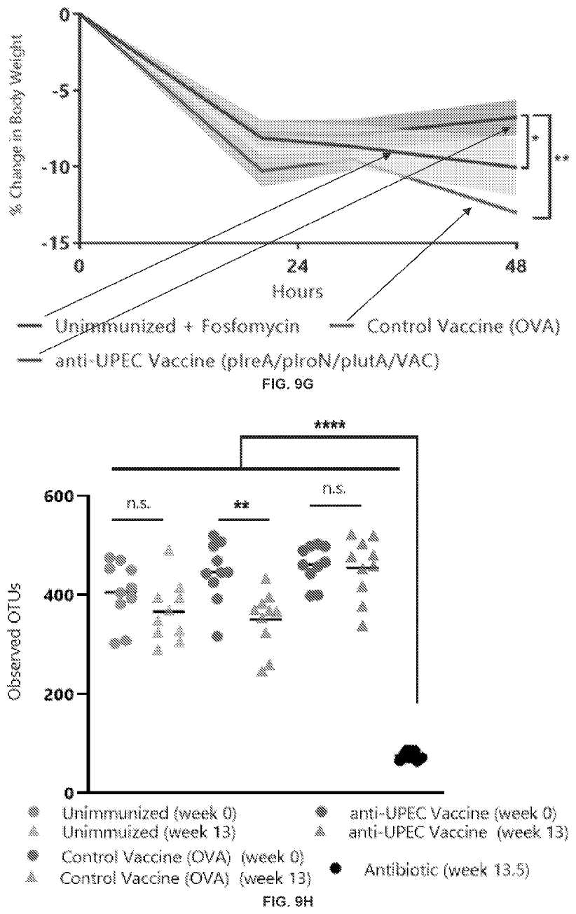

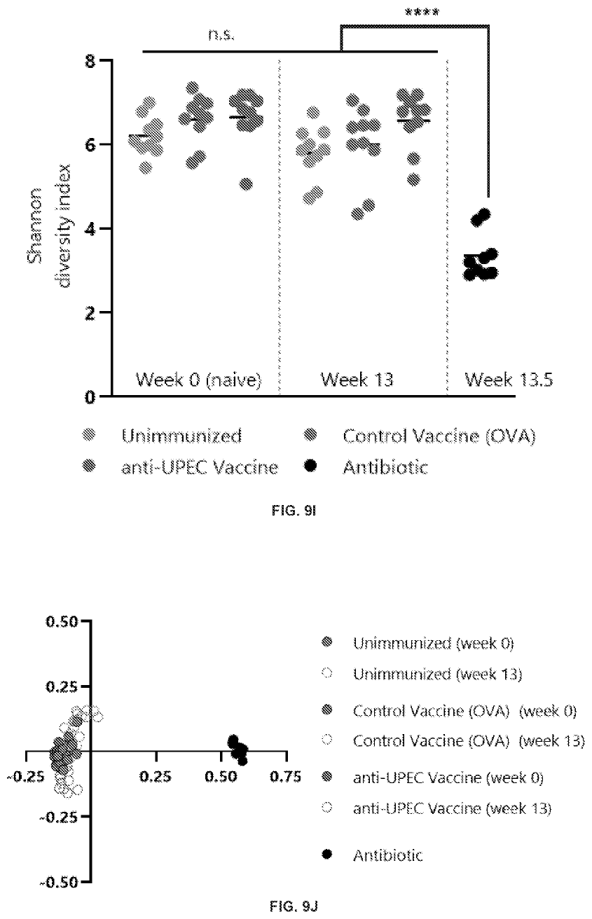

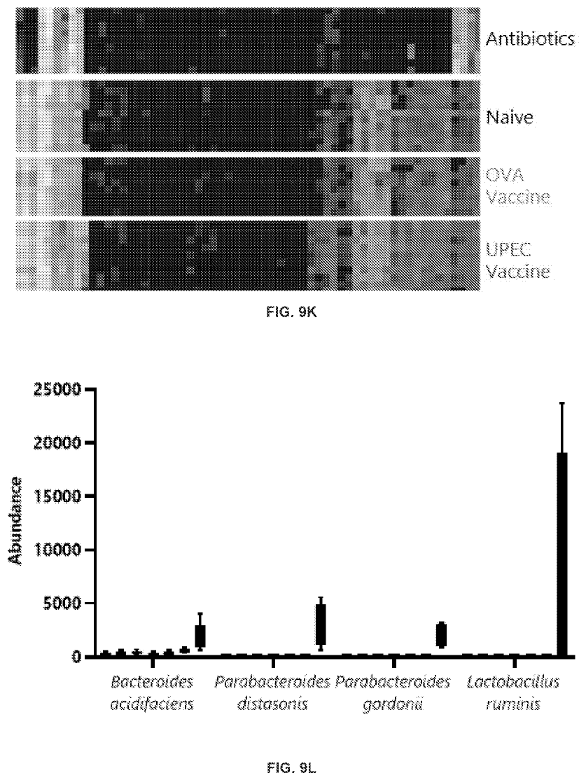

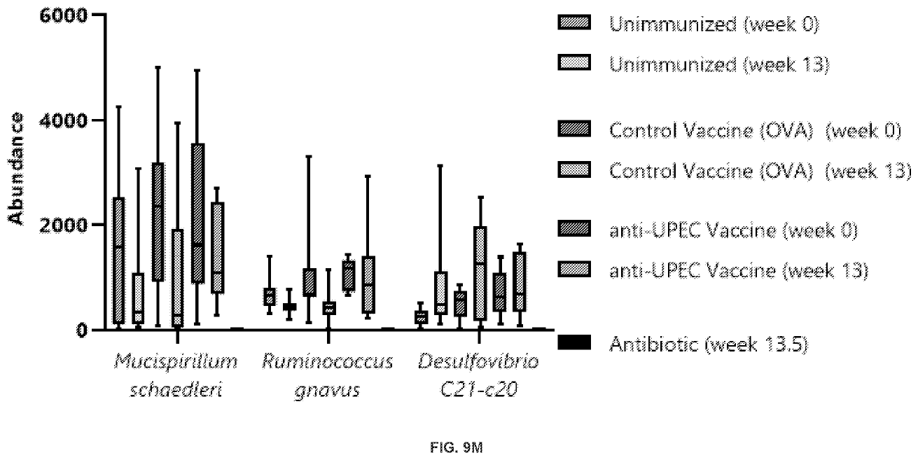

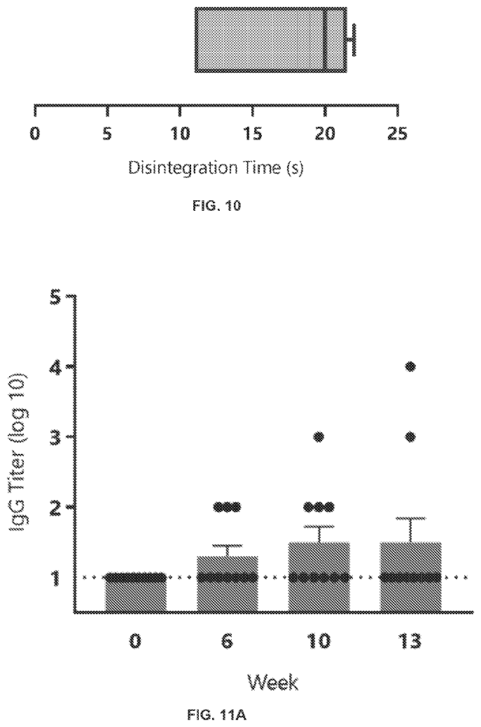

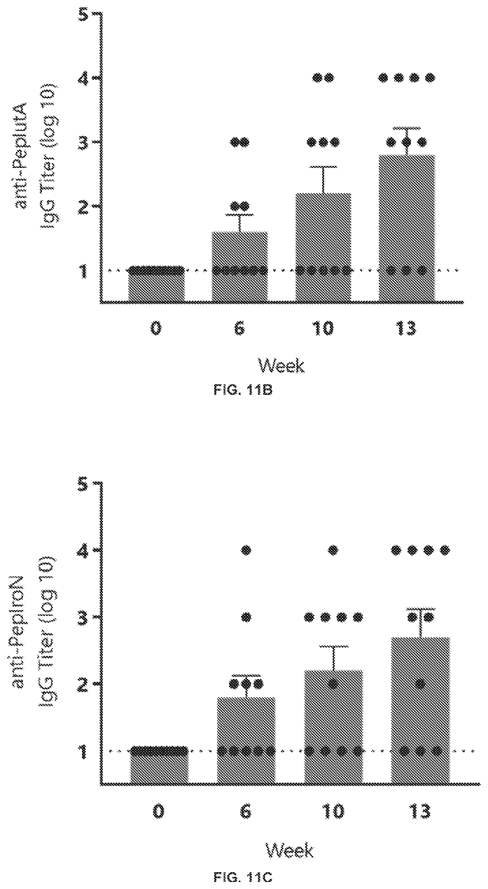

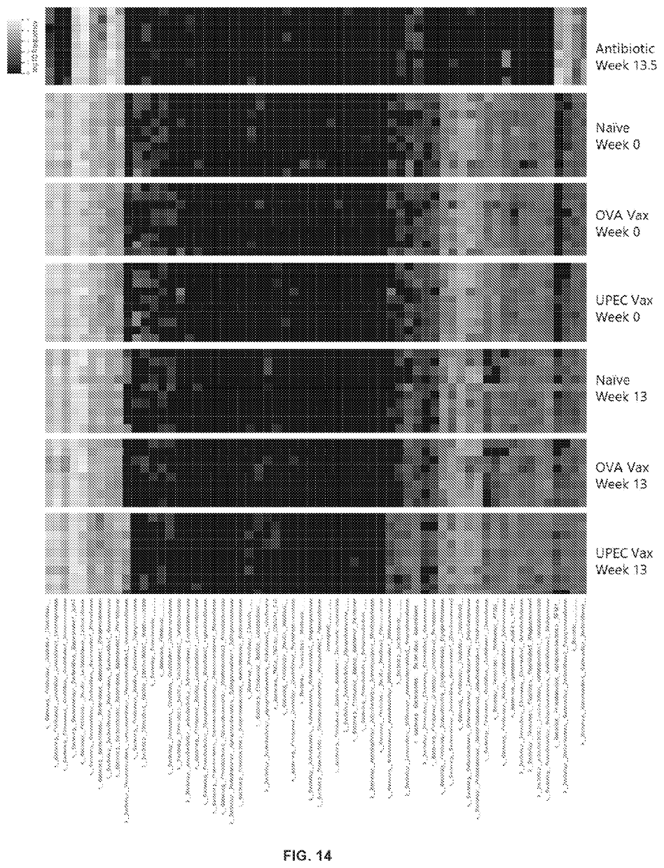

To further demonstrate the translational potential of anti-UPEC nanofiber vaccines, we sought to use a more representative higher animal model. Compared with mice, rabbits have an oral cavity that is more similar to that of humans. Importantly, the sublingual epithelium is non-keratinized in rabbits and humans (but keratinized in mice), and it contains greater numbers of cell layers in both rabbits and humans than in mice. These differences make rabbits a more desirable model for assessing the potential clinical effectiveness of a sublingually delivered vaccine. Immunization of rabbits with SIMPL tablets containing PEG-Q11 (plreA/plutA/plroN/VAC) or PEG-Q11 (plreA/plutA/plroN; no VAC) nanofibers and both cyclic-di-AMP and CpG adjuvants elicited epitope-specific antibody responses (FIG. 9K, FIG. 9L). The full heatmap is shown in FIG. 14. Notably, the tablet immunizations were also capable of eliciting epitope-specific IgA responses in urine (FIG. 9I), and the antibodies bound most strongly to CFT073 cultured under iron-limiting uropathogenic conditions (FIG. 7N). Finally, to observe the dissolution behavior of the tablets in a simulated clinical setting, we simulated the human oral cavity using 1.0 ml of pooled human saliva heated to body temperature (37° C.) (FIG. 10). Tablets rapidly dissolved under these conditions, with a median time of 20 seconds. These results indicated the potential feasibility of progressing sublingual tablet vaccines towards clinical translation.

### Example 11

### Sublingual Nanofiber Vaccine is as Effective at Preventing UTI as High-Dose Oral Antibiotics

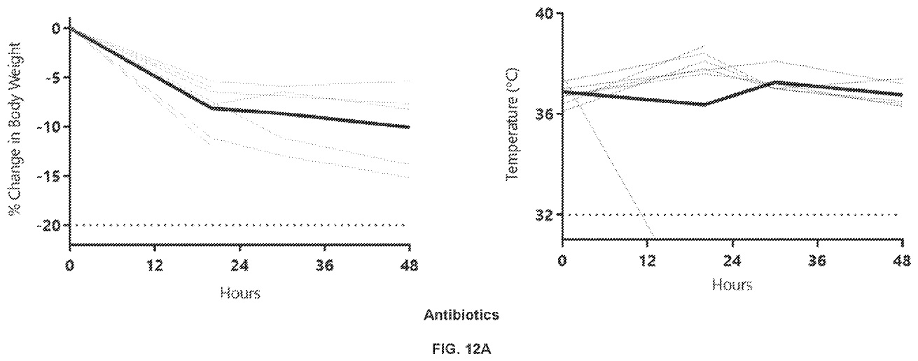

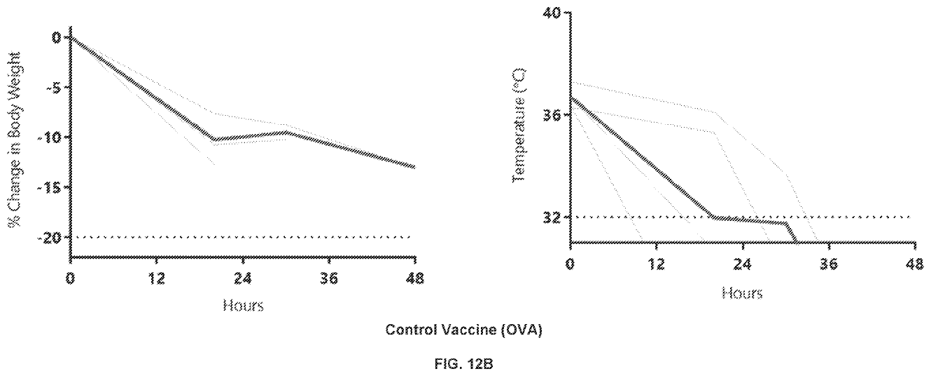

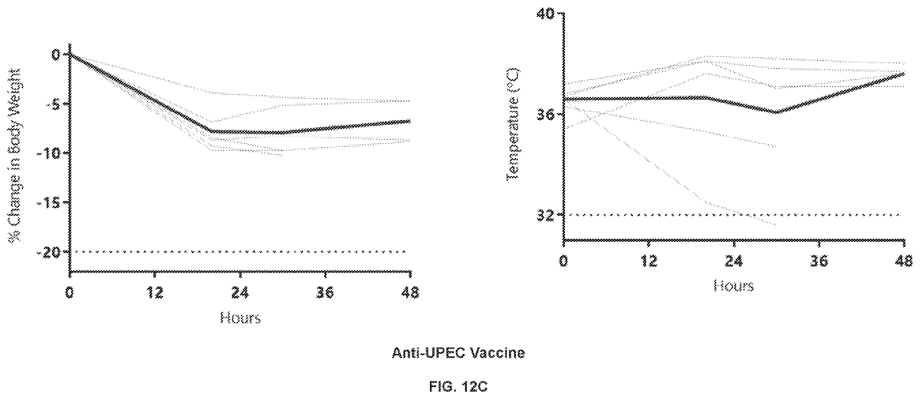

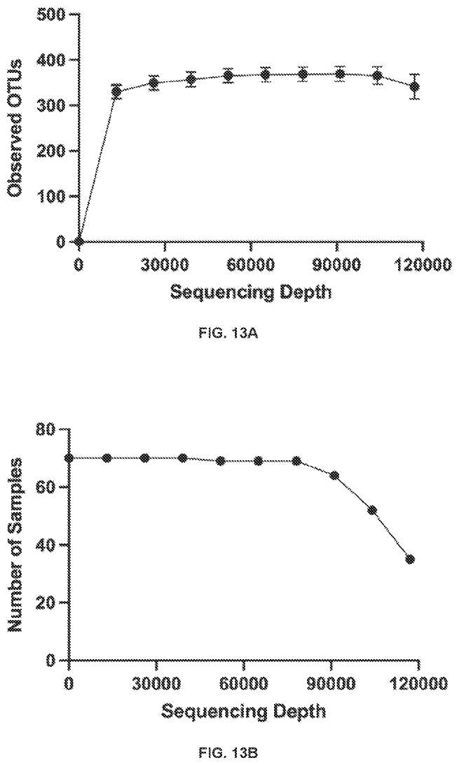

To place the efficacy of the anti-UPEC nanofiber vaccine in context, we compared it with antibiotics, the current gold-standard of treatment for UTIs. We immunized mice with cyclic-di-AMP and CpG adjuvants and either PEG-Q11 (PlreA/PlutA/PlroN/VAC) nanofibers or, as a control, PEG-Q11OVA nanofibers. OVA₃₂₃₋₃₃₉ is a model epitope that we have previously employed in Q11-based nanofibers for raising sublingual B- and T-cell responses (Kelly, S. H., et al. Biomaterials, 2020, 119903; Kelly, S. H., et al. Advanced healthcare materials 2021, 10, 2001614; each incorporated herein by reference) (FIG. 9A). As expected, mice given the anti-UPEC vaccine raised antibody responses against the plreA, plutA, and plroN epitopes, and OVA-immunized mice raised antibody responses against the OVA₃₂₃₋₃₃₉ epitope (FIGS. 9B-9C, FIGS. 11A-11C). We challenged immunized mice transurethrally with 5×10⁷ CFU CFT073 to model the route of urinary tract infection, and compared the results with unimmunized mice given repeated, high, daily doses of the oral antibiotic Fosfomycin (FIG. 9A). While no mice given the control OVA vaccine survived to 48 hours post-challenge, the vaccine performed similarly to the antibiotic treatment group (FIGS. 9D-9E). Notably, both the Fosfomycin-treated and anti-UPEC-immunized groups maintained significantly higher temperatures than mice given the control vaccine (FIG. 9F, FIGS. 13A-13B). Strikingly, mice given the nanofiber vaccine had significantly less body weight loss than mice treated with Fosfomycin (FIG. 9G, FIGS. 13A-13B). Weight and temperature curves for individual mice are shown in FIG. 12. The lack of efficacy of the control vaccine against OVA demonstrated that the effect of the anti-UPEC nanofiber vaccine was attributable to its antigen-specificity for CFT073, and not non-specific effects due to adjuvants or the nanofibers themselves.

### Example 12

### Anti-UPEC Vaccine is Minimally Disruptive of the Microbiome

While antibiotics are highly effective at treating UTIs, there are concerns about their long-term prophylactic usage due to effects on the patient's microbiome. We tested whether our vaccine strategy would allow for long-term protective responses that were minimally disruptive of the microbiome due to their specificity for uropathogenic E. coli. We collected feces from unimmunized, anti-UPEC immunized, or anti-OVA immunized mice prior to (week 0) and after (week 13) treatment. We also collected feces from the unimmunized group after 3 daily doses of Fosfomycin, prior to transurethral challenge (week 13.5).

The difference between the microbiome composition of antibiotic-treated mice and all other groups was stark. The OTU richness of the microbiome was drastically reduced after antibiotic treatment, while immunization with the anti-UPEC nanofiber vaccine had no effect (FIG. 9H). Further, while the Shannon diversity index (a measure of α-diversity) was statistically identical between all non-antibiotic treated groups, the antibiotic group was highly significantly different from these groups (FIG. 9I). To highlight this, we used multidimensional scaling to plot the similarity of each mouse (FIG. 9J). The antibiotic mice formed a distinct cluster apart from all other mice. To further probe the differences, we generated a heatmap displaying the relative abundance of microbiome members at the family taxonomic level (FIG. 9K). Antibiotic-treated mice had a shift in microbiome composition that included increased levels of the species Bacteroides acidifaciens, Parabacteroides distasonis, Parabacteroides gordonii, and Lactobacillus ruminis, in addition to reduced levels of the species Mucispirillum schaedleri, Ruminococcus gnavus, and Desulfovibrio C21-c20 (FIG. 9L, FIG. 9M). These results supported the possibility of sublingual vaccines as a safe, long-term prophylactic approach to preventing urinary tract infections.

### Example 13

### Discussion

Detailed herein is the design of an anti-uropathogenic E. coli vaccine that leverages the unique advantages of a sublingual supramolecular nanofiber platform. A single highly co-assembled nanofiber containing a helper T-cell epitope raised simultaneous antibody responses against three UPEC peptide epitopes. Mice immunized with optimized formulations were protected from both systemic and transurethral UPEC challenge and induced minimal changes in the mouse microbiome.

This study presents a strategy with several advantages for progression towards clinical translation. Our vaccine strategy prioritizes safety, a critical consideration for a viable UTI vaccine. The threshold for safety is high for the design of a vaccine against a generally non-life threatening disease. Here, we showed the ability to raise systemic and mucosal responses against peptide UPEC epitopes that have previously been identified as ideal antigenic targets, but which suffered from poor immunogenicity. Two of these epitopes have previously been shown to raise no detectable responses even when adjuvanted with the strong but non-translatable full cholera toxin. In stark contrast, our vaccine raised not only systemic titers, but also epitope-specific antibody responses in urine while using the more favorable nucleotide adjuvants cyclic-di-AMP and CpG. Both classes of adjuvants, cyclic-di-nucleotides (such as AMP) and oligodeoxynucleotides (like CpG) have shown translational promise in clinical trials.

Due to the lack of an established UTI vaccine raising long-term responses in humans, some consequences of such a vaccine are not fully known. A non-specific vaccine is possible that, for example, raises responses that target both UPEC and non-pathogenic E. coli. Approaches that are based on the use of inactivated pathogens or lyophilized microbial proteins may contain epitopes that do not discriminate between pathogenic and non-pathogenic microbes. Given the importance of the microbiota and the damage that can result from continuous use of antibiotics, long-lasting off-target immune responses could have detrimental effects. We showed that nanofiber-generated antibodies bind to a UPEC strain and induce protection without showing any cross-reactivity towards a non-pathogenic E. coli strain. As an additional safeguard, our vaccine raised responses in the urinary tract and blood without eliciting detectable IgA antibodies in the feces, suggesting that there is minimal response in the commensal microbe-rich gastrointestinal tract. Most notably, our vaccine did not induce notable broad changes in the microbiome of mice. In stark contrast, mice treated with the oral antibiotic Fosfomycin had significantly altered microbiome composition. The importance of our vaccine's ability to raise protective responses without disrupting the microbiome is heightened by evidence suggesting microbiome changes can be passed down generationally.

The sublingual route not only affords the ability to raise systemic and urinary titers, but also provides key logistical benefits. Our tablet formulation has been shown to be heat-stable (Kelly, S. H., et al. Advanced healthcare materials 2021, 10, 2001614, incorporated herein by reference), which may reduce or eliminate the high costs and infrastructure required for cold-chain distribution of a vaccine and enable a far greater global distribution compared to vaccines that are dependent on refrigeration. The limitations of cold chain-dependent vaccine distribution have been acute and prominent during the SARS-CoV-2 pandemic, heightening awareness of this critical aspect of vaccine development. Additional advantages of sublingual tablet-based vaccination include the potential for self-administration, which could also help to facilitate its adoption. Our tablet vaccine raised responses not only in mice, but also in rabbits, which possess an oral cavity with key similarities to humans. Safety and efficacy studies in higher animal models may be done, but collectively, the results presented in this study provide a strong indicator of a favorable efficacy and safety profile.

One rationale for the use of the model UPEC strain CFT073 is that it was isolated from a human patient. This indicates that the epitope specificities of the antibodies we raised are clinically significant. How effective our vaccine is against other UPEC strains may be determined. Our multivalent strategy is designed so that most UPEC should express at least one of the three antigenic target proteins (IreA, lutA, IroN). For CFT073, our results suggest that expression of a single target may be sufficient. Expression varies significantly across UPEC, however, so testing our antibodies for binding against a panel of clinical isolates would give a greater indication of the predicted effects on a population level.

We focused on designing a vaccine against UPEC, which cause at least 80% of uncomplicated UTIs. The remaining 20% are caused by several genera of pathogens, including Staphylococcus, Klebsiella, Proteus, and Pseudomonas. Designing a “universal” UTI vaccine would present a challenge, particularly for a safety-focused approach such as this one that relies on peptide epitopes not shared with non-pathogenic microbes. However, the utilization of a supramolecular nanofiber-based vaccine does allow for the possibility of co-assembling a nanofiber containing a large number of epitopes.

### Example 14

### Coil29 and Q11 Systems Accommodate Multiple Peptides of Interest and Pro-Ala-Ser (PAS) Mucoinert Modifications

Different epitope peptides of interest bearing the Coil29 or Q11 assembly domains at their C-terminus were synthesized. Coil29 and Q11 variants with PAS modifications were also synthesized. All peptides were synthesized cleanly and were purified with reverse-phase HPLC. These peptides possessed the ability to be intermixed within their respective assembly systems (Q11 peptides together, or Coil29 peptides together, regardless of epitope) to form intermixed nanofibers bearing the epitopes of interest and PAS. The nanofibers were generally well-behaved, showing uniform thickness and lengths of hundreds of nanometers.

Prior work with sublingual immunizations was conducted with Q11 nanofibers, using PEGylation or PASylation to be sublingually immunogenic. However, the Coil29 system in principle possessed several potential advantages, including a non-amyloid structure, control over fiber length, and built-in T-cell epitopes. Mouse models of sublingual immunogenicity showed Coil29 nanofibers did not depend on PASylation for immunogenicity. In studies of mucoadhesion, Coil29—without PASylation—was highly non-adhesive to mucus. This may reflect the greater hydrophilicity of Coil29 nanofibers with respect to Q11 nanofibers. Both un-modified and OVA-bearing Coil29 nanofibers exhibited this muco-inertness, suggesting that elimination of PAS from this type of nanofiber may still retain good sublingual immunogenicity.

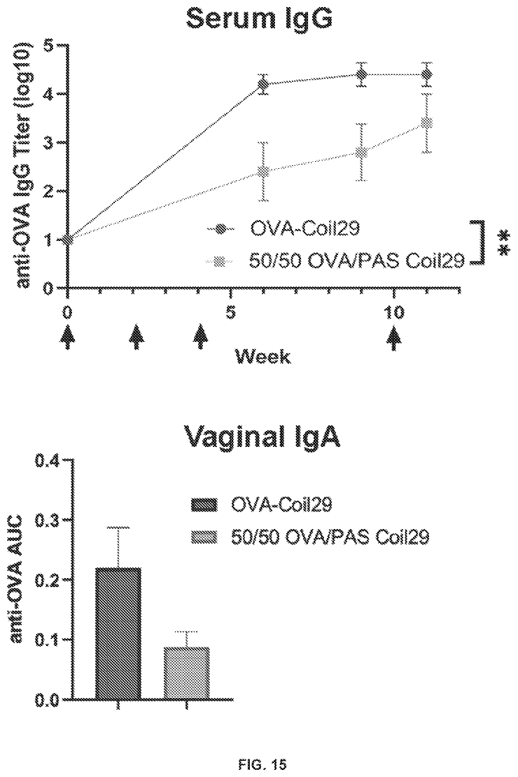

Further studies showed that OVA-Coil29 nanofibers were strongly immunogenic after three doses, raising both significant serum IgG titers (FIG. 15, top) and vaginal IgA titers (FIG. 15, bottom). Un-PASylated nanofibers displayed a higher IgG titer than those in which half of the peptides were PASylated (FIG. 15, top). Both formulations raised vaginal IgA, with a greater but not statistically different magnitude for un-PASylated nanofibers (FIG. 15, bottom).

The efficacy of sublingual immunization platforms for the Coil29 nanofiber system was epitope-dependent. Although Coil29 was immunogenic without any PASylation, a more hydrophobic epitope needed about 50% of the peptides in the nanofiber to be PASylated. Nanofibers with other epitopes were strongly immunogenic even in the absence of PASylation. In aggregate, the findings indicate that while Coil29 nanofibers present an attractive platform for sublingual vaccines against peptide epitopes, there is considerable variation in immunogenicity between various epitopes. PASylation can be utilized to overcome diminished immunogenicity. The physical properties of each epitope, namely hydrophobicity, may influence the mucoadhesivity of the nanofibers, with the most hydrophobic epitopes benefitting from PASylation, as PASylation diminished mucoadhesivity for each epitope studied, and it seems that mucoadhesion correlated with the hydrophobicity index of each peptide. The modularity of both the Q11 and Coil29 systems allows for straightforward adjustment of the degree of PASylation to achieve strong immunogenicity.

### Example 15

### Development of SIMPL Tablets for Coil29 Nanofibers

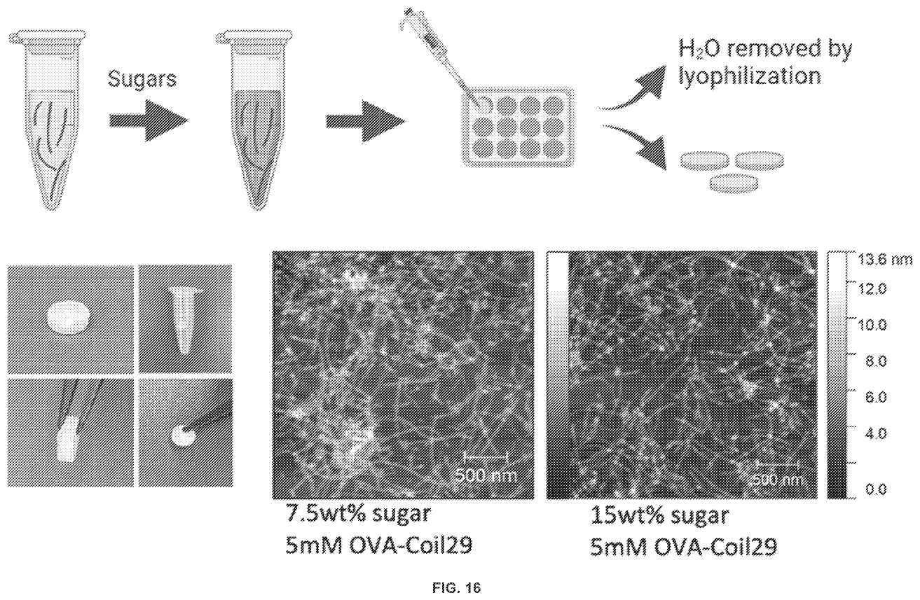

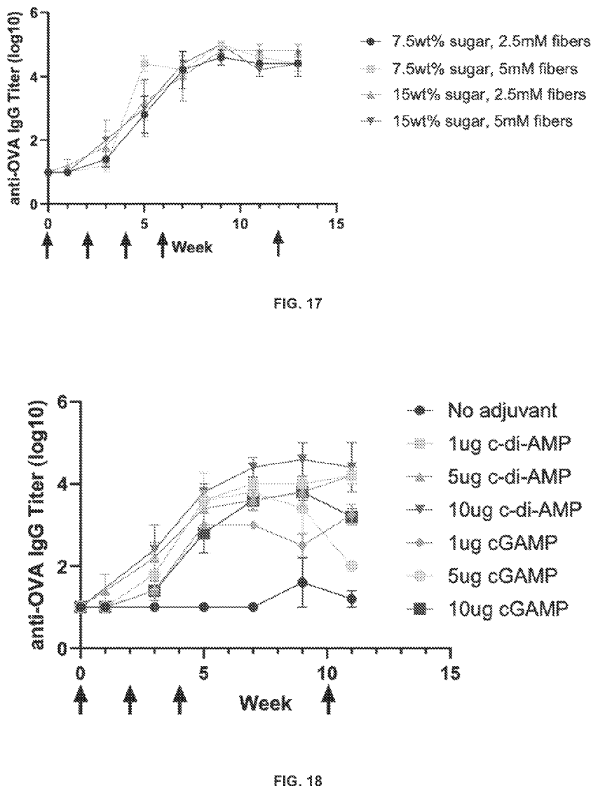

The tabletization process employed in the nanofiber-based sublingual immunotherapy tablets for the β-sheet nanofiber Q11 system was applied to make α-helical Coil29 nanofiber system tablets. The Coil29 system has advantages of being able to tune the length of the nanofibers, as well as having internal T-cell epitopes that may enhance antibody responses, however, it was examined whether the more dynamic helical nanofibers would be stable to the processing steps involved in freeze drying/tabletization and sublingual immunization. The tablets of the α-helical Coil29 nanofiber system showed that the Coil29 peptide assemblies retained their nanofibrillar structure in the presences of various levels of cryoprotectant sugars (FIG. 16). After tabletization, they also were sublingually immunogenic with a range of peptide concentration and cryoprotectant sugar concentrations (FIG. 17). Coil29 nanofibers also needed lower levels of STING adjuvants, c-di-AMP and cGAMP, as compared to Q11 nanofibers (FIG. 18). From these studies, it was observed that the adjuvant dose could be diminished to as low as 1 μg c-di-AMP with equivalent titers to 10 times that dose.

### ***

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A UPEC conjugate peptide comprising: (i) a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and (ii) at least one UPEC epitope conjugated to a terminus of the self-assembling peptide, wherein the UPEC epitope is selected from plroN (YLLYSKGNGCPKDITSGGCYLIGNKDLDPE, SEQ ID NO: 80), plutA (VDDIDYTQQQKIAAGKAISADAIPGGSVD, SEQ ID NO: 81), or plreA (GIAKAFRAPSIREVSPGFGTLTQGGASIMYGN, SEQ ID NO: 82), or a combination thereof.

Clause 2. The UPEC conjugate peptide of clause 1, wherein each self-assembling peptide forms a beta sheet and comprises a polypeptide having an amino acid sequence selected from QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn).

Clause 3. The UPEC conjugate peptide of clause 1, wherein each self-assembling peptide forms an alpha-helix and comprises a polypeptide having an amino acid sequence of bXXXb (SEQ ID NO: 1), wherein X is independently any amino acid and b is independently any positively charged amino acid.

Clause 4. The UPEC conjugate peptide according to clause 1 or 2, wherein the self-assembling peptide comprises the sequence QQKFQFQFEQQ (SEQ ID NO: 12) or Ac-QQKFQFQFEQQ-NH₂ (SEQ ID NO: 13) or bXXXb (SEQ ID NO: 1, wherein X is independently any amino acid and b is independently any positively charged amino acid).

Clause 5. The UPEC conjugate peptide of clause 1, 3, or 4, wherein b is independently selected from Arg and Lys.

Clause 6. The UPEC conjugate peptide of clause 1, 3, 4, or 5, wherein bXXXb (SEQ ID NO: 1) is RAYAR (SEQ ID NO: 2) or KAYAK (SEQ ID NO: 3).

Clause 7. The UPEC conjugate peptide of any one of clauses 1 and 3-6, wherein the self-assembling peptide comprises an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20.

Clause 8. The UPEC conjugate peptide of clause 7, wherein the self-assembling peptide comprises an amino acid sequence selected from

(Coil29, SEQ ID NO: 6)
QARILEADAEILRAYARILEAHAEILRAQ,
or
(SEQ ID NO: 7)
QAKILEADAEILKAYAKILEAHAEILKAQ,
or
(SEQ ID NO: 8)
ADAEILRAYARILEAHAEILRAQ,
or
(SEQ ID NO: 9)
Ac-QARILEADAEILRAYARILEAHAEILRAQ-NH₂,
or
(SEQ ID NO: 10)
Ac-QAKILEADAEILKAYAKILEAHAEILKAQ-NH₂,
or
(SEQ ID NO: 11)
Ac-ADAEILRAYARILEAHAEILRAQ-NH₂.

Clause 9. The UPEC conjugate peptide of any one of clauses 1-8, wherein the at least one UPEC epitope is attached to the C-terminus or the N-terminus of the self-assembling peptide.

Clause 10. The UPEC conjugate peptide of any one of clauses 1-9, wherein 1 to 10 UPEC epitopes are attached to the C-terminus or the N-terminus of the self-assembling peptide.

Clause 11. The UPEC conjugate peptide of any one of clauses 1-10, further comprising: (iii) a PEG molecule or a PAS peptide conjugated to the self-assembling peptide.

Clause 12. The UPEC conjugate peptide of clause 11, wherein the PAS peptide comprises a sequence of Pro-Ala-Ser or comprises the sequence of ASPAAPAPASPAAPAPSAPA (SEQ ID NO: 97), or a peptide having at least 80%, 85%, 90%, or 95% identity thereto.

Clause 13. The UPEC conjugate peptide of clause 11, wherein the PEG molecule comprises PEG-2000.

Clause 14. The UPEC conjugate of any one of clauses 11-13, wherein the PEG molecule or the PAS peptide is conjugated to the self-assembling peptide at the same or the opposite terminus from wherein the UPEC epitope is attached.

Clause 15. The UPEC conjugate peptide of any one of clauses 1-14, further comprising: (iv) at least one linker.

Clause 16. The UPEC conjugate peptide of clause 15, wherein the at least one linker comprises a first linker between the at least one UPEC epitope and the self-assembling peptide, and a second linker between the PEG molecule or the PAS peptide and the self-assembling peptide.

Clause 17. The UPEC conjugate peptide of clause 15 or 16, wherein the at least one linker comprises an amino acid sequence independently selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC)₈), SEQ ID NO: 92 ((G₄S)₃), SEQ ID NO: 93 (KSGSG), SEQ ID NO: 94 (KKSGSG), SEQ ID NO: 95 (EAAAK)₂, and SEQ ID NO: 96 (GGAAY).

Clause 18. The UPEC conjugate peptide of any one of clauses 1-17, wherein the UPEC conjugate peptide comprises a sequence selected from PEG-Q11-plroN, PEG-Q11-plutA, or PEG-Q11-plreA, or a combination thereof.

Clause 19. A nanofiber comprising a plurality of the UPEC conjugate peptide of any one of clauses 1-18, wherein the conjugate peptide self-assembles into the nanofiber.

Clause 20. A nanofiber comprising: (i) at least one UPEC conjugate peptide of any one of clauses 1-19; and (ii) at least one T-cell epitope-conjugate peptide comprising: a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and at least one T-cell epitope conjugated to a terminus of the self-assembling peptide, wherein the at least one T-cell epitope is selected from PADRE and VAC, and wherein PADRE comprises a polypeptide having the amino acid sequence of aKXVAAWTLKAa (SEQ ID NO: 99, wherein “X” comprises cyclohexylalanine and “a” comprises D-alanine), and wherein VAC comprises a polypeptide having the amino acid sequence of QLVFNSISARALKAY (SEQ ID NO: 100).

Clause 21. The nanofiber of clause 20, wherein the T-cell epitope-conjugate peptide further comprises a linker between the T-cell epitope and the self-assembling peptide.

Clause 22. The nanofiber of clause 21, wherein the linker comprises an amino acid sequence selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC)₈), SEQ ID NO: 92 ((G₄S)₃), SEQ ID NO: 93 (KSGSG), SEQ ID NO: 94 (KKSGSG), SEQ ID NO: 95 (EAAAK)₂, and SEQ ID NO: 96 (GGAAY).

Clause 23. The nanofiber of any one of clauses 20-22, wherein the cyclohexylalanine comprises D-alanine.

Clause 24. The nanofiber of any one of clauses 20-23, further comprising: (iii) a plain self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn).

Clause 25. The nanofiber of any one of clauses 20-24, wherein the nanofiber comprises a combination of 10 to 10,000, or 100 to 10,000 peptides comprising UPEC conjugate peptides and T-cell epitope-conjugate peptides.

Clause 26. The nanofiber of any one of clauses 20-24, wherein the nanofiber comprises a combination of 10 to 10,000, or 100 to 10,000 peptides comprising UPEC conjugate peptides, T-cell epitope-conjugate peptides, and plain self-assembling peptides.

Clause 27. The nanofiber of any one of clauses 20-26, wherein at least about 1%, 5%, 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, or 97.5% of the peptides in the nanofiber are UPEC conjugate peptides.

Clause 28. The nanofiber of any one of clauses 20-27, wherein at least about 1%, 2%, 3%, 4%, 5%, 6%, 7%, 8%, 9%, 10%, 11%, 12%, 13%, 14%, or 15% of the peptides in the nanofiber are T-cell epitope-conjugate peptides.

Clause 29. The nanofiber of any one of clauses 20-28, wherein the UPEC peptide conjugate and the T-cell epitope-peptide conjugate are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1.

Clause 30. The nanofiber of any one of clauses 20-29, wherein the self-assembling peptide forms a fibril including beta-sheet structures or a fibril having a coiled coil structure.

Clause 31. The nanofiber of any one of clauses 20-29, wherein the self-assembling peptide forms a fibril having a structure of a helical filament formed around a central axis.

Clause 32. The nanofiber of clause 31, wherein the N-terminus of each self-assembling peptide is positioned at the exterior of the helical filament.

Clause 33. The nanofiber of any one of clauses 20-32, wherein the UPEC molecules are exposed on the exterior surface of the nanofiber.

Clause 34. The nanofiber of any one of clauses 20-33, wherein the nanofiber is about 5-30 nm in width.

Clause 35. The nanofiber of any one of clauses 20-34, wherein the nanofiber is about 100 nm to 1 μm, 100 nm to 2 μm, 100 nm to 3 μm, 100 nm to 4 μm, or 100 nm to 5 μm in length.

Clause 36. A pharmaceutical composition comprising: (a) the UPEC conjugate peptide of any one of clauses 1-19 or the nanofiber of any one of clauses 20-35; and (b) a pharmaceutically acceptable carrier, diluent, and/or excipient.

Clause 37. The pharmaceutical composition of clause 36, further comprising: (c) an adjuvant selected from cyclic-di-AMP, CpG, cyclic GMP-AMP (cGAMP), cholera toxin B subunit (CTB), retinoic acid, or heat labile toxin B subunit, or a combination thereof.

Clause 38. The pharmaceutical composition of clause 36 or 37, formulated into a tablet.

Clause 39. The pharmaceutical composition of clause 38, wherein the tablet is a dissolving tablet.

Clause 40. A method of treating a urinary tract infection (UTI), the method comprising administering to a subject a therapeutically effective amount of the UPEC conjugate peptide of any one of clauses 1-19, or the nanofiber of any one of clauses 20-35, or the pharmaceutical composition of any one of clauses 36-39.

Clause 41. A method of treating a bacterial infection, the method comprising administering to a subject a therapeutically effective amount of the UPEC conjugate peptide of any one of clauses 1-19, or the nanofiber of any one of clauses 20-35, or the pharmaceutical composition of any one of clauses 36-39.

Clause 42. The method of clause 40 or 41, wherein the UPEC conjugate peptide or the nanofiber or the pharmaceutical composition is administered sublingually.

Clause 43. The method of any one of clauses 40-42, wherein pathogenic bacteria are reduced.

Clause 44. The method of clause 43, wherein pathogenic bacteria comprise uropathogenic Escherichia coli.

Clause 45. The method of clause 43 or 44, wherein pathogenic bacteria comprise CFT073.

Clause 46. The method of any one of clauses 40-45, wherein non-pathogenic bacteria are not reduced.

Clause 47. The method of any one of clauses 40-46, wherein the microbiome in the colon is maintained.

Clause 48. The method of any one of clauses 40-47, wherein the microbiome in the colon statistically maintains a Shannon Diversity Index.

SEQUENCES

SEQ ID NO: 1
bXXXb
wherein X is independently any amino acid, and b is independently any positively charged
amino acid.
SEQ ID NO: 2
RAYAR
SEQ ID NO: 3
KAYAK
SEQ ID NO: 4
RXXXR
wherein X is any amino acid.
SEQ ID NO: 5
ZₙbXXXbZₘ
wherein b is independently any positively charged amino acid, Z is independently any amino
acid, X is independently any amino acid, n is an integer from 0 to 20, and m is an integer
from 0 to 20.
SEQ ID NO: 6
Coil29
QARILEADAEILRAYARILEAHAEILRAQ
SEQ ID NO: 7
QAKILEADAEILKAYAKILEAHAEILKAQ
SEQ ID NO: 8
Coil23
ADAEILRAYARILEAHAEILRAQ
SEQ ID NO: 9
Ac-QARILEADAEILRAYARILEAHAEILRAQ-NH₂
SEQ ID NO: 10
Ac-QAKILEADAEILKAYAKILEAHAEILKAQ-NH₂
SEQ ID NO: 11
Ac-ADAEILRAYARILEAHAEILRAQ-NH₂
SEQ ID NO: 12
Q11
QQKFQFQFEQQ
SEQ ID NO: 13
AC-QQKFQFQFEQQ-NH2
FKFEFKFE (SEQ ID NO: 14)
KFQFQFE (SEQ ID NO: 15)
QQRFQFQFEQQ (SEQ ID NO: 16)
QQRFQWQFEQQ (SEQ ID NO: 17)
FEFEFKFKFEFEFKFK (SEQ ID NO: 18)
QQRFEWEFEQQ (SEQ ID NO: 19)
QQXFXWXFQQQ (SEQ ID NO: 20, where X is ornithine)
FKFEFKFEFKFE (SEQ ID NO: 21)
FKFQFKFQFKFQ (SEQ ID NO: 22)
AEAKAEAKAEAKAEAK (SEQ ID NO: 23)
AEAEAKAKAEAEAKAK (SEQ ID NO: 24)
AEAEAEAEAKAKAKAK (SEQ ID NO: 25)
RADARADARADARADA (SEQ ID NO: 26)
RARADADARARADADA (SEQ ID NO: 27)
SGRGYBLGGQGAGAAAAAGGAGQGGYGGLGSQG (SEQ ID NO: 28)
EWEXEXEXEX (SEQ ID NO: 29, where X is Val, Ala, Ser, or Pro)
WKXKXKXKXK (SEQ ID NO: 30 , where X is Val, Ala, Ser, or Pro)
KWKVKVKVKVKVKVK (SEQ ID NO: 31, where X is Val, A, Ser, or Pro)
LLLLKKKKKKKKLLLL (SEQ ID NO: 32)
VKVKVKVKVDPPTKVKVKVKV (SEQ ID NO: 33)
VKVKVKVKVDPPTKVKTKVKV (SEQ ID NO: 34)
KVKVKVKVKDPPSVKVKVKVK (SEQ ID NO: 35)
VKVKVKVKVDPPSKVKVKVKV (SEQ ID NO: 36)
VKVKVKTKVDPPTKVKTKVKV (SEQ ID NO: 37)
QQKFxFQFEQQ (SEQ ID NO: 38, wherein x is Glu, Asp, or Asn)
QQKFQxQFEQQ (SEQ ID NO: 39, wherein x is Trp or Tyr)
QQKFQFxFEQQ (SEQ ID NO: 40, wherein x is Glu, Asp, or Asn)
VEVKVEVKV (SEQ ID NO: 41)
VEVKVEVKVEVK (SEQ ID NO: 42)
VVVAAAEEE (SEQ ID NO: 43)
VEVEVEVEVEVEVEVEVEVE (SEQ ID NO: 44)
CGNKRTRGC (SEQ ID NO: 45)
VKVKVKVKVDPPTKVEVKVKV (SEQ ID NO: 46)
LRKKLGKA (SEQ ID NO: 47)
VVVVVVKK (SEQ ID NO: 48)
AEAKAEAKAEAKAEAK (SEQ ID NO: 49)
AEAKAEAK (SEQ ID NO: 50)
AEAEAEAEAKAK (SEQ ID NO: 51)
AEAEAKAK (SEQ ID NO: 52)
AEAEAKAKAEAEAKAK (SEQ ID NO: 53)
RADARADARADARADA (SEQ ID NO: 54)
RADARGDARADARGDA (SEQ ID NO: 55)
RADARADA (SEQ ID NO: 56)
RARADADARARADADA (SEQ ID NO: 57)
RARADADA (SEQ ID NO: 58)
RARARARADADADADA (SEQ ID NO: 59)
ADADADADARARARAR (SEQ ID NO: 60)
DADADADARARARARA (SEQ ID NO: 61)
RAEARAEARAEARAEA (SEQ ID NO: 62)
RAEARAEA (SEQ ID NO: 63)
KAKAKAKAEAEAEAEA (SEQ ID NO: 64)
AEAEAEAEAKAKAKAK (SEQ ID NO: 65)
KADAKADAKADAKADA (SEQ ID NO: 66)
KADAKADA (SEQ ID NO: 67)
AEAEAHAHAEAEAHAHA (SEQ ID NO: 68)
AEAEAHAHA (SEQ ID NO: 69)
HEHEHKHKHEHEHKHK (SEQ ID NO: 70)
HEHEHKHK (SEQ ID NO: 71)
FEFEFKFKFEFEFKFK (SEQ ID NO: 72)
FEFKFEFK (SEQ ID NO: 73)
LELELKLKLELELKLK (SEQ ID NO: 74)
LELELKLK (SEQ ID NO: 75)
KFDLKKDLKLDL (SEQ ID NO: 76)
FKFEFKFF (SEQ ID NO: 77)
FEFEFKFK (SEQ ID NO: 78)
RFRFRFRFRFRFRFRFRFRF (SEQ ID NO: 79)
SEQ ID NO: 80
UPEC epitope, plroN
YLLYSKGNGCPKDITSGGCYLIGNKDLDPE
SEQ ID NO: 81
UPEC epitope, plutA
VDDIDYTQQQKIAAGKAISADAIPGGSVD
SEQ ID NO: 82
UPEC epitope, plreA
GIAKAFRAPSIREVSPGFGTLTQGGASIMYGN
SEQ ID NO: 83
Linker
SGSG
SEQ ID NO: 84
Linker
(Ser-Gly)₂
SEQ ID NO: 85
Linker
CCCCSGSG
SEQ ID NO: 86
Linker
Gₙ wherein n is an integer from 1 to 10
SEQ ID NO: 87
Linker
GSGS
SEQ ID NO: 88
Linker
SSSS
SEQ ID NO: 89
Linker
GGGS
SEQ ID NO: 90
Linker
GGC
SEQ ID NO: 91
Linker
(GGC)₈
SEQ ID NO: 92
Linker
(G4S)₃
SEQ ID NO: 93
Linker
KSGSG
SEQ ID NO: 94
Linker
KKSGSG
SEQ ID NO: 95
Linker
(EAAAK)₂
SEQ ID NO: 96
Linker
GGAAY
SEQ ID NO: 97
PAS peptide
ASPAAPAPASPAAPAPSAPA
SEQ ID NO: 98
PAS peptide
H₂N-ASPAAPAPASPAAPAPSAPA-NH₂
SEQ ID NO; 99
PADRE molecule
aKXVAAWTLKAa, wherein “X” comprises cyclohexylalanine and “a” comprises D-alanine
SEQ ID NO: 100
VAC molecule
QLVFNSISARALKAY
SEQ ID NO: 101
mPEG₂₀₀₀-plroN-Q11
mPEG₂₀₀₀-SGSG-QQKFQFQFEQQ-SGSG-YLLYSKGNGCPKDITSGGCYLIGNKDLDPE-
NH₂
SEQ ID NO: 102
mPEG₂₀₀₀-plutA-Q11
mPEG₂₀₀₀-SGSG-QQKFQFQFEQQ-SGSG-VDDIDYTQQQKIAAGKAISADAIPGGSVD-
NH₂
SEQ ID NO: 103
mPEG₂₀₀₀-plreA-Q11
mPEG₂₀₀₀-SGSG-QQKFQFQFEQQ-SGSG-
GIAKAFRAPSIREVSPGFGTLTQGGASIMYGN-NH₂
SEQ ID NO: 104
PADRE-Q11
H₂N-aKXVAAWTLKAa-SGSG-QQKFQFQFEQQ-NH₂
SEQ ID NO: 105
VAC-Q11
H₂N-QLVFNSISARALKAY-SGSG-QQKFQFQFEQQ-NH₂
SEQ ID NO: 106
PADRE-Q11
aKXVAAWTLKAa-SGSG-QQKFQFQFEQQ
SEQ ID NO: 107
VAC-Q11
QLVFNSISARALKAY-SGSG-QQKFQFQFEQQ

What is claimed is:

1. A UPEC conjugate peptide comprising:
(i) a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16, QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33, VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and
(ii) at least one UPEC epitope conjugated to a terminus of the self-assembling peptide, wherein the UPEC epitope is selected from plroN (YLLYSKGNGCPKDITSGGCYLIGNKDLDPE, SEQ ID NO: 80), plutA (VDDIDYTQQQKIAAGKAISADAIPGGSVD, SEQ ID NO: 81), or plreA (GIAKAFRAPSIREVSPGFGTLTQGGASIMYGN, SEQ ID NO: 82), or a combination thereof.

2. The UPEC conjugate peptide of claim 1, wherein each self-assembling peptide forms a beta sheet and comprises a polypeptide having an amino acid sequence selected from QQKFQFQFEQQ (SEQ ID NO: 12), FKFEFKFE (SEQ ID NO: 14), KFQFQFE (SEQ ID NO: 15, QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21, FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33, VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn).

3. The UPEC conjugate peptide of claim 1, wherein each self-assembling peptide forms an alpha-helix and comprises a polypeptide having an amino acid sequence of bXXXb (SEQ ID NO: 1), wherein X is independently any amino acid and b is independently any positively charged amino acid.

4. The UPEC conjugate peptide according to claim 1 or 2, wherein the self-assembling peptide comprises the sequence QQKFQFQFEQQ (SEQ ID NO: 12) or Ac-QQKFQFQFEQQ-NH₂ (SEQ ID NO: 13) or bXXXb (SEQ ID NO: 1, wherein X is independently any amino acid and b is independently any positively charged amino acid).

5. The UPEC conjugate peptide of claim 1, 3, or 4, wherein b is independently selected from Arg and Lys.

6. The UPEC conjugate peptide of claim 1, 3, 4, or 5, wherein bXXXb (SEQ ID NO: 1) is RAYAR (SEQ ID NO: 2) or KAYAK (SEQ ID NO: 3).

7. The UPEC conjugate peptide of any one of claims 1 and 3-6, wherein the self-assembling peptide comprises an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20.

8. The UPEC conjugate peptide of claim 7, wherein the self-assembling peptide comprises an amino acid sequence selected from QARILEADAEILRAYARILEAHAEILRAQ (Coil29, SEQ ID NO: 6), or QAKILEADAEILKAYAKILEAHAEILKAQ (SEQ ID NO: 7), or ADAEILRAYARILEAHAEILRAQ (SEQ ID NO: 8), or Ac-QARILEADAEILRAYARILEAHAEILRAQ-NH₂ (SEQ ID NO: 9), or Ac-QAKILEADAEILKAYAKILEAHAEILKAQ-NH₂ (SEQ ID NO: 10), or Ac-ADAEILRAYARILEAHAEILRAQ-NH₂ (SEQ ID NO: 11).

9. The UPEC conjugate peptide of any one of claims 1-8, wherein the at least one UPEC epitope is attached to the C-terminus or the N-terminus of the self-assembling peptide.

10. The UPEC conjugate peptide of any one of claims 1-9, wherein 1 to 10 UPEC epitopes are attached to the C-terminus or the N-terminus of the self-assembling peptide.

11. The UPEC conjugate peptide of any one of claims 1-10, further comprising:
(iii) a PEG molecule or a PAS peptide conjugated to the self-assembling peptide.

12. The UPEC conjugate peptide of claim 11, wherein the PAS peptide comprises a sequence of Pro-Ala-Ser or comprises the sequence of ASPAAPAPASPAAPAPSAPA (SEQ ID NO: 97), or a peptide having at least 80%, 85%, 90%, or 95% identity thereto.

13. The UPEC conjugate peptide of claim 11, wherein the PEG molecule comprises PEG-2000.

14. The UPEC conjugate of any one of claims 11-13, wherein the PEG molecule or the PAS peptide is conjugated to the self-assembling peptide at the same or the opposite terminus from wherein the UPEC epitope is attached.

15. The UPEC conjugate peptide of any one of claims 1-14, further comprising:
(iv) at least one linker.

16. The UPEC conjugate peptide of claim 15, wherein the at least one linker comprises a first linker between the at least one UPEC epitope and the self-assembling peptide, and a second linker between the PEG molecule or the PAS peptide and the self-assembling peptide.

17. The UPEC conjugate peptide of claim 15 or 16, wherein the at least one linker comprises an amino acid sequence independently selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC)₈), SEQ ID NO: 92 ((G₄S)₃), SEQ ID NO: 93 (KSGSG), SEQ ID NO: 94 (KKSGSG), SEQ ID NO: 95 (EAAAK)₂, and SEQ ID NO: 96 (GGAAY).

18. The UPEC conjugate peptide of any one of claims 1-17, wherein the UPEC conjugate peptide comprises a sequence selected from PEG-Q11-plroN, PEG-Q11-plutA, or PEG-Q11-plreA, or a combination thereof.

19. A nanofiber comprising a plurality of the UPEC conjugate peptide of any one of claims 1-18, wherein the conjugate peptide self-assembles into the nanofiber.

20. A nanofiber comprising:
(i) at least one UPEC conjugate peptide of any one of claims 1-19; and
(ii) at least one T-cell epitope-conjugate peptide comprising:
a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and
at least one T-cell epitope conjugated to a terminus of the self-assembling peptide, wherein the at least one T-cell epitope is selected from PADRE and VAC, and wherein PADRE comprises a polypeptide having the amino acid sequence of aKXVAAWTLKAa (SEQ ID NO: 99, wherein “X” comprises cyclohexylalanine and “a” comprises D-alanine), and wherein VAC comprises a polypeptide having the amino acid sequence of QLVFNSISARALKAY (SEQ ID NO: 100).

21. The nanofiber of claim 20, wherein the T-cell epitope-conjugate peptide further comprises a linker between the T-cell epitope and the self-assembling peptide.

22. The nanofiber of claim 21, wherein the linker comprises an amino acid sequence selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC)₈), SEQ ID NO: 92 ((G₄S)₃), SEQ ID NO: 93 (KSGSG), SEQ ID NO: 94 (KKSGSG), SEQ ID NO: 95 (EAAAK)₂, and SEQ ID NO: 96 (GGAAY).

23. The nanofiber of any one of claims 20-22, wherein the cyclohexylalanine comprises D-alanine.

24. The nanofiber of any one of claims 20-23, further comprising:
(iii) a plain self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16, QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33, VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn).

25. The nanofiber of any one of claims 20-24, wherein the nanofiber comprises a combination of 10 to 10,000, or 100 to 10,000 peptides comprising UPEC conjugate peptides and T-cell epitope-conjugate peptides.

26. The nanofiber of any one of claims 20-24, wherein the nanofiber comprises a combination of 10 to 10,000, or 100 to 10,000 peptides comprising UPEC conjugate peptides, T-cell epitope-conjugate peptides, and plain self-assembling peptides.

27. The nanofiber of any one of claims 20-26, wherein at least about 1%, 5%, 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, or 97.5% of the peptides in the nanofiber are UPEC conjugate peptides.

28. The nanofiber of any one of claims 20-27, wherein at least about 1%, 2%, 3%, 4%, 5%, 6%, 7%, 8%, 9%, 10%, 11%, 12%, 13%, 14%, or 15% of the peptides in the nanofiber are T-cell epitope-conjugate peptides.

29. The nanofiber of any one of claims 20-28, wherein the UPEC peptide conjugate and the T-cell epitope-peptide conjugate are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1.

30. The nanofiber of any one of claims 20-29, wherein the self-assembling peptide forms a fibril including beta-sheet structures or a fibril having a coiled coil structure.

31. The nanofiber of any one of claims 20-29, wherein the self-assembling peptide forms a fibril having a structure of a helical filament formed around a central axis.

32. The nanofiber of claim 31, wherein the N-terminus of each self-assembling peptide is positioned at the exterior of the helical filament.

33. The nanofiber of any one of claims 20-32, wherein the UPEC molecules are exposed on the exterior surface of the nanofiber.

34. The nanofiber of any one of claims 20-33, wherein the nanofiber is about 5-30 nm in width.

35. The nanofiber of any one of claims 20-34, wherein the nanofiber is about 100 nm to 1 μm, 100 nm to 2 μm, 100 nm to 3 μm, 100 nm to 4 μm, or 100 nm to 5 μm in length.

36. A pharmaceutical composition comprising: (a) the UPEC conjugate peptide of any one of claims 1-19 or the nanofiber of any one of claims 20-35; and (b) a pharmaceutically acceptable carrier, diluent, and/or excipient.

37. The pharmaceutical composition of claim 36, further comprising: (c) an adjuvant selected from cyclic-di-AMP, CpG, cyclic GMP-AMP (cGAMP), cholera toxin B subunit (CTB), retinoic acid, or heat labile toxin B subunit, or a combination thereof.

38. The pharmaceutical composition of claim 36 or 37, formulated into a tablet.

39. The pharmaceutical composition of claim 38, wherein the tablet is a dissolving tablet.

40. A method of treating a urinary tract infection (UTI), the method comprising administering to a subject a therapeutically effective amount of the UPEC conjugate peptide of any one of claims 1-19, or the nanofiber of any one of claims 20-35, or the pharmaceutical composition of any one of claims 36-39.

41. A method of treating a bacterial infection, the method comprising administering to a subject a therapeutically effective amount of the UPEC conjugate peptide of any one of claims 1-19, or the nanofiber of any one of claims 20-35, or the pharmaceutical composition of any one of claims 36-39.

42. The method of claim 40 or 41, wherein the UPEC conjugate peptide or the nanofiber or the pharmaceutical composition is administered sublingually.

43. The method of any one of claims 40-42, wherein pathogenic bacteria are reduced.

44. The method of claim 43, wherein pathogenic bacteria comprise uropathogenic Escherichia coli.

45. The method of claim 43 or 44, wherein pathogenic bacteria comprise CFT073.

46. The method of any one of claims 40-45, wherein non-pathogenic bacteria are not reduced.

47. The method of any one of claims 40-46, wherein the microbiome in the colon is maintained.

48. The method of any one of claims 40-47, wherein the microbiome in the colon statistically maintains a Shannon Diversity Index.
